(19) United States
(12) Patent Application Publication
(10) Pub. No.: US 2013/0022590 A1
(43) Pub. Date: Jan. 24, 2013

(54) Compositions Comprising Zinc Finger Domains and Uses Therefor

(71) Applicants: Joel MacKay, Newtown (AU); Mitchell O'Connell, Newton (AU)

(72) Inventors: Joel MacKay, Newtown (AU); Mitchell O'Connell, Newton (AU)

(21) Appl. No.: 13/514,946

(22) PCT Filed: Apr. 6, 2010
(86) PCT No.: PCT/AU10/00392

§ 371 Date: Sep. 11, 2012

Related U.S. Application Data
(60) Provisional application No. 61/285,924, filed on Dec. 11, 2009.

(51) Int. Cl. C07K 14/435; C12N 15/63; C12N 15/82; A61K 38/46; C12N 5/071; C12N 15/85; C12N 15/12; A61K 38/17

(52) U.S. Cl. 424/94.6; 530/350; 536/23.5; 435/320.1; 514/21.2; 435/375

(57) ABSTRACT

The present invention relates to compositions of matter comprising zinc finger domains that bind to single-stranded RNA and are useful for modifying gene expression such as by regulating processing of messenger RNA (mRNA) or non-coding RNA (ncRNA). The invention also relates to screening, diagnostic and therapeutic methods employing such compositions of matter.

### CROSS-REFERENCE TO RELATED APPLICATIONS

This application claims the benefit of priority from U.S. Application No. 61/285,924 filed Dec. 11, 2009-the-entire content of which is incorporated herein.

### FIELD OF THE INVENTION

The present invention relates to peptide-based compositions, analogs thereof and their use in agriculture, veterinary applications and medicine, for example in a method of diagnosis and/or prognosis and/or therapy of the human or animal body or in an ex vivo method of diagnosis and/or prognosis and/or therapy of the human or animal body. More particularly, the present invention relates to compositions of matter comprising zinc finger domains that bind to single-stranded RNA and are useful for modifying gene expression such as by regulating processing of messenger RNA (mRNA) or non-coding RNA (ncRNA). The invention also relates to screening, diagnostic and therapeutic methods employing such compositions of matter.

### BACKGROUND OF THE INVENTION

### 1. Description of the Related Art

In complex organisms, phenotypic diversity is achieved primarily by the regulation of gene expression. Messenger RNA not only provides a template for translation, but can be used both to control the levels of the encoded protein (by regulating the transport, translation, storage and degradation of the message) and to modulate the function of that protein through alternative splicing.

a) RNA Processing and Disease

Approximately 15% of all diseases arise from aberrant or inappropriate RNA processing. For example, specific mutations in the dystrophin gene cause Duchenne muscular dystrophy (DMD) by eliminating a binding site in the mRNA for a splicing factor and thereby inducing exon skipping or premature termination (Shiga et al., J. Clin. Invest. 100: 2204-10, 1997). Similarly, nonsense mutations in BRCA1 (Mazyoer et al., Am. J. Hum Genet. 62:713-5, 1998) and fibrillin 1 (Dietz et al., Science, 259: 680-3, 1993) also cause inappropriate exon skipping and are associated with breast cancer and Marfan syndrome, respectively. RNA is also intimately involved in a range of other disorders. For example, all retroviral infections require the injection of the retroviral single stranded RNA genome into the cytoplasm of the host cell.

Notwithstanding that many genes are alternatively spliced, and improper splicing forms the basis for many diseases, our understanding of the mechanisms for RNA-binding proteins in controlling mRNA splicing is rudimentary.

b) Single-Stranded RNA (ssRNA)

Single-stranded RNA (ssRNA) has been implicated in regulating gene expression in prokaryotes and eukaryotes, and forms the basis of many viral genomes e.g., positive-sense viruses, negative-sense viruses, and ambisense viruses.

Exemplary RNA viruses include those belonging to Group III, Group IV or Group V of the Baltimore classification of viruses i.e., excluding retroviruses that form a DNA intermediate during viral replication, however other classification systems RNA viruses are termed riboviruses i.e., including retroviruses. In another classification system, Exemplary diseases caused by RNA viruses include SARS, influenza and hepatitis C, and a large proportion of plant viruses are RNA viruses. In view of the importance of RNA viruses and riboviruses to human and animal health and to crop yield, the efficient regulation of ssRNA processing is important for controlling virus transmission in medical, veterinary and agricultural contexts.

Regulatory ssRNA comprises any single-stranded RNA that is functional to modify gene expression, including non-coding RNA (ncRNA). In common with mRNA having single-stranded regions that function in regulation of gene expression, a considerable fraction of ncRNAs appear to be single stranded RNA (ssRNA).

Micro RNA (miRNA) represses mRNA translation and/or hasten their degradation by binding to complementary sequences in the 3′-UTR of target genes, thereby recruiting Argonaute proteins e.g., TP2. For example, in higher eukaryotes miRNAs are known to regulate gene expression by forming duplexes with complementary sequences in mRNA, generally in the 3′ UTR thereby down-regulating expression. In one example, a metazoan ncRNA known as 7SK RNA acts as a negative regulator of the RNA polymerase II elongation factor P-TEFb in response to stress. In another example, bacterial 6S RNA is known to associate with the RNA polymerase holoenzyme containing the sigma70 specificity factor to repress expression from a sigma70-dependent promoter during stationary phase. In yet another example, Escherichia coli OxyS RNA is known to repress translation in response to oxidative stress, by binding to Shine-Dalgarno sequences, thereby occluding ribosome binding. In yet another example, the B2 RNA transcripts are known to repress mRNA transcription by binding to core Pol II in response to heat shock in mouse cells, thereby assembling into a preinitiation complex at the promoter and blocking RNA synthesis. In yet another example, it is known that RNA polymerase II transcription of ncRNAs is required for chromatin remodelling in the yeast Schizosaccharomyces pombe, wherein the chromatin is progressively converted to an open configuration as several species of ncRNAs are transcribed.

Dysregulation of miRNA is known to be associated with diseases in plants and animals, including humans. A manually-curated database incorporated herein by reference documents known relationships between miRNA dysregulation and human disease e.g., Jiang et al., Nuc. Acids Res. 37, D98-104 (2009). Several miRNAs have links with some types of cancer. For example, in a murine model of cancer development, mice treated with lymphoma cells over-expressing miRNA developed disease within 50 days and died two weeks later, compared to negative control animals that lived for more than 100 days e.g., He et al., Nature 435, 828-33 (2005). Leukemia have also been shown to be produced by over-expression of miRNA e.g., Cui et al., Blood 110, 2631-2640 (2007). In another example, two types of miRNA are known to inhibit the E2F1 protein, to thereby regulate cell proliferation e.g., O'Donnell et al., Nature 435, 839-943 (2005). In another example, variations in the miRNAs miR-17 and miR-30c-1 are associated with regulation of breast cancer-associated genes in patients that are non-carriers of BRCA1 or BRCA2 mutations, suggesting that familial breast cancer may be caused by variation in these miRNAs e.g., Shen et al., Int J Cancer 124, 1178-1182 (2009).

It is also apparent that miRNAs play a role in cardiac development and function. For example, expression levels of specific miRNAs are modified in diseased human hearts, suggesting their involvement in cardiomyopathy e.g., Thum et al., Circulation 116, 258-267 (2007); van Rooij et al., Proc. Natl. Acad. Sci. U.S.A. 103, 18255-18260 (2006); Tatsuguchi et al., J. Mol. Cell. Cardiol. 42, 1137-1141 (2006). In other examples, studies in animal models have identified distinct roles for specific miRNAs during heart development and under pathological conditions, including the regulation of key factors important for cardiogenesis, the hypertrophic growth response, and cardiac conductance e.g., Zhao et al., Nature 436, 214-220 (2005); Xiao et al., J. Biol. Chem. 282, 12363-12367 (2007); Yang et al., Nat. Med. 13, 486-491 (2007); Care et al., Nat. Med. 13, 613-618 (2007); van Rooij et al., Science 316, 575-579 (2007).

As used herein, the term “non-coding RNA” or “ncRNA” includes a functional RNA molecule that is not translated into a protein e.g., non-protein-coding RNA (npcRNA), non-messenger RNA (mRNA), small non-messenger RNA (smRNA), functional RNA (fRNA), or small RNA (sRNA). exemplary ncRNAs include highly abundant and functionally important RNAs such as transfer RNA (tRNA) and/or ribosomal RNA (rRNA) and/or snoRNA and/or microRNA (miRNA) and/or small inhibitory RNA (siRNA) and/or Piwi-interacting RNA (piRNA) and/or long ncRNAs e.g., Xist or HOTAIR.

The number of ncRNAs encoded within the human genome is unknown, however recent transcriptomic and bioinformatic studies suggest the existence of thousands of ncRNAs.

A number of ncRNAs are known to be embedded in the 5′ UTRs of protein-coding genes to influence gene expression. A “riboswitch” can directly bind a small target molecule, to modify gene expression. For example, RNA leader sequences (e.g., a histidine operon leader, a leucine operon leader, a threonine operon leader or a tryptophan operon leader) are known to occur upstream of the first gene of amino acid biosynthetic operons, which form one of two possible structures in regions encoding very short peptide sequences that are rich in the end product amino acid of the operon, wherein a terminator structure forms when there is an excess of a regulatory amino acid and ribosome movement over the leader transcript is unimpeded, however a deficiency of the charged tRNA of a regulatory amino acid causes the ribosome translating the leader peptide to stall and form an anti-terminator structure allowing RNA polymerase to transcribe the operon. In other examples, cis-acting response elements that bind trans-acting regulatory proteins are known to occur in the 5′-untranslated and/or 3′-untranslated regions of many prokaryotic and eukaryotic mRNAs that regulate gene expression at the post-transcriptional level e.g., by modifying translation in response to RNA-protein interactions, either by up-regulating or down-regulating gene expression. In yet other examples, internal ribosome entry sites (IRES) are known to be RNA structures that facilitate translational initiation e.g., at an internal site in mRNA.

Piwi-interacting RNAs (piRNAs) expressed in mammalian testes and somatic cells form RNA-protein complexes with Piwi proteins i.e., piRNA complexes (piRCs), that are implicated in transcriptional gene silencing of retrotransposons in germ line cells, e.g., during spermatogenesis.

Xist (X-inactive-specific transcript) is a long ncRNA gene on the X chromosome in placental mammals that acts as major effector of the X chromosome inactivation process forming Barr bodies. An antisense RNA (Tsix) is a negative regulator of Xist such that cells lacking Tsix expression and having high levels of Xist transcription are undergo more frequent X inactivation than otherwise. In drosophilids, the roX (RNA on the X) RNA is known to be involved in dosage compensation, wherein Xist and roX operate by epigenetic regulation of transcription through the recruitment of histone-modifying enzymes.

Mutations or imbalances in the ncRNA repertoire within the body can cause a variety of diseases. For example, ncRNAs may have abnormal expression patterns in cancerous tissues e.g., long mRNA-like ncRNAs e.g., Pibouin et al. Cancer Genet Cytogenet 133, 55-60 (2002); Fu et al., DNA Cell Biol. 25, 135-141 (2006). In another example, germ-line mutations in miR-16-1 and miR-15 primary precursors are more abundant in patients with chronic lymphocytic leukemia compared to control populations e.g., Calin et al., N Engl. J. Med. 353, 1793-1801 (2005); Cain et al., Proc Natl Acad Sci USA 99, 15524-15529 (2002).

In yet another example, a deletion of 48 copies of the C/D box snoRNA (SNORD116) has been shown to be the primary cause of Prader-Willi syndrome, a developmental disorder associated with over-eating and learning difficulties. SNORD116 has potential target sites within a number of protein-coding genes, and could have a role in regulating alternative splicing e.g., Bazeley et al., Gene 408, 172-179 (2008).

In another example, the chromosomal locus containing the small nucleolar RNA SNORD115 gene cluster is duplicated in approximately 5% of individuals with autistic traits, and a mouse model of autism comprises a duplication of the SNORD115 cluster e.g., Nakatani et al., Cell 137, 1235-1246 (2009).

In yet another example of the role of ssRNA in disease, antisense RNA (BACE1-AS) is known to be up-regulated in patients suffering from Alzheimer's disease and in amyloid precursor protein transgenic mice e.g., Faghihi et al., Nat Med 14, 723-730 (2008).

In view of the extensive role of ssRNA in regulating gene expression in prokaryotes and eukaryotes, and the associations of ssRNA with infection and disease in humans and other animals, and in plants, it is clearly desirable to develop means for regulating gene expression at the level of ssRNA.

c) Zinc Finger Proteins and Regulation of Gene Expression

Zinc finger proteins are known to be DNA-binding proteins comprising two or three zinc finger domains, that form transcription factors conferring DNA sequence specificity as the DNA-binding domain.

Classical zinc-finger (ZnF) proteins are known in the art to comprise small independently folded domains of ˜30-amino-acid comprising conserved positioning/spacing of cysteine residues and histidines, such as various Cys-Cys (C-C) or Cys-His (C-H) motifs, coordinated to zinc. Exemplary zinc finger domains recognized in the art include the C₂⁻H₂ zinc finger, Gag knuckle, treble clef finger, zinc ribbon, Zn2/Cys6-like finger, TAZ2 domain-like finger, short zinc-binding loop, and metallothionein fold see e.g., Sri Krishna et al., Nucleic Acids Res. 31, 532-550 (2003) incorporated by reference herein. For example, C₂⁻H₂ zinc finger proteins are known to comprise possibly the largest family of regulatory proteins in mammals, most of which bind DNA and/or RNA, wherein the binding properties depend on the amino acid sequence of the finger domains and of the linker region between fingers, as well as on the higher-order structures and the number of fingers e.g., see Iuchi, Cell. Mol. Life. Sci. 58, 628-635 (2001). The art also recognized that C₂H₂ zinc finger proteins may contain from 1 to more than 30 fingers and, may be classified into three groups based on the number and the pattern of the fingers viz., triple-C₂H₂ that generally bind to a single legend type, multiple-adjacent-C₂H₂ that can bind multiple and different ligands, and separated-paired-C₂H₂ finger proteins which generally bind to the target nucleic acid by means of only a single pair.

In one specific example, the ZnF protein ZRANB2 (syn. Z is, ZNF265) is an SR-like nuclear protein that displays 2 N-terminal zinc fingers (ZnFs), and is expressed in most tissues and is conserved between nematodes and mammals. It interacts with the spliceosomal proteins U1-70K and U2AF³⁵ and can alter the distribution of splice variants of GluR-B, SMN2, and Tra2β in minigene reporter assays. The zinc finger domain of ZRANB2 comprises 2 distorted β-hairpins sandwiching a central tryptophan (W) residue and a single zinc ion e.g., Plambeck et al., J. Biol. Chem. 278, 22805-22811 (2003); Wang et al., J. Biol. Chem. 278, 20225-20234 (2003).

Each zinc finger domain binds dsDNA by inserting the α-helix into the DNA major groove thereby utilizing electrostatic and hydrophobic interactions to bind three base pairs of DNA. Due to the modularity of the zinc finger domain(s), ZnF proteins have utility in protein engineering.

ZnF proteins have been reported to have a role in mRNA processing, however until the present invention the structural basis for the ZnF-RNA interaction was believed to be mediated via double-stranded regions in the RNA target e.g., stem regions of hairpin loops, see e.g., De Guzman et al., Science, 279:384-8.8 (1998). Binding of ZnF proteins to ssRNA and the structural basis for any such interaction, is not known or well-explored.

### 2. General Information

The following prior disclosures provide conventional techniques of molecular biology, microbiology, virology, recombinant DNA technology, peptide synthesis in solution, solid phase peptide synthesis, and immunology:
1. Sambrook, Fritsch & Maniatis, Molecular Cloning: A Laboratory Manual, Cold Spring Harbor Laboratories, New York, Third Edition (2001), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, ppl-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B., A Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactiva, Germany);
10. Sakakibara, D., Teichman, J., Lien, E. Land Fenichel, R. L. (1976). Biochem. Biophys. Res. Commun. 73 336-342
11. Merrifield, R. B. (1963). J. Am. Chem. Soc. 85, 2149-2154.
12. Barany, G. and Merrifield, R. B. (1979) in The Peptides (Gross, E. and Meienhofer, J. eds.), vol. 2, pp. 1-284, Academic Press, New York.
13. Wünsch, E., ed. (1974) Synthese von Peptiden in Houben-Weyls Metoden der Organischen Chemie (Müller, E., ed.), vol. 15, 4th edn., Parts 1 and 2, Thieme, Stuttgart.
14. Bodanszky, M. (1984) Principles of Peptide Synthesis, Springer-Verlag, Heidelberg.
15. Bodanszky, M. & Bodanszky, A. (1984) The Practice of Peptide Synthesis, Springer-Verlag, Heidelberg.
16. Bodanszky, M. (1985) Int. J. Peptide Protein Res. 25, 449-474.
17. McPherson et al., In: PCR A Practical Approach., IRL Press, Oxford University Press, Oxford, United Kingdom, 1991.
18. Methods in Yeast Genetics: A Cold Spring Harbor Laboratory Course Manual (D. Burke et al., eds) Cold Spring Harbor Press, New York, 2000 (see whole of text).
19. Guide to Yeast Genetics and Molecular Biology. In: Methods in Enzymology Series, Vol. 194 (C. Guthrie and G. R. Fink eds) Academic Press, London, 1991 2000 (see whole of text).

### SUMMARY OF INVENTION

In work leading up to the present invention, the inventors sought to determine whether or not ZnF proteins bind to ssRNA, using a RanBP2 zinc finger domain structure comprising Structural Formula I:

W-X-C-X₂₋₄-C-X₃-N-X₆-C-X₂-C.

As ZRANB2 appears to regulate splice site choice, the inventors reasoned that RanBP2-type zinc finger domains that do actually bind ssRNA would a model system for investigating and/or regulating cellular processes and for designing RNA-based therapeutics.

As used herein and unless the context requires otherwise, the term “RanBP2-type” shall be taken to refer to a zinc finger domain comprising Structural Formula II:

X₂₋₃-Za-X₀₋₁-W-X-C-X₂₋₄-C-X-Zb-X₂-Zc-X-Zd-Ze-X₂-C-Zf-X-C

wherein each of X, Za, Zb, Zc, Zd, Ze and Zf is an amino acid, and wherein a side chain of any one or more of Za to Zf is functional to contact at least one residue of single-stranded RNA.

The inventors also sought to determine the structural basis for interaction between RanBP2-type zinc finger domain(s) and target ssRNA(s), by defining a sub-genus of ssRNA-binding RanBP2-type zinc finger domains, optimized target recognition sequence(s) in ssRNA, and optimized spacing between RanBP2-type zinc finger domains in polypeptide scaffolds. The inventors have thus provided the means for generating compositions of matter for regulating expression of a class of ssRNAs, including combinatorial libraries of scaffolds expressing a plurality of zinc finger domains e.g., linked in cis, and pharmaceutical compositions for use in medicine. The inventors have also provided the means for modifying gene expression e.g., the inhibition or reduction of viral gene expression, such as in the treatment or prevention of diseases in plants, animals and humans.

More particularly, the data exemplified herein demonstrates that a class of RanBP2-type zinc finger domains, including that from ZRANB2, bind to ssRNA comprising the core sequence GGU, such as ssRNA comprising one or more copies of the sequence NGGUNN and/or GGUA and/or AGGU and/or AGGUAA and/or polyuridine or poly(U) e.g., U₉. The binding affinity to ssRNA may be modified by providing a plurality of linked RanBP2-type zinc finger domains which may be the same or different, and preferably wherein at least two of the domains is spaced apart by a linker region. Alternatively, or in addition; a plurality of ssRNA substrate sequences in ssRNA may be employed e.g., tandem repeats of substrate sequences wherein each repeat is optionally separated by a ribonucleoside spacer comprising at least about 1-3 residues in length.

Accordingly, one example of the present invention provides a composition comprising at least one RanBP2-type zinc finger domain e.g., as defined herein above, or a variant or analog thereof capable of binding to single-stranded RNA (ssRNA), wherein said ssRNA comprises at least one occurrence of a sequence that binds to a RanBP2-type zinc finger domain or variant or analog thereof.

In one example, the composition is a peptide or polypeptide comprising at least one RanBP2-type zinc finger domain, variant or analog thereof. In another example, the composition is a peptide or polypeptide consisting essentially of at least one RanBP2-type zinc finger domain, variant or analog thereof. In another example, the composition is a peptide or polypeptide consisting of at least one RanBP2-type zinc finger domain, variant or analog thereof.

In another example, the composition is a polypeptide e.g., comprising the RanBP2-type zinc finger domain(s), variant(s) or analog(s) linked constrained within a peptidyl display scaffold. In one example, the display scaffold for the RanBP2-type zinc finger domain(s), variant(s) or analog(s) comprises a small antibody such as an immunoglobulin VH domain or immunoglobulin VL domain e.g., VH CDR1 fused non-contiguously to VL CDR3 or VHCDR1-VHFR2-VLCDR3. In another example, the display scaffold comprises a nanobody or single domain antibody e.g., the camelid VHH or the shark IgNAR single domain antibody or VNAR fragment. In another example, the display scaffold comprises a T-cell receptor polypeptide or fragment thereof. In another example, the display scaffold comprises a human I-set immunoglobulin domain. In another example, the display scaffold comprises a fibronectin domain e.g., FN3 lacking the immunoglobulin canonical inter-sheet disulphide bond. In another example, the display scaffold comprises a cystine knot miniprotein e.g., derived from a plant cyclotide. In another example, the display scaffold comprises a tetracorticopeptide (TPR). In another example, the display scaffold comprises a retinoid-X-receptor domain. In another example, the display scaffold comprises an armadillo repeat proteins. In another example, the display scaffold comprises a Designed Ankyrin Repeat Protein (DARPin). In another example, the display scaffold comprises a variable lymphocyte receptor (VLR) from lamprey. In another example, the display scaffold comprises an adnectin e.g., based on the FN3 domain. In another example, the display scaffold comprises an A domain e.g., derived from an extracellular cysteine-rich cell surface receptor protein or an avimer comprising multimers of human A domains. In another example, the display scaffold comprises an affibody derived from the Z domain of Staphylococcal protein A. In another example, the display scaffold comprises anticalin or lipocalin. In another example, the display scaffold comprises an intrabody targeted to an intracellular target.

In another example, the display scaffold is a β-turn scaffold comprising L-amino acids e.g., Cochran et al., J. Am. Chem. Soc., 123, 625-632 (2001). In another example, the scaffold comprises the pIII (syn. p3) or pVIII (syn. p8) protein of a filamentous phage such as M13. In yet another example, the scaffold comprises a serum protein moiety e.g., albumin or ferritin or transferrin or immunoglobulin or immunoglobulin fragment such as a domain antibody (dAb) or modified Fc component of immunoglobulin lacking effector function or Fc-disable immunoglobulin such as a CovXBody. In yet another example, the scaffold comprises a serum protein-binding moiety e.g., albumin-binding peptide, albumin-binding domain (ABD or Affybody) or serum albumin binding antibody domain (AlbudAb) that binds to albumin or immunoglobulin (Ig) or Ig fragment such as Fc or serum protein-binding moiety.

In another example, the scaffold comprises a protein transduction domain to facilitate intracellular or nuclear transport e.g., a protein transduction domain derived from HIV tat basic region, Kaposi fibroblast growth factor (FGF) hydrophobic peptide, transportan or Drosophila melanogaster penetratin, or a retroinverted analog thereof. The protein transduction domain may be coupled directly or indirectly to the RanBP2-type zinc finger domain(s) and/or variant(s) and/or analog(s) of the compositions, and they may be provided in the form of retro-peptide analogs or retroinverted peptide analogs.

In another example, the composition comprises the RanBP2-type zinc finger domain(s), variant(s) or analog(s) constrained within a non-peptidyl display scaffold e.g., wherein the peptidyl moiety and the non-peptidyl moiety are linked covalently. For example, the scaffold may comprise a carbohydrate display scaffold e.g., a sugar amino acid (SAA) e.g., a cyclic structure comprising glucosyluronic acid or an anomeric spiroannelated glycodiazepine molecular scaffold. Alternatively, the carbohydrate display scaffold may comprise a polycyclic variant e.g., pyranofuran or bicyclic sugar amino acid or spironucleoside. Alternatively, the carbohydrate display scaffold may comprise an iminosugar, e.g., 1-azafagomine or an analog of 1-azafagomine, pyrrolidine, piperidine. Alternatively, the carbohydrate display scaffold comprises a monosaccharide e.g., a tetrasubstituted xylofuranose. Alternatively, the carbohydrate display scaffold comprises a polymer such as hyaluronate or chitosan. In another example, the scaffold may comprise a nucleic acid display scaffold e.g., a nucleic acid aptamer, single-stranded DNA, single-stranded RNA, phage RNA, phage DNA, etc. In another example, the scaffold may comprise a small molecule display scaffold e.g., staurosporine or streptavidin or a toxin such as an antibiotic molecule. In further examples, the scaffold may comprise a nanoparticle, coloured latex, radionuclide, a hydrolysable polyethylene glycol (PEG), hydroxyethyl starch (HES) or polyglycine.

Accordingly, the present invention also provides a composition comprising at least one PEGylated RanBP2-type zinc finger domain, variant or analog. In another example, the present invention provides at least one HESylated RanBP2-type zinc finger domain, variant or analog. In another example, the present invention provides at least one polyglycinated RanBP2-type zinc finger domain, variant or analog. In another example, the present invention provides a composition comprising at least one RanBP2-type zinc finger domain, variant or analog as described according to any example hereof and a serum protein moiety. In another example, the present invention provides a composition comprising at least one RanBP2-type zinc finger domain, variant or analog as described according to any example hereof and a peptidyl serum protein-binding moiety. In another example, the present invention provides a composition comprising at least one RanBP2-type zinc finger domain, variant or analog as described according to any example hereof and a non-peptidyl serum protein-binding moiety e.g., a hapten that binds to an Fc-disabled antibody, polyethylene glycol, hydroxyethyl starch (HES), polyglycine, a 4,4-diphenylcyclohexyl moiety or 4-phenylbutanoic acid moiety.

In another example, the present invention provides a composition comprising at least one RanBP2-type zinc finger domain comprising Structural Formula II:

X₂₋₃-Za-X₀₋₁-W-X-C-X₂₋₄-C-X-Zb-X₂-Zc-X-Zd-Ze-X₂-C-Zf-X-C,

wherein each of X, Za, Zb, Zc, Zd, Ze and Zf is an amino acid, and wherein a side chain of any one or more of Za to Zf is functional to contact at least one residue of single-stranded RNA such that W intercalates between two residues of a sequence-specific binding site in single-stranded RNA (ssRNA), or a variant or analog thereof. This interaction may be mediated by electrostatic forces and/or hydrogen bonding, and at least one of amino acid side chains of any one of amino acid residues Za to Zf may contact with at least one of the residues of the sequence-specific binding site.

In one example, Za is selected from amino acid residues of the group consisting of D, T, S, N and A. Alternatively, or in addition, Zb is selected from amino acid residues of the group consisting of N, A, L, V, E, K, Y and F. Alternatively, or in addition, Zc is selected from amino acid residues of the group consisting of F, W, K, A, P, S, W Q.

Alternatively, or in addition, Zd is selected from amino acid residues of the group consisting of R, W, K, E, T, L, S and G, and preferably selected from the group consisting of R, K, E, T, L, S and G or wherein Zd is R or wherein Zd is K. Alternatively, or in addition, Ze is selected from amino acid residues of the group consisting of R, A, P, K, T, Q and N or from the group consisting of R, Q and N or wherein Ze is R or wherein Ze is Q or wherein Ze is N. Alternatively, or in addition, Zf is selected from N, F, V, T, and E.

For example, a RanBP2-type zinc finger domain according to any example hereof may comprise any one or more of Structural Formulae III to XVII hereof subject to the proviso that said RanBP2-type zinc finger domain binds ssRNA, wherein:

(i) Structural Formula III consists of:

X₃-D-W-X-C-X₂₋₄-C-X-Zb-X-N-F-X-Zd-R-X₂-C-Zf-X-C;

(ii) Structural Formula IV consists of:

X₃-D-W-X-C-X₂₋₄-C-X-Zb-X-N-W-X-Zd-R-X₂-C-Zf-X-C;

(iii) Structural Formula V consists of:

X₂₋₃-Za-X₀₋₁-W-X-C-X₂-C-X-Zb-X-N-Zc-P-Zd-Ze-X₂-C-Zf-X-C;

(iv) Structural Formula VI consists of:

X₂₋₃-Za-X₀₋₁-W-X-C-X₂-C-X-Zb-X-N-Zc-S-Zd-Ze-X₂-C-Zf-X-C;

(v) Structural Formula VII consists of:

F-X₃-D-W-X-C-X₄-C-X-Zb-X-N-Zc-A-Zd-Ze-X₂-C-Zf-X-C;

(vi) Structural Formula VIII consists of:

F-X-A-X-D-W-X-C-X₄-C-X-Zb-X-N-Zc-A-Zd-Ze-X₂-C-Zf-X-C;

(vii) Structural Formula IX consists of:

F-X-P-X-D-W-X-C-X₄-C-X-Zb-X-N-Zc-A-Zd-Ze-X₂-C-Zf-X-C;

(viii) Structural Formula X consists of:

F-X₃-D-W-X-C-X₄-C-X-Zb-X-N-W-A-Zd-Ze-X₂-C-Zf-X-C;

(ix) Structural Formula XI consists of:

F-X₃-D-W-X-C-X₄-C-X-Zb-X-N-F-A-Zd-Ze-X₂-C-Zf-X-C;

(x) Structural Formula XII consists of:

F-X₃-D-W-X-C-X₄-C-X-Zb-X-N-Zc-A-R-Ze-X₂-C-Zf-X-C;

(xi) Structural Formula XIII consists of:

F-X₃-D-W-X-C-X₄-C-X-Zb-X-N-Zc-A-K-Ze-X₂-C-Zf-X-C;

(xii) Structural Formula XIV consists of:

F-X₃-D-W-X-C-X₄-C-X-Zb-X-N-Zc-A-Zd-R-X₂-C-Zf-X-C;

(xiii) Structural Formula XV consists of:

F-X₃-D-W-X-C-X₄-C-X-Zb-X-N-Zc-A-Zd-N-X₂-C-Zf-X-C;

(xiv) Structural Formula XVI consists of:

F-X₃-D-W-X-C-X₄-C-X-Zb-X-N-Zc-A-Zd-Ze-X₂-C-N-X-C; and

(xv) Structural Formula XVII consists of:

F-X₃-D-W-X-C-X₄-C-X-Zb-X-N-Zc-A-Zd-Ze-X₂-C-L-X-C.

Values of Za-Zf in this example are as described herein for Structural Formula I and/or Structural Formula II.

The exemplified structural formulae of the RanBP2-type zinc finger domain of the invention may be categorized conveniently into three structurally-related larger classes. For example, a RanBP2-type zinc finger domain according to any example hereof may comprise a structure selected from Structural Formulae III and IV (class I) or Structural Formulae V and VI (class II) or Structural Formulae VII to XVII (class III).

In another example, a RanBP2-type zinc finger domain comprises a sequence having at least about 80% or 85% or 90% or 95% or 99% or 100% identity to a sequence set forth in any one of SEQ ID NOs: 1 to 21 hereof, subject to the proviso that said RanBP2-type zinc finger domain binds ssRNA, and wherein:

(i)
SEQ ID NO: 1 consists of the sequence:
SDGDWICPDKKCGNVNFARRTSCNRC;
(ii)
SEQ ID NO: 2 consists of the sequence:
SANDWQCKTCSNVNWARRSECNMC;
(iii)
SEQ ID NO: 3 consists of the sequence:
RAGDWKCPNPTCENMNFSWRNECNQC;
(iv)
SEQ ID NO: 4 consists of the sequence:
RAGDWQCPNPGCGNQNFAWRTECNQC;
(v)
SEQ ID NO: 5 consists of the sequence:
KSGDWVCPNPSCGNMNFARRNSCNQC;
(vi)
SEQ ID NO: 6 consists of the sequence:
RPGDWDCPWCNAVNFSRRDTCFDC;
(vii)
SEQ ID NO: 7 consists of the sequence:
KFEDWLCNKCCLNNFRKRLKCFRC;
(viii)
SEQ ID NO: 8 consists of the sequence:
INEDWLCNKCGVQNFKRREKCFKC;
(ix)
SEQ ID NO: 9 consists of the sequence:
VIGTWDCDTCLVQNKPEAIKCVAC;

(x)
SEQ ID NO: 10 consists of the sequence:
EGSWWHCNSCSLKNASTAKKCVSC;
(xi)
SEQ ID NO: 11 consists of the sequence:
LADYWKCTSCNEMNPPLPSHCNRC;
(xii)
SEQ ID NO: 12 consists of the sequence:
SEDEWQCTECKKFNSPSKRYC;
(xiii)
SEQ ID NO: 13 consists of the sequence:
NANKWSCHMCTYLNWPRAIRCTQC;
(xiv)
SEQ ID NO: 14 consists of the sequence:
TAAMWACQHCTFMNQPGTGHCEMC;
(xv)
SEQ ID NO: 15 consists of the sequence:
FSANDWQCKTCSNVNWARRSECNMC;
(xvi)
SEQ ID NO: 16 consists of the sequence:
FSANDWQCKTCGNVNWARRSECNMC;
(xvii)
SEQ ID NO: 17 consists of the sequence:
FSAEDWQCSKCANVNWARRQTCNMC;
(xviii)
SEQ ID NO: 18 consists of the sequence:
FAAEDWVCSKCGNVNWARRRTCNVC;
(xix)
SEQ ID NO: 19 consists of the sequence:
FAAEDWICSKCGNVNWARRKTCNVC;
(xx)
SEQ ID NO: 20 consists of the sequence:
FGPNDWPCPMCGNINWAKRMKCNIC;
and
(xxi)
SEQ ID NO: 21 consists of the sequence:
FRAGDWKCSTCTYHNFAKNVVCLRC.

In another example, a RanBP2-type zinc finger domain or a variant thereof comprises a sequence having at least about 80% or 85% or 90% or 95% or 99% or 100% identity to a sequence set forth in any one of SEQ ID NOs: 1, 2 or 4 to 21 hereof or is other than SEQ ID NO: 3, subject to the proviso that said RanBP2-type zinc finger domain binds ssRNA.

The sequence of a RanBP2-type zinc finger domain or variant or analog thereof according to any example hereof may comprise additional residues added to the N-terminus and/or at the C-terminus. In one example, a RanBP2-type zinc finger domain or variant thereof comprises an additional residue added to the C-terminus of any one of Structural Formulae I-XVII or any one of SEQ ID NOs: 1-21 hereof, wherein the residue is a naturally-occurring amino acid or an analog thereof e.g., a D-amino acid. In another example, a RanBP2-type zinc finger domain or variant thereof comprises an additional two residues added to the C-terminus of any one of Structural Formulae I-XVII or any one of SEQ ID NOs: 1-21 hereof, wherein each of said residues is a naturally-occurring amino acid or an analog thereof e.g., a D-amino acid. In another example, a RanBP2-type zinc finger domain or variant thereof comprises an additional three residues added to the C-terminus of any one of Structural Formulae I-XVII or any one of SEQ ID NOs: 1-21 hereof, wherein each of said residues is a naturally-occurring amino acid or an analog thereof e.g., a D-amino acid. In another example, a RanBP2-type zinc finger domain or variant thereof comprises an additional four residues added to the C-terminus of any one of Structural Formulae or any one of SEQ ID NOs: 1-21 hereof, wherein each of said residues is a naturally-occurring amino acid or an analog thereof e.g., a D-amino acid. In another example, a RanBP2-type zinc finger domain or variant thereof comprises an additional five residues added to the C-terminus of any one of Structural Formulae I-XVII or any one of SEQ ID NOs: 1-21 hereof, wherein each of said residues is a naturally-occurring amino acid or an analog thereof e.g., a D-amino acid. In another example, a RanBP2-type zinc finger domain of variant thereof comprises an additional six residues added to the C-terminus of any one of Structural Formulae I-XVII or any one of SEQ ID NOs: 1-21 hereof, wherein each of said residues is a naturally-occurring amino acid or an analog thereof e.g., a D-amino acid. In another example, a RanBP2-type zinc finger domain or variant thereof comprises an additional seven residues added to the C-terminus of any one of Structural Formulae I-XVII or any one of SEQ ID NOs: 1-21 hereof, wherein each of said residues is a naturally-occurring amino acid or an analog thereof e.g., a D-amino acid. An additional 1-7 residues added to the C-terminus of a RanBP2-type zinc finger domain or variant or analog thereof may enhance the ssRNA binding activity of the domain.

In a related example, a RanBP2-type zinc finger domain, variant or analog consists essentially of a polypeptide moiety comprising at least about 24 or 25 or 26 or 27 or 28 or 29 or 30 or 3.1 or 32 or 33 or 34 or 35 amino acids in length. For example, a RanBP2-type zinc finger domain or variant thereof may comprise at least about 24 to 35 amino acids in length having at least about 80% or about 81% or about 82% or about 83% or about 84%, or about 85% or about 86% or about 87% or about 88% or about 89% or about 90% or about 90% or about 91% or about 92% or about 93% or about 94% or about 95% or about 96% or about 97% or about 98% or about 99% identical to a sequence selected from any one of SEQ ID NOs: 1 to 21. Alternatively, a RanBP2-type zinc finger domain may comprise a sequence selected from SEQ ID NOs: 1 to 21 or a variant or analog thereof.

Exemplary variants of RanBP2-type zinc finger domains comprise a modified amino acid sequence relative to a “base” RanBP2-type zinc finger domain sequence e.g., set forth in any one of Structural Formulae I-XVII or any one of SEQ ID NOs: 1-21, wherein one or more amino acids of a RanBP2-type zinc finger domain forming an interface with the ssRNA substrate is modified to thereby enhance or modify substrate specificity. In one example, a variant RanBP2-type zinc finger domain comprises Structural Formula II wherein any one or more of Za, Zb, Zc, Zd, or Ze is modified. Preferred variants will comprise one or two or three or four or five or six amino acid substitutions relative to a base RanBP2-type zinc finger domain sequence. More generally, single substitutions are performed. For example, Zd and/or Ze is modified. For example, the present invention provides a variant RanBP2-type zinc finger domain wherein Zd is a basic amino acid e.g., arginine or lysine and/or wherein Ze is arginine or glutamine or asparagine. In these configurations, an arginine residue at Zd and/or Ze of the RanBP2-type zinc finger domain may coordinate with one or two guanine residues in the ssRNA substrate depending on the number of arginines at Zd and Ze, and/or a glutamine residue at Ze of in the RanBP2-type zinc finger domain may coordinate with adenine in the ssRNA substrate and/or an asparagine residue at Ze of in the RanBP2-type zinc finger domain may coordinate with uridine in the ssRNA substrate. Accordingly, such substitutions in a RanBP2-type zinc finger domain may modify substrate specificity as follows:

(i) the di-ribonucleotide GG when Zd and Ze are both arginine;
(ii) the di-ribonucleotide GA when Zd is arginine and Ze is glutamine;
(iii) the di-ribonucleotide GU when Zd is arginine and Ze is asparagine;
(iv) the di-ribonucleotide AG when Zd is glutamine and Ze is arginine; and
(v) the di-ribonucleotide AA when Zd and Ze are both glutamine.

Exemplary analogs of a RanBP2-type zinc finger domain have enhanced ssRNA binding activity and/or serum half-life compared to a corresponding base peptide from which it has been derived. Such analogs may comprise one or more D-amino acids e.g., an isostere, retro-peptide analog or retroinverted peptide analog of one or more base peptides and/or variants according to any example hereof and having ssRNA binding activity. Alternatively, or in addition, such an analog may comprise PEG i.e., it is PEGylated or hydroxyethyl starch (HES) i.e., it is HESylated, thereby enhancing serum half-life in an animal or human to which it has been administered. Alternatively, or in addition, such an analog may comprise polyglycine. For example, the present invention provides a PEGylated chiral analog of a RanBP2-type zinc finger domain as described according to any example hereof. In another example, the present invention provides a HESylated chiral analog of a RanBP2-type zinc finger domain as described according to any example hereof. In another example, the present invention provides a polyglycinated chiral analog of a RanBP2-type zinc finger domain as described according to any example hereof. In another example, the present invention provides a composition comprising a chiral analog of a RanBP2-type zinc finger domain as described according to any example hereof and a serum protein moiety as described according to any example hereof wherein the serum protein moiety may itself be a chiral analog such as comprising D-amino acids, or it may comprise L-amino acids. In another example, the present invention provides a composition comprising a chiral analog of a RanBP2-type zinc finger domain as described according to any example hereof and a peptidyl serum protein-binding moiety as described according to any example hereof, wherein the serum protein-binding moiety may itself be a chiral analog such as by comprising D-amino acids, or it may comprise L-amino acids. In another example, the present invention provides a composition comprising a chiral analog of a RanBP2-type zinc finger domain as described according to any example hereof and a non-peptidyl serum protein-binding moiety e.g., a hapten that binds to Fc, polyethylene glycol, hydroxyethyl starch (HES), polyglycine, a 4,4-diphenylcyclohexyl moiety or 4-phenylbutanoic acid moiety e.g., conjugated to D-lysine. Analogs may also be coupled to protein transduction domains, and such domains may themselves be presented in a form comprising D-amino acids, or they may be provided in the form of retro-peptide analogs or retroinverted peptide analogs.

In another example of the invention, one or more RanBP2-type zinc finger domains and/or variants and/or analogs according to any example hereof is fused to a detectable reporter molecule e.g., to produce a diagnostic reagent.

The compositions of the present invention according to any example hereof may comprise a plurality of RanBP2-type zinc finger domains and/or variants and/or analogs according to any example hereof. Such “multimeric” compositions generally have enhanced ssRNA binding activity and/or modify expression to a greater extent than the monomeric RanBP2-type zinc finger domains from which it is derived. For example, the effect of multimerization is more than the additive effect of either base peptide. Structurally, such multimeric compositions comprise two or more RanBP2-type zinc finger domains and/or variants and/or analogs, wherein each domain, variant or analog binds to a target site in ssRNA. Exemplary multimeric compositions of the invention comprise a plurality of covalently-linked RanBP2-type zinc finger domains, and/or variants and/or analogs e.g., two or three or four or five or six RanBP2-type zinc finger domains and/or variants and/or analogs. Two or more RanBP2-type zinc finger domains and/or variants and/or analogs of a multimeric composition may be linked contiguously or be adjacent to each other in a polypeptide. Alternatively, or in addition, at least two RanBP2-type zinc finger domains and/or variants and/or analogs of a multimeric composition may be linked non-contiguously i.e., they are spaced apart. To achieve such spacing, a linker molecule is generally employed e.g., comprising one or more glycine or serine residues, such as a polyglycine moiety or polyserine moiety.

Accordingly, the present invention also provides a composition comprising a plurality of RanBP2-type zinc finger domains and/or variants and/or analogs connected via optional linkers, wherein the plurality comprises Structural Formula XVIII:

X₁-L₁-X₂-[-L₂-X₃- . . . ]n

wherein:
X₁, X₂, and X₃ if present are each RanBP2-type zinc finger domains and/or variants and/or analogs according to any example hereof, wherein X₁, X₂, and X₃ may be the same or different;
L₁ and L₂ are each optional linker moieties wherein L₁ and L₂, if present, may be the same or different; and
n is zero or an integer, wherein when n is greater than 1 then each occurrence of X₃ may be the same or different to each other occurrence of X₃ and the same or different to X₁ and X₂, and each occurrence of L₂ may be the same or different to each other occurrence of L₂ and the same or different to L₁.

In one example, the present invention clearly encompasses multimeric compositions comprising repeats of the same RanBP2-type zinc finger domain and/or variant and/or analog, or alternatively, comprising different RanBP2-type zinc finger domains and/or variants and/or analogs. In accordance with this example, each of X₁, X₂, and X₃ may be the same or different. Similarly, each of X₁ and X₂, or each of X₁ and X₃ or each of X₂ and X₃ may be the same or different. Thus, the multimeric compositions may be homodimers or heterodimers, or higher-order molecules comprising repeats of one or more RanBP2-type zinc finger domains and/or variants and/or analogs with or without unique RanBP2-type zinc finger domains and/or variants and/or analogs.

Similarly, the multimeric compositions of the invention may include repeats of the same linker, and/or comprise different linkers. Thus, each of L₁ and L₂ may be the same or different. Similarly, each occurrence of L₂ may be the same or different, and different to L₁. In one example, L₁ is absent. In another example, at least one occurrence of L₂ is absent. In another example, L₁ and Lₐ are absent. In another example, L₁ and at least one occurrence of L₂ are both present. In another example, each RanBP2-type zinc finger domain and/or variant and/or analog is separated by a linker.

Wherein the linker is itself a peptidyl moiety, e.g., comprising serine or glycine or other L-amino acids or D-amino acids, the linker length and/or linker sequence may be optimized to thereby enhance specificity of binding to ssRNA and/or to enhance stability of a complex formed between (i) the RanBP2-type zinc finger domain(s) and/or variants and/or analogs and (ii) the ssRNA target or substrate. By virtue of the modular nature of zinc finger domains, each RanBP2-type zinc finger domain and/or variant and/or analog of a multimeric composition of the present invention targets a single site in ssRNA, permitting each RanBP2-type zinc finger domain, variant and analog to be selected based on the specific substrate recognition sites in ssRNA that are required to be targeted by the composition. These substrate recognition sites may be in the same ssRNA molecule, or different ssRNA molecules. Accordingly, the multimeric compositions of the invention may provide enhanced targeting of a single ssRNA species, or simultaneous targeting of different ssRNA species.

Exemplary inter-finger peptidyl linkers for use in such multimeric compositions will generally comprise up to about 30 amino acid residues in length, such as linker(s) consisting of 1 amino acid residue, or about 2 amino acid residues, or about 3 amino acid residues, or about 4 amino acid residues, or about 5 amino acid residues, or about 6 amino acid residues, or about 7 amino acid residues, or about 8 amino acid residues, or about 9 amino acid residues; or about 10 amino acid residues, or about 11 amino acid residues, or about 12 amino acid residues, or about 13 amino acid residues, or about 14 amino acid residues, or about 15 amino acid residues, or about 16 amino acid residues, or about 17 amino acid residues, or about 18 amino acid residues, or about 19 amino acid residues, or about 20 amino acid residues, or about 21 amino acid residues or about 22 amino acid residues, or about 23 amino acid residues, or about 24 amino acid residues, or about 25 amino acid residues, or about 26 amino acid residues, or about 27 amino acid residues, or about 28 amino acid residues, or about 29 amino acid residues, or about 30 amino acid residues. Conveniently, a peptidyl linker is about 5 to about 10 amino acids in length.

Alternatively, or in addition to modifying linker length, exemplary peptidyl linkers for use in multimeric compositions of the invention may comprise Structural Formula XIX:

M-K-Xₙ-G-L-F,

wherein:
X is any amino acid; and
n is zero or an integer having a value of less than 20.

Exemplary peptidyl linkers for use in multimeric compositions of the invention may comprise a sequence having at least about 80% identity to a sequence selected from SEQ ID NOs: 22-24, wherein:

(i)
SEQ ID NO: 22 consists of the sequence
-MKAGGTEAEKSRGLF;
(ii)
SEQ ID NO: 23 consists of the sequence
MKAGGSRGLF;
and
(iii)
SEQ ID NO: 24 consists of the sequence
MKGLF.

The percentage identity to any one of SEQ ID NOs: 22-24 may be about 80% or about 81% or about 82% or about 83% or about 84% or about 85% or about 86% or about 87% or about 88% or about 89% or about 90% or about 90% or about 91% or about 92% or about 93% or about 94% or about 95% or about 96% or about 97% or about 98% or about 99%. Alternatively, the linker may comprise a sequence having 100% identity to a sequence selected from SEQ ID NOs: 22-24.

As with peptidyl analogs of RanBP2-type zinc finger domains and variants, analogs of peptidyl linkers may be chiral analogs e.g., isosteres, retro peptide analogs or retroinverted peptide analogs of a linker that is exemplified herein by reference to an amino acid sequence.

A multimeric composition of the present invention may be PEGylated, HESylated, polyglycinated, multimerized, or comprise a serum protein moiety or a serum protein-binding moiety with or without intervening linker as described according to any example hereof, and may be a chiral analog according to any example hereof e.g., an isostere, retro peptide analog or retroinverted peptide analog.

The substrate sequence in ssRNA will generally comprise at least three ribonucleoside residues in length, and a sequence specific for a RanBP2-type zinc finger domain or a variant or analog thereof. The precise sequence of the substrate sequence may vary depending on the RanBP2-type zinc finger domain(s) and/or variant(s) and/or analog(s) employed.

In one example; the RanBP2-type zinc finger domain or a variant or analog thereof has specificity for a ssRNA substrate comprising at least one occurrence of the sequence GGU that binds to a RanBP2-type zinc finger domain or a variant or analog thereof. For example, the ssRNA substrate to which the composition of the present invention binds will comprise one or more additional ribonucleotides positioned at the 5′-end and/15 at the or 3′ end of the core sequence GGU. For example, the ssRNA substrate may comprise the core GGU sequence and an additional residue positioned at the 5′-end, which may be any ribonucleotide. Alternatively, or in addition, the ssRNA substrate may comprise the core GGU sequence and an additional residue positioned at the 3′-end, which may be any ribonucleotide. Alternatively, or in addition, the ssRNA substrate may comprise the core GGU sequence and two additional residues positioned at the 5′-end, each of which may be any ribonucleotide. Alternatively, or in addition, the ssRNA substrate may comprise the core GGU sequence and two additional residues positioned at the 3′-end, each of which may be any ribonucleotide. Alternatively, or in addition, the ssRNA substrate may comprise the core GGU sequence flanked by one or two additional residues positioned at the 5′-end and by one or two additional residues positioned at the 3′-end, each of which may be any ribonucleotide. For example, the ssRNA substrate may comprise a sequence selected from the group consisting of:
(i) the sequence NGGUNN, e.g., wherein N comprises adenosine such as in the sequence AGGUAA;
(ii) the sequence GGUN e.g., wherein N comprises adenosine such as in the sequence GGUA;
(iii) the sequence NGGU e.g., wherein N comprises adenosine such as in the sequence AGGU.

For example, the RanBP2-type zinc finger domain, variant or analog binds to a substrate sequence in ssRNA as exemplified in FIG. 7 hereof.

In another example, the RanBP2-type zinc finger domain or a variant or analog thereof has specificity for a ssRNA substrate comprising at least one occurrence of the sequence GAU that binds to a RanBP2-type zinc finger domain or a variant or analog thereof. For example, the ssRNA substrate to which the composition of the present invention binds will comprise one or more additional ribonucleotides positioned at the 5′-end and/at the or 3′ end of the core sequence GAU. For example, the ssRNA substrate may comprise the core GAU sequence and an additional residue positioned at the 5′-end, which may be any ribonucleotide. Alternatively, or in addition, the ssRNA substrate may comprise the core GAU sequence and an additional residue positioned at the 3′-end, which may be any ribonucleotide. Alternatively, or in addition, the ssRNA substrate may comprise the core GAU sequence and two additional residues positioned at the 5′-end, each of which may be any ribonucleotide. Alternatively, or in addition, the ssRNA substrate may comprise the core GAU sequence and two additional residues positioned at the 3′-end, each of which may be any ribonucleotide. Alternatively, or in addition, the ssRNA substrate may comprise the core GAU sequence flanked by one or two additional residues positioned at the 5′-end and by one or two additional residues positioned at the 3′-end, each of which may be any ribonucleotide. For example, the ssRNA substrate may comprise a sequence selected from the group consisting of
(i) the sequence NGAUNN, e.g., wherein N comprises adenosine such as in the sequence AGAUAA;
(ii) the sequence GAUN e.g., wherein N comprises adenosine such as in the sequence GAUA;
(iii) the sequence NGAU e.g., wherein N comprises adenosine such as in the sequence AGAU.

In another example, the RanBP2-type zinc finger domain or a variant or analog thereof has specificity for a ssRNA substrate comprising at least one occurrence of the sequence GUU that binds to a RanBP2-type zinc finger domain or a variant or analog thereof. For example, the ssRNA substrate to which the composition of the present invention binds will comprise one or more additional ribonucleotides positioned at the 5′-end and/at the or 3′ end of the core sequence GUU. For example, the ssRNA substrate may comprise the core GUU sequence and an additional residue positioned at the 5′-end, which may be any ribonucleotide. Alternatively, or in addition, the ssRNA substrate may comprise the core GUU sequence and an additional residue positioned at the 3′-end, which may be any ribonucleotide. Alternatively, or in addition, the ssRNA substrate may comprise the core GUU sequence and two additional residues positioned at the 5′-end, each of which may be any ribonucleotide. Alternatively, or in addition, the ssRNA substrate may comprise the core GUU sequence and two additional residues positioned at the 3′-end, each of which may be any ribonucleotide. Alternatively, or in addition, the ssRNA substrate may comprise the core GUU sequence flanked by one or two additional residues positioned at the 5′-end and by one or two additional residues positioned at the 3′-end, each of which may be any ribonucleotide. For example, the ssRNA substrate may comprise a sequence selected from the group consisting of:
(i) the sequence NGUUNN, e.g., wherein N comprises adenosine such as in the sequence AGUUAA;
(ii) the sequence GUUN e.g., wherein N comprises adenosine such as in the sequence GUUA;
(iii) the sequence NGUU e.g., wherein N comprises adenosine such as in the sequence AGUU.

In another example, the RanBP2-type zinc finger domain or a variant or analog thereof has specificity for a ssRNA substrate comprising at least one occurrence of the sequence AGU that binds to a RanBP2-type zinc finger domain or a variant or analog thereof. For example, the ssRNA substrate to which the composition of the present invention binds will comprise one or more additional ribonucleotides positioned at the 5′-end and/at the or 3′ end of the core sequence AGU. For example, the ssRNA substrate may comprise the core AGU sequence and an additional residue positioned at the 5′-end, which may be any ribonucleotide. Alternatively, or in addition, the ssRNA substrate may comprise the core AGU sequence and an additional residue positioned at the 3′-end, which may be any ribonucleotide. Alternatively, or in addition, the ssRNA substrate may comprise the core AGU sequence and two additional residues positioned at the 5′-end, each of which may be any ribonucleotide. Alternatively, or in addition, the ssRNA substrate may comprise the core AGU sequence and two additional residues positioned at the 3′-end, each of which may be any ribonucleotide. Alternatively, or in addition, the ssRNA substrate may comprise the core AGU sequence flanked by one or two additional residues positioned at the 5′-end and by one or two additional residues positioned at the 3′-end, each of which may be any ribonucleotide. For example, the ssRNA substrate may comprise a sequence selected from the group consisting of:
(i) the sequence NAGUNN, e.g., wherein N comprises adenosine such as in the sequence AAGUAA;
(ii) the sequence AGUN e.g., wherein N comprises adenosine such as in the sequence AGUA;
(iii) the sequence NAGU e.g., wherein N comprises adenosine such as in the sequence AAGU.

In another example, the RanBP2-type zinc finger domain or a variant or analog thereof has specificity for a ssRNA substrate comprising at least one occurrence of the sequence AAU that binds to a RanBP2-type zinc finger domain or a variant or analog thereof. For example, the ssRNA substrate to which the composition of the present invention binds will comprise one or more additional ribonucleotides positioned at the 5′-end and/at the or 3′ end of the core sequence AAU. For example, the ssRNA substrate may comprise the core AAU sequence and an additional residue positioned at the 5′-end, which may be any ribonucleotide. Alternatively, or in addition, the ssRNA substrate may comprise the core AAU sequence and an additional residue positioned at the 3′-end, which may be any ribonucleotide. Alternatively, or in addition, the ssRNA substrate may comprise the core AAU sequence and two additional residues positioned at the 5′-end, each of which may be any ribonucleotide. Alternatively, or in addition, the ssRNA substrate may comprise the core AAU sequence and two additional residues positioned at the 3′-end, each of which may be any ribonucleotide. Alternatively, or in addition, the ssRNA substrate may comprise the core AAU sequence flanked by one or two additional residues positioned at the 5′-end and by one or two additional residues positioned at the 3′-end, each of which may be any ribonucleotide. For example, the ssRNA substrate may comprise a sequence selected from the group consisting of:
(i) the sequence NAAUNN, e.g., wherein N comprises adenosine such as in the sequence AAAUAA;
(ii) the sequence AAUN e.g., wherein N comprises adenosine such as in the sequence AAUA;
(iii) the sequence NAAU e.g., wherein N comprises adenosine such as in the sequence AAAU.

In another example, the RanBP2-type zinc finger domain or a variant or analog thereof has specificity for a ssRNA substrate comprising at least one occurrence of a polyuridine sequence e.g., comprising up to about 9 or 10 uridine residues linked contiguously i.e., without other intervening residues.

The composition of the invention may have specificity for a ssRNA substrate comprising a plurality of ssRNA substrates according to any example hereof. Such a RanBP2-type zinc finger domain or a variant or analog may bind to a substrate comprising tandem copies of the same substrate sequence, or a plurality of copies of different sequences e.g., a combination of polyuridine and GGU motifs as described herein.

Generally, the number of RanBP2-type zinc finger domains and/or variants and/or analogs in a composition of the invention will not be fewer than the number of substrate sites being targeted, whether those substrate sites are in the same or different ssRNA molecules.

For example, a composition of the invention comprising two or more RanBP2-type zinc finger domains and/or variants and/or analogs thereof may bind to ssRNA comprising two or more contiguous substrate sequences.

Alternatively, a composition of the invention comprising two or more RanBP2-type zinc finger domains and/or variants and/or analogs thereof may bind to ssRNA comprising two or more non-contiguous substrate sequences. Any ribonucleotide may occur between substrate sites at which a RanBP2-type zinc finger domain and/or variant and/or analog docks e.g., as shown in FIG. 7 hereof.

The present invention also provides an isolated polypeptide comprising at least one RanBP2-type zinc finger domain or a variant or analog thereof capable of binding to single-stranded RNA (ssRNA) according to any example hereof, wherein the polypeptide is other than a naturally-occurring ZnF protein. In one example, such non-naturally occurring polypeptides provide an advantage over naturally-occurring ZnF proteins in being comprised of minimal RanBP2-type zinc finger domains and/or variants and/or analogs wherein each monomer is separated by a linker that is smaller than the inter-finger linkers present in the native protein, thereby facilitating protein display, and formulation and use of the polypeptide e.g., for the in vitro or in vivo or in situ modification of gene expression such as by modifying mRNA splicing. Alternatively, or in addition, such non-naturally occurring polypeptides provide an advantage over naturally-occurring ZnF proteins in being comprised of modular units of different RanBP2-type zinc finger domains and/or variants and/or analogs having different specificity to naturally-occurring ZnF proteins, thereby facilitating display of different ssRNAs and the use of the polypeptides for modifying expression of multiple different mRNAs e.g., in the same or different pathway, or for modifying expression of entire pathways.

In another example, the isolated polypeptide of the present invention comprises:
(i) any one of Structural Formulae I-XVII;
(ii) a functional fragment of (i);
(iii) a peptidyl fusion comprising a plurality of (i) and/or (ii) optionally wherein at least two of said plurality are separated by a linker molecule;
(iv) the structure, functional fragment or peptidyl fusion of any one of (i) to (iii) linked to a protein transduction domain, e.g., a HIV tat basic region or Kaposi FGF hydrophobic peptide protein or a retroinverted analog thereof and/or a serum protein-binding moiety, optionally wherein said peptide is separated from the protein transduction domain and/or serum protein-binding moiety by a spacer or said protein transduction domain and/or serum protein-binding moiety are separated by one or more spacers; and
(v) an analog of any one of (i) to (iv) selected from the group consisting of (a) the structure of any one of (i) to (iv) comprising one or more non-naturally-occurring amino acids; (b) the structure of any one of (i) to (iv) comprising one or more non-naturally-occurring amino acid analogs; (c) an isostere of any one of (i) to (iv); (d) a retro-peptide analog of any one of (i) to (iv); and (e) a retro-inverted peptide analog of any one of (i) to (iv).

In another example, the isolated polypeptide comprises a structure selected from the group consisting of:
(i) Structural Formula II:

X₂₋₃-Za-X₀₋₁-W-X-C-X₂₋₄-C-X-Zb-X₂-Zc-X-Zd-Ze-X₂-C-Zf-X-C;
wherein each of X, Za, Zb, Zc, Zd, Ze and Zf is an amino acid, and wherein a side chain of any one or more of Za to Zf is functional to contact at least one residue of single-stranded RNA such that W intercalates between two residues of a sequence-specific binding site in single-stranded RNA (ssRNA);
(ii) a functional fragment of (i);
(iii) a peptidyl fusion comprising a plurality of structures of said Structural Formula II and/or said functional fragments, optionally wherein at least two of said plurality are separated by a linker molecule;
(iv) any one of (i) or (ii) or (iii) additionally comprising a protein transduction domain or a retroinverted analog thereof and/or a serum protein-binding moiety, optionally wherein (i) or (ii) or (iii) is separated from the protein transduction domain and/or serum protein-binding moiety by a spacer or said protein transduction domain and/or serum protein-binding moiety are separated by one or more spacers; and
(v) an analog of any one of (i) to (iv) comprising one or more non-naturally-occurring amino acids or non-naturally-occurring amino acid analogs, or an isostere of any one of (i) to (iv), or a retro-peptide analog of any one of (i) to (iv), or a retro-inverted peptide analog of any one of (i) to (iv).

In another example, the isolated polypeptide of the present invention comprises a sequence selected individually or collectively from the group consisting of
(i) a sequence set forth in any one of SEQ ID NOs: 1 to 21, preferably any one of SEQ ID NOs: 1, 2 or 4-21 or preferably other than SEQ ID NO: 3;
(ii) the sequence of a functional fragment of any one of SEQ ID NOs: 1 to 21, preferably any one of SEQ ID NOs: 1, 2 or 4-21 or preferably other than SEQ ID NO: 3;
(iii) the sequence of a peptidyl fusion comprising a plurality of sequences at (i) and/or (ii), optionally wherein at least two of said plurality are separated by a linker molecule;
(iv) the sequence of (i) or (ii) or (iii) additionally comprising a protein transduction domain, e.g., a HIV tat basic region or Kaposi FGF hydrophobic peptide protein or a retroinverted analog thereof and/or a serum protein-binding moiety, optionally wherein said peptide is separated from the protein transduction domain and/or serum protein-binding moiety by a spacer or said protein transduction domain and/or serum protein-binding moiety are separated by one or more spacers; and
(v) an analog of any one of (i) to (iv) selected from the group consisting of (a) the sequence of any one of (i) to (iv) comprising one or more non-naturally-occurring amino acids; (b) the sequence of any one of (i) to (iv) comprising one or more non-naturally-occurring amino acid analogs; (c) an isostere of any one of (i) to (iv); (d) a retro-peptide analog of any one of (i) to (iv); and (e) a retro-inverted peptide analog of any one of (i) to (iv).

The present invention also provides a composition comprising a plurality of isolated RanBP2-type zinc finger domains and/or variants and/or analogs thereof or a plurality of isolated polypeptides comprising same as described according to any example hereof. In one example, a plurality of isolated RanBP2-type zinc finger domains and/or variants and/or analogs thereof or a plurality of isolated polypeptides comprising same is arrayed separately on a solid substrate e.g., microchip, a bead, a particle or a nanoparticle, such as a microchip, agarose, Sepharose (Pharmacia) or functionally-similar particle, a latex bead, or a nanoparticle. In one example, a plurality of isolated RanBP2-type zinc finger domains and/or variants and/or analogs thereof or a plurality of isolated polypeptides comprising same is combined in solution i.e., in admixture.

The present invention also provides an isolated polynucleotide other than a naturally-occurring ZnF protein-encoding gene, wherein the polynucleotide encodes one or more isolated RanBP2-type zinc finger domains and/or variants and/or analogs thereof or an isolated polypeptide comprising same as described according to any example hereof.

The polynucleotide of the invention may be provided in any suitable expression vector for expression in a host. Accordingly, a further example of the present invention provides a phagemid vector or cell capable of expressing a one or more isolated RanBP2-type zinc finger domains and/or variants and/or analogs thereof or an isolated polypeptide comprising same as described according to any example hereof. As will be understood by the skilled artisan, such an expression vector will generally encode naturally-occurring amino acids or otherwise be capable of being expressed by cellular translational machinery. Preferred expression vectors are selected from those having utility in human or other animal cells, or in plant cells.

In another example, a composition of the present invention is suitable for administration to a human or non-human animal. For example, the composition is formulated so as to comprise the active agent i.e., one or more RanBP2-type zinc finger domains and/or variants and/or analogs thereof, and a pharmaceutically acceptable carrier and/or excipient.

In one example, the composition is a liquid pharmaceutical formulation comprising a buffer in an amount to maintain the pH of the formulation in a range of about pH. 5.0 to about pH 7.0. In a further example, the pharmaceutical composition comprises an isotonizing agent in an amount to render same composition near isotonic. Exemplary isotonizing agents include sodium chloride e.g., present in said formulation at a concentration of about 50 mM to about 300 mM, or at a concentration of about 150 mM. Exemplary buffers are selected from the group consisting of succinate, citrate, and phosphate buffers e.g., at a concentration of about 1 mM to about 50 mM. For example, a sodium succinate or sodium citrate buffer at a concentration of about 5 mM to about 15 mM may be employed. In another example, the formulation further comprises a surfactant in an amount from about 0.001% to about 1.0% e.g., polysorbate 80 which may be present in said formulation in an amount from about 0.001% to about 0.5%.

Pharmaceutical compositions may be formulated for administration by injection, inhalation, ingestion or topically.

In one example, the formulation is for inhalation and the active agent is present in an amount suitable for administration by inhalation and the carrier or excipient is one suitable for inhalation. Inhalable formulations e.g., comprising an alkyl-saccharide transmucosal delivery-enhancing excipient such as Intraveil (Aegis Therapeutics) are preferred for prophylactic applications e.g., for administration to an asymptomatic subject at risk of developing a condition associated with inappropriate ssRNA expression or aberrant gene expression or a complication associated therewith e.g., an asymptomatic subject having one or more risk factors for a condition associated with inappropriate ssRNA expression or aberrant gene expression, and/or an asymptomatic subject exposed to a ssRNA viral agent that is a risk factor for development of a condition associated with viral ssRNA expression. By “asymptomatic subject” is meant a subject that does not exhibit one or more symptoms of a condition associated with inappropriate ssRNA expression or aberrant gene expression. By “inappropriate ssRNA expression” is meant ssRNA that occurs in a plant or animal subject (including a human) as a consequence of infection with a pathogen having a ssRNA genome or expressing ssRNA during infection, the expression of which is to be targeted. By “aberrant gene expression” is meant the expression of endogenous mRNA in a plant or animal subject (including a human) e.g., mRNA splice variant associated with a disease state, wherein the endogenous mRNA is associated with the presence of ssRNA e.g., miRNA or ncRNA.

In another example, the formulation is for injection and the active agent is present in an amount suitable for administration by injection e.g., subcutaneously, intravenously, intraperitoneally or intramuscularly, and the carrier or excipient is one suitable for injection e.g., subcutaneously, intravenously, intraperitoneally or intramuscularly.

The formulation may be packaged for multiple administrations e.g., it may be packaged as multiple injectable ampoules, capsules, etc. for repeated administration or repeated dosing.

The skilled artisan will be aware that an amount of the active ingredient will vary, e.g., as a result of variation in the bioactivity of the active agent, and/or the severity of the condition being treated. Accordingly, the term “amount” is not to be construed to limit the invention to a specific quantity, e.g., weight of active ingredient.

As used herein, the term “suitable carrier or excipient” shall be taken to mean a compound or mixture thereof that is suitable for use in a formulation albeit not necessarily limited in use to that context. In contrast, the term “a carrier or excipient” is compound or mixture thereof that is described in the art only with reference to a use in a formulation. The term “carrier or excipient for inhalation” shall be taken to mean a compound or mixture thereof that is suitable for use in a formulation to be administered to a subject by inhalation e.g., a formulation comprising an alkyl-saccharide transmucosal delivery-enhancing excipient such as Intraveil (Aegis Therapeutics). The term “carrier or excipient for injection” shall be taken to mean a compound or mixture thereof that is suitable for use in a formulation to be administered to a subject by injection.

A carrier or excipient useful in the formulation of the present invention will generally not inhibit to any significant degree a relevant biological activity of the active compound e.g., the carrier or excipient will not significantly inhibit the activity of the active compound with respect to binding to ssRNA(s) and/or modifying gene expression associated therewith. Alternatively, or in addition, the carrier or excipient comprises a compound that enhances uptake and/or delivery and/or efficacy of the active compound.

The carrier or excipient may comprise one or more protease inhibitors to thereby enhance the stability of a peptidyl moiety of the composition. Alternatively, or in addition, the carrier or excipient may comprise RNase to thereby facilitate degradation of the ssRNA to which the active agent binds. Alternatively, the carrier or excipient comprises an RNase inhibitor to thereby enhance the stability of ssRNA to which the active agent binds, optionally in combination with one or more protease inhibitors.

The present invention also provides a method for producing a formulation described according to any example hereof. For example, such a method comprises mixing or otherwise combining one or more isolated RanBP2-type zinc finger domains and/or variants and/or analogs thereof or an isolated polypeptide comprising same as described according to any example hereof in an amount sufficient to modify ssRNA expression with a suitable carrier or excipient e.g., a carrier or excipient for inhalation, ingestion or injection. In one example, the method additionally comprises producing or obtaining one or more isolated RanBP2-type zinc finger domains and/or variants and/or analogs thereof or an isolated polypeptide comprising same as described according to any example hereof. For example, one or more isolated RanBP2-type zinc finger domains and/or variants and/or analogs thereof or an isolated polypeptide comprising same is produced synthetically or recombinantly, using a method known in the art and/or described herein.

The composition of the invention is suitable for use in medicine e.g., in a method of treatment of the human or animal body by prophylaxis or therapy, or for use in research e.g., in a method of drug screening, drug development or clinical trial. For example, a composition of the invention according to any example hereof is for binding one or more isolated RanBP2-type zinc finger domains and/or variants and/or analogs thereof and a target ssRNA to thereby modify gene expression in agriculture, medicine or for research contexts. In another example, a composition of the invention according to any example hereof is for modulating the expression of viral ssRNA. In another example, a composition of the invention according to any example hereof is for modulating expression of mRNA splice variants associated with a disease state. In another example, a composition of the invention according to any example hereof is for use in a method of prophylaxis and/or therapy of one or more adverse effects or consequences of ssRNA expression, including viral ssRNA expression, miRNA expression or ncRNA expression. In a related example, the present invention provides for use of a composition of the invention according to any example hereof in medicine and/or in the preparation of a medicament for modulating gene expression associated with ssRNA levels in a cell.

The present invention also provides for use of a composition according to any example hereof to regulate or drive translation of specific mRNA targets. For example, the active agent of the composition may target the 5′-end of a specific mRNA and thereby recruit translational machinery sufficient to effect translation thereof. More particularly, a fusion comprising the translation factor eIF4G and one or more isolated RanBP2-type zinc finger domains and/or variants and/or analogs thereof targeted to a region 3′ of a CGG sequence in the 5′-UTR of the FMR1 gene may be employed to enhance translation of FMR1 mRNA e.g., in the treatment of Fragile X-associated tremor/ataxia syndrome (FXTAS) caused by mutations in the FMR1 gene that comprise expansions of the CGG sequence leading to reduced levels of FMR1 protein in the absence of reduced mRNA.

The present invention also provides for use of a composition according to any example hereof to modify splicing of one or more mRNA transcripts. In accordance with this example, targeted up-regulation or targeted down-regulation of splice variants encoding endogenous proteins is effected.

The present invention also provides for use of a composition according to any example hereof fused to a reporter molecule is employed as a diagnostic reagent e.g., to examine RNA localization.

The present invention also provides a method of preventing or treating one or more adverse consequences of ssRNA expression in a subject or in an isolated cell, said method comprising administering an amount of a composition of the invention according to any example hereof for a time and under conditions sufficient to bind to ssRNA and thereby modulate gene expression e.g., at the post-transcriptional level such as via mRNA processing.

### BRIEF DESCRIPTION OF DRAWINGS

FIG. 1 provides several representations demonstrating that ZRANB2 binds to ssRNA containing AGGUAA repeats. Panel A is a schematic representation showing substrate sequence variations comprising a conserved GGU tri-ribonucleotide repeat in the ssRNA substrate binding sites of ZRANB2-F12 after either 9 or 13 rounds of SELEX. Panel B is a photographic representation of a gel shift showing binding of ZRANB2 to ssRNA but not to dsDNA or dsRNA or a mutant ssRNA wherein the central GGU is replaced by CUG. Data indicate that the complex formation is selective towards ssRNA having GGU core sequence. Panel C is a graphical representation fluorescence anisotropy showing variable binding of a glutathione fusion with ZRANB2 (GST-F2) to a 17-nt ssRNA substrate sequence comprising a GGU core sequence and reduced binding to variants comprising point mutations within this core. Panel D is a graphical representation showing fluorescence anisotropy binding data for ZRANB2-F2 wherein association constants have been calculated for GST-F2 binding to a 17-nt ssRNA substrate oligonucleotide comprising single base mutations in and around the GGU core, and data represent the average association constant (Ka) for 3 experiments ±1 SD. Data show signficantly reduced association with ZRANB2 zinc finger domain when the GG di-ribonucleotide of the core is modified. Panel E is a graphical representation showing association constants obtained by fluorescence anisotropy for F12 binding to RNA sequences containing either a single AGGUAA site (with a scrambled second site) or double sites with spacings of −1, 0, 2, 5, and 8 adenines or the 5-nt sequence ACCCC (AC4). Panel F is a graphical representation showing association constants obtained by fluorescence anisotropy of RNA-binding affinity for single Ala point mutations of GST-F2, wherein data represent the average association constant (Ka) for 3 experiments ±1 SD.

FIG. 2 is a schematic representation showing an alignment of the two zinc finger domains of human ZRANB2, namely the F1 domain (amino acids 9-41) and F2 domain (amino acid 65-91) sequences. The asterisk denotes cysteine residues, and residues that directly contact RNA are marked by gray boxes. These interface residues correspond to positions D12, N22, F25, R27, R28 and N32 in the F1 domain of the full-length ZRANB2 protein, and to positions D68, N76, F79, R81, R82 and N86 in the F1 domain of the full-length ZRANB2 protein.

FIG. 3 provides representations showing NMR analysis of the ZRANB2-ssRNA interaction. Panel A is a schematic representation showing the structure of ZRANB2-F2 domain as determined by an overlay of the 20 lowest energy structures (residues 67-95), wherein N represents the N-terminus and C represents the C-terminus. Panel B is a space-filling representation of the F2 domain of ZRANB2, wherein the protein is rotated approximately 90° counter-clockwise about the vertical axis, compared with panel A, and residues having significant chemical-shift changes i.e., greater than 1 SD from the mean, are indicated. Panel C is a graphical representation showing an overlay of 15N-HSQC spectra of free F2 and F2 in the presence of 1.2 molar equivalents of CCAGGUAAAG (SEQ ID NO: 25), wherein arrows indicate shifts in selected resonances between the bound and unbound states.

FIG. 4 provides two-dimensional and three-dimensional representations showing structure of the ZRANB2:RNA complex. Panel A shows an electron density protein/RNA interface model wherein the binding interface is shown with unbiased Fₒ-F_{c} density at 2.50 as determined by omitting the ssRNA substrate. The final model for the RNA is shown to illustrate the fit with the difference density. 2Fₒ-F_{c} electron density (blue) calculated by using the final model is shown at 1.4 σ on the protein portion of the model only. Panel B shows an overview of the ZRANB2:RNA structure model, wherein the F2 domain is shown as a ribbon and the ssRNA ligand residues are shown as sticks and the zinc ion is shown as a sphere. The sequence Ade1-Gua2-Gua3-Ura4 i.e., AGGU of the ssRNA substrate is indicated. Interactions between Gua2 and R81 of the F2 domain and between Gua3 and R82 of the F2 domain are also shown, with the interpolation of W79 of the F2 domain there between. Panels C and D (Left) show ribbon and stick models depicting interactions between residues in the F2 domain and the GGU core of the ssRNA substrate, involving D68, W79, A80 and R81 on Gua 2, and V77, W79 and R82 on Gua3, and N76 and N86 on Ura4. Panels C and D (Right) show planar structures summarizing the hydrogen bonding of F2 domain residues to ssRNA comprising the GGU core.

FIG. 5 provides schematic representations of a 3-dimentional model showing the coordination of Ade5 and Ade6 in ssRNA around V77 and M87 of the F2 domain of ZRANB2. Panel A shows the structure of one conformer, wherein Ade5 and Ade6 were modeled with 50% occupancy. Residues 1-6 are indicated. Panel B shows the structure of a second conformer, wherein Ade5 points away from F2 and no density for Ade6 was observed. Panel C shows a model of the F2: ssRNA complex calculated by using the intermolecular NOEs between V77/M87 and Ade5 H2.

FIG. 6 provides representations showing a correlation between the ssRNA-binding preferences of ZRANB2 with its observed splicing activity. Panel A is a schematic representation showing exons 1-4 of a Tra2-3 minigene (boxed) and intervening intron sequences (lines) wherein the dominant transcript contains exons 1, 3, and 4. After the addition of ZRANB2, a transcript containing only exons 1 and 4 is observed (6). The sequences of the 5′-splice sites of each exon are shown as underlined sequences, wherein the vertical line indicates an intron-exon boundary. Panel B shows surface plasmon resonance data after injection of ZRANB2-F12 in the presence of a competitor RNA, onto a chip bearing the sequence of the 5′-splice site of exon 3. Data indicate that ZRANB2 mediates splicing of the Tra2-β minigene transcripts. Panel C shows sequence alignments of human RanBP2-like zinc finger domains indicating conservation of amino acid residues that mediate ssRNA recognition in ZRANB2.

FIG. 7 is a schematic representation showing alignments of substrate sequences in ssRNAs of 41 unique clone4s that bind to ZRANB2-F12 after either 9 or 13 rounds of SELEX. The sequences were aligned according to the most complete AGGUAA motif. Bases originating from the 25-nt randomized region are shown in uppercase letters and those from the flanking regions are shown in lowercase letters. The core AGGUAA motif sequences are indicated by shading. Inter0finger linkers are those residues between the shaded boxes.

FIG. 8 is a graphical representation showing fluorescence anisotropy binding data for ZRANB2 F1 domain. Association constants were calculated for GST-F1 binding to a 17-nt ssRNA oligonucleotide containing single base mutations. Data indicate the association constants for an average of 3 experiments ±1 SD.

FIG. 9A provides a graphical representation weighted average chemical-shift differences for the ZRANB2-F2 domain residues, wherein HN, H, N, C are shown as filled bars and side-chain atoms as open bars for unbound F2 and F2 bound to a single RNA site consisting of the sequence 5′-CCAGGUAAAG-3′ (SEQ ID NO: 25). The horizontal line indicates 1 SD above the average chemical-shift change.

FIG. 9B shows ¹H NMR spectra of F2 alanine mutants K72A, T73A, W79A, R81A, R82A, R87A and M87A, showing their correctly folding. Spectra were recorded at concentrations of 100-200 μM in 20 mM Tris/HCl (pH 8.0), 50 mM NaCl and 1 mM CaCl₂ at 25° C.

FIG. 9C shows intermolecular NOEs observed for the F2:RNA complex. A portion of a 2D NOESY recorded in D₂O is shown. Assignments are indicated.

FIG. 9D shows HSQC titration for F12 and a double site RNA. Overlay of 15N-HSQCspectra of free F12 and F12 in the presence of 1.2 molar equivalents of RNA consisting of the sequence 5′-AGGUAAAGGUAA-3′ (SEQ ID NO: 26). Arrows designate the shifting of selected resonances.

FIG. 10 provides schematic representations of X-ray data showing the interactions between ZRANB2 and ssRNA substrate. Panel A provides a stereoview of electron density at the protein/RNA interface. The final model is shown together with 2 Fₒ-F_{c} density. Panel B shows electron density in the F2: RNA structure. Left: A section of Fo electron density calculated by using initial phase information after density modification is shown at 1.6 σ, wherein the final model is shown to illustrate the relative fit to the density. Right: The same section from Panel A is shown with 2Fₒ-F_{c} density calculated after automated model building by the program Arp/Warp expert system shown at 1.3σ, wherein the final model is shown to illustrate the relative fit to the density. Panel C shows electron density for Ade1 and Ade6. Left: Fₒ-F_{c} difference density shown at 3.0 σ was calculated by omitting Ade1 from refinement and modelled at 50% occupancy wherein bases from the final model are shown to illustrate the fit with the unbiased difference density. Right: Fₒ-F_{c} difference density shown at 2.5 0 σ was calculated by omitting Ade6 from refinement at 50% occupancy wherein bases from the final model are shown to illustrate the fit with the unbiased difference density. Panel D shows the position of Y93 from a symmetry-related molecule of ZRANB2 in the crystal wherein the 2 different conformers of the M87 side chain are also shown.

FIG. 11A is a representation showing a sequence alignment of ZRANB2 F1 and F2 zinc finger domains Zinc-ligating cysteine residues are indicated by asterisks, constructs used for finger 1 (F1) and finger 2 (F2) domains are shown by lines, and residues determined to be important for binding RNA are highlighted in gray. All protein sequences start at amino acid position 1 in the full-length proteins except for the sequence from S. cerevisiae which starts at amino acid 336.

FIG. 11B shows partial 1D ¹H NMR spectra of the ZRANB2 F1 domain (700 μM) and F2 domain (200 μM) from C. elegans Y25C1A.8 (298 K, 600 MHz). F1 is clearly unfolded.

FIG. 12 is a tabular representation showing structural statistical parameters for the ensemble of 20 ZRANB2 F2 domain structures herein (above), and data refinement statistical parameters (below).

FIG. 13 is a schematic representation showing a deletion mutant series within the linker region between the F1 and F2 domains of ZRANB2. Linker sequences in each mutant are indicated. Deleted regions are also indicated by reference to the positions of deleted residues in the full-length ZRANB2 protein at the left of the drawing.

FIG. 14 is a graphical representation showing the association of specific deletion mutants indicated in FIG. 13 and wild-type F12 to ssRNA consisting of the sequence 5′-AAAGGUGGUAAAA-3′ (SEQ ID NO: 27).

FIG. 15 is a photographic representation of SDS-PAGE showing stability of specific deletion mutants indicated in FIG. 13 and wild-type F12.

FIG. 16 is a graphical representation showing the associations of ssRNA consisting of the sequence 5′-AAAGGUGGUAAAA-3′ (SEQ ID NO: 27) to a trimeric zinc finger polypeptide comprising a single F1 domain and two F2 domains of ZRANB2 in tandem (F122) and a dimeric zinc finger polypeptide comprising a single F1 domain and a single F2 domain of ZRANB2 in tandem with linker residues 45-64 deleted (F12 A45-64). Data indicate enhanced binding for the trimeric zinc finger polypeptide relative to the dimeric form.

FIG. 17 is a representation showing the structure of the ZRANB2 domain F2 zinc finger domain to ssRNA comprising the sequence AGGUAA. Positions of the Gua2 and Gua 3 substrate residues are shown. Tight associations between Gua-2 and R81 and between Gua3 and R82 are also indicated.

FIG. 18 is a planar representation showing hydrogen bonding between guanine in ssRNA and arginine (left) and between adenine in ssRNA and glutamine (right).

FIG. 19 is a graphical representation showing the effect of mutating R82 in the ZRANB2 F2 domain to glutamine on binding to substrate ssRNA comprising guanine at position 3 or adenine at position 3. Data indicate optimal binding of R82 to Gua3 in the substrate.

FIG. 20 is a graphical representation showing the affinity of binding of ZRANB2 F2 domain alanine mutants K72A, R81A and R82A (FIG. 9B) to a ssRNA substrate comprising the GGU core sequence. Data indicate the importance of the R81 and R82 residues un the interaction with this core sequence. Mutation of K72 did not abrogate binding to the core to the same degree as mutations in R81 and R82.

FIG. 21 is a representation showing a sequence alignment between ZRANB2 zinc finger domains of humans, rat, chicken. frog, firefly, C. elegans, C. brig, rice and yeast. Zinc-ligating cysteine residues are indicated by asterisks, and residues determined to be important for binding RNA are highlighted in gray. Data indicate a sub-class of RanBP2-type zinc finger domains.

FIG. 22 is a representation showing a vertical sequence alignment between human ZRANB2 zinc finger F1 and F2 domains and RanBP2-type zinc finger domains in other zinc finger proteins. Zinc-ligating cysteine residues are indicated by asterisks, and residues determined to be important for binding RNA are highlighted in gray. Data indicate sub-classes of RanBP2-type zinc finger domains.

FIG. 23 is a graphical representation showing binding of RanBP2-type zinc finger domains from the zinc finger proteins indicated on the x-axis to ssRNA comprising a GGU tri-ribonucleotide core sequence i.e., 5′-AGGUA-3′. Data indicate conserved substrate sequence specificity in ssRNA for RanBP2-type zinc finger domains.

FIG. 24 is a ribbon and stick representation showing a putative association of a generic RanBP2-type zinc finger domains to ssRNA comprising a GGU tri-ribonucleotide core sequence i.e., 5′-AGGUA-3′.

FIG. 25 provides ribbon and stick representations showing putative associations of a divergent RanBP2-type zinc finger domains to ssRNAs comprising different tri-ribonucleotide core sequences.

FIG. 26 provides a schematic representation showing display of RanBP2-type zinc finger domains on beads via GST fusions and their use to trap ssRNA comprising different tri-ribonucleotide core sequences in a biopanning protocol.

FIG. 27 provides a ribbon and stick representation showing putative associations of a multimeric composition of the invention comprising three RanBP2-type zinc finger domains e.g., F1-F2-F2 of ZRANB2, to ssRNA comprising repeats of different tri-ribonucleotide core sequences that bind the domains of the multimeric composition.

FIG. 28A is a schematic representation showing binding of: (i) a multimeric composition of the invention comprising two wild-type RanBP2-type zinc finger domains ZF1 and ZF2, separated by a linker region, to ssRNA comprising repeats of the GGU tri-ribonucleotide core sequence, wherein on binding the linker region forms a loop (above); and (ii) a multimeric composition of the invention comprising two wild-type RanBP2-type zinc finger domains ZF1 and ZF2, separated by a shortened linker region, to ssRNA comprising repeats of the GGU tri-ribonucleotide core sequence, wherein the linker region no longer forms a loop (below).

FIG. 28B is a photographic representation of SDS-PAGE showing stability of deletion mutants indicated in FIG. 28A, wherein the length of the inter-finger linker is indicated at the tope of this figure. Data indicate that RanBP2-type zinc finger domains separated by linkers of at least about 5 residues in length and/or up to 25 residues in length are stable.

FIG. 28C is a graphical representation showing the association of RanBP2-type zinc finger domains separated by linkers of 5, 10 or 25 residues in length to ssRNA consisting of the sequence 5′-AAAGGUGGUAAAA-3′ (SEQ ID NO: 27). Data indicate that RanBP2-type zinc finger domains separated by linkers of at least about 5 residues in length and/or up to 25 residues in length may bind ssRNA.

FIG. 29A is a schematic representation showing binding of: (i) a multimeric composition of the invention comprising two non-contiguous RanBP2-type zinc finger domains ZF1 and ZF2, separated by a linker region to ssRNA comprising three repeats of the GGU tri-ribonucleotide core sequence (above); and (ii) a multimeric composition of the invention comprising three non-contiguous RanBP2-type zinc finger domains ZF1 and ZF2 and ZF3 to ssRNA comprising three repeats of the GGU tri-ribonucleotide core sequence (below).

FIG. 29B is a graphical representation showing the associations of ssRNA comprising three repeats of the GGU tri-ribonucleotide core sequence to the constructs of FIG. 29A. Data indicate enhanced binding affinity of the trimeric zinc finger polypeptide to ssRNA relative to the dimeric form.

FIG. 30 is a representation showing the structure of the ZRANB2:RNA complex, wherein residues important for RNA binding are labelled and wherein the shaded area represents the space occupied by the ssRNA substrate and the ribbon and stick figure indicates a zinc finger domain of ZRANB2.

FIG. 31 is a schematic representation showing an alignment of the sequences of zinc finger domains of ZRANB2, EWS and RMB5 proteins. Boxes indicate ssRNA-binding residues.

FIG. 32A is a photographic representation showing a gel shift showing that a RanBP2-type zinc finger domain construct comprising the F1-F2 and F3 zinc finger domains of EKLF protein i.e., EKLF-F123 binds to ssRNA substrate, whereas a construct lacking the F1 domain does not bind to the same ssRNA substrate.

FIG. 32B is a graphical representation showing binding of a RanBP2-type zinc finger domain construct comprising the F1-F2 and F3 zinc finger domains of EKLF protein i.e., EKLF-F123 binds at higher affinity to ssRNA comprising poly(U) i.e., U₉, than to poly(C) or the sequence 5′-AGGUAA-3′.

FIG. 32C is a representation showing HSQC titration of a RanBP2-type zinc finger domain construct comprising the F1-F2 and F3 zinc finger domains of EKLF protein i.e., EKLF-F123 in the absence and presence of a ssRNA substrate comprising poly(U) i.e., U₉, showing the formation of a specific complex, wherein the EKLF:RNA spectrum is shown with a single contour.

FIG. 33 is a schematic representation showing that alternative splicing of mRNA is mediated by splicing factors. Panel A shows binding of RS proteins to ESE sites to promote exon inclusion, wherein exons are shown as blocks. Panel B shows binding of RG-rich proteins to ESS sites to promote exon skipping.

FIG. 34 is a schematic representation of a BiFC assay for monitoring RNA species in vivo, wherein binding of a first fusion protein comprising a RanBP2-type zinc finger domain fused to a first subunit of a reporter GFP and a second fusion protein comprising a RanBP2-type zinc finger domain fused to a second subunit of a reporter GFP each bind to a ssRNA substrate to thereby promote interaction between the first and second subunits of the reporter GFP and reconstitute the functional reporter GFP. In this scheme, the level of reporter activity is directly proportional to, or indicative of, the presence of the ssRNA substrate.

FIG. 35 is a graphical representation showing fluorescence anisotropy data for the interaction of the RanBP2-type zinc finger domain of the EWS ZnF protein with ssRNA comprising the sequence 5′-AGGUAA-3′.

FIG. 36 provides graphical representations showing binding affinities of the wild-type ZRANB2 F2 domain (left-hand panel, ZF: WT) and a modified ZRANB2 F2 domain having R82 substituted for asparagine (right-hand panel, ZF: R82N) to wild-type ssRNA substrate comprising the sequence AGGUAA (wt on the x-axis) and modified ssRNAs comprising adenine at position 2 of the wild-type sequence (2A on the x-axis; i.e., the sequence AAGUAA), or uridine at position 2 of the wild-type sequence (2U on the x-axis; i.e., the sequence AUGUAA), or adenine at position 3 of the wild-type sequence (3A on the x-axis; i.e., the sequence AGAUAA), or uridine at position 3 of the wild-type sequence (3A on the x-axis; i.e., the sequence AGUUAA). Data indicate that the modified R82N ZRANB2 F2 domain has a greater affinity of binding i.e., about 2-fold, to the modified ssRNA comprising uridine at position 3 compared to binding of the modified R82N ZRANB2 F2 domain to wild-type ssRNA substrate. Wild-type ZRANB2 F2 domain recognizes guanine at position 3 in the wild-type ssRNA substrate, and the amino acid substitution of guanine for asparagine at position 82 i.e., R82N, alters substrate preference from guanine to another nucleotide e.g., uridine. Overall, these date demonstrate an 8-fold change in binding affinity relative to the affinity of the wild-type ZRANB2 F2 domain for the wild-type and modified (3U) ssRNA substrate.

### GENERAL DESCRIPTION AND DEFINITIONS

The designation of nucleotide residues referred to herein are those recommended by the IUPAC-IUB, Biochemical Nomenclature Commission, wherein A represents Adenine, C represents Cytosine, G represents Guanine, T represents thymine, U represents uridine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein, which are intended for the purpose of exemplification only. Functionally-equivalent products, compositions and methods are clearly within the scope of the invention, as described herein.

### DETAILED DESCRIPTION OF EXEMPLARY EMBODIMENTS

### RanBP2-Type Zinc Finger Domains

The compositions as described herein according to any embodiment may comprise any one or more peptidyl RanBP2-type zinc finger domains and/or peptidyl or non-peptidyl analogs thereof and/or peptidyl or non-peptidyl variants thereof.

By “peptidyl” is meant a composition comprising covalently linked amino acids as an active agent. The amino acids may be L-amino acids or D-amino acids or a combination thereof.

By “non-peptidyl” is meant a composition having as an active agent a composition that does not comprise a sequence of amino acids having ssRNA binding activity.

### 1. Peptidyl RanBP2-Type Zinc Finger Domains and Variants and Analogs Thereof.

A peptidyl composition described herein may be a base peptide comprising one or more RanBP2-type zinc finger domains or variant or analog according to any example hereof, that functions in ssRNA binding.

The term “base peptide” refers to a peptide in an unmodified form that possesses a stated binding activity or modulatory activity.

The term “variant” or “analog” in the context of a RanBP2-type zinc finger domain refers broadly to a peptide in a modified form that possesses a stated modulatory activity or binding activity.

### Peptide Synthesis

A peptide or an analog or variant thereof is preferably synthesized using a chemical method known to the skilled artisan. For example, synthetic peptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

Generally, chemical synthesis methods comprise the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barmy and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis. Synthesis. Biology, Vol. 1, for classical solution synthesis. These methods are suitable for synthesis of a peptide of the present invention or an analog or variant thereof.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

A peptide, analog or variant as described herein can also be chemically prepared by other methods such as by the method of simultaneous multiple peptide synthesis. See, e.g., Houghten Proc. Natl. Acad. Sci. USA 82: 5131-5135, 1985 or U.S. Pat. No. 4,631,211.

As will be apparent to the skilled artisan based on the description herein, an analog or variant of a peptide of the invention may comprise D-amino acids, a combination of D- and L-amino acids, and various unnatural amino acids (e.g., α-methyl amino acids, Cα-methyl amino acids, and Nα-methyl amino acids, etc) to convey special properties. Synthetic amino acids include ornithine for lysine, fluorophenylalanine for phenylalanine, and norleucine for leucine or isoleucine. Methods for the synthesis of such peptides will be apparent to the skilled artisan based on the foregoing description.

### Recombinant Peptide Production

A peptide or analog or variant thereof or fusion protein may be produced as a recombinant protein. To facilitate the production of a recombinant peptide or fusion protein nucleic acid encoding same is preferably isolated or synthesized. Typically the nucleic acid encoding the recombinant protein is/are isolated using a known method, such as, for example, amplification (e.g., using PCR or splice overlap extension) or isolated from nucleic acid from an organism using one or more restriction enzymes or isolated from a library of nucleic acids. Methods for such isolation will be apparent to the ordinary skilled artisan and/or described in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

For expressing protein by recombinant means, a protein-encoding nucleic acid is placed in operable connection with a promoter or other regulatory sequence capable of regulating expression in a cell-free system or cellular system. For example, nucleic acid comprising a sequence that encodes a peptide is placed in operable connection with a suitable promoter and maintained in a suitable cell for a time and under conditions sufficient for expression to occur. Nucleic acid encoding a RanBP2-type zinc finger domain peptide is described herein or is derived from the publicly available amino acid sequence.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid (e.g., a transgene), e.g., in response to a developmental and/or external stimulus, or in a tissue specific manner. In the present context, the term “promoter” is also used to describe a recombinant, synthetic or fusion nucleic acid, or variant which confers, activates or enhances the expression of a nucleic acid (e.g., a transgene and/or a selectable marker gene and/or a detectable marker gene) to which it is operably linked. Preferred promoters can contain additional copies of one or more specific regulatory elements to further enhance expression and/or alter the spatial expression and/or temporal expression of said nucleic acid.

As used herein, the term “in operable connection with”, “in connection with” or “operably linked to” means positioning a promoter relative to a nucleic acid (e.g., a transgene) such that expression of the nucleic acid is controlled by the promoter. For example, a promoter is generally positioned 5′ (upstream) to the nucleic acid, the expression of which it controls. To construct heterologous promoter/nucleic acid combinations (e.g., promoter/nucleic acid encoding a peptide), it is generally preferred to position the promoter at a distance from the gene transcription start site that is approximately the same as the distance between that promoter and the nucleic acid it controls in its natural setting, i.e., the gene from which the promoter is derived. As is known in the art, some variation in this distance can be accommodated without loss of promoter function.

Should it be preferred that a peptide or fusion protein of the invention is expressed in vitro a suitable promoter includes, but is not limited to a T3 or a T7 bacteriophage promoter (Hanes and Plückthun Proc. Natl. Acad. Sci. USA, 94 4937-4942 1997).

Typical expression vectors for in vitro expression or cell-free expression have been described and include, but are not limited to the TNT T7 and TNT T3 systems (Promega), the pEXP1-DEST and pEXP2-DEST vectors (Invitrogen).

Typical promoters suitable for expression in bacterial cells include, but are not limited to, the lacz promoter, the Ipp promoter, temperature-sensitive λL or λR promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are well-known in the art and are described for example, in Ausubel et al (In: Current Protocols in Molecular Biology. Wiley Interscience, ISBN 047 150338, 1987), U.S. Pat. No. 5,763,239 (Diversa Corporation) and Sambrook et al (In: Molecular Cloning: Molecular Cloning: A Laboratory Manual, Cold Spring Harbor Laboratories, New York, Third Edition 2001).

Numerous expression vectors for expression of recombinant polypeptides in bacterial cells and efficient ribosome binding sites have been described, and include, for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen) the pBAD/TOPO or pBAD/thio—TOPO series of vectors containing an arabinose-inducible promoter (Invitrogen, Carlsbad, Calif.), the latter of which is designed to also produce fusion proteins with a Trx loop for conformational constraint of the expressed protein; the pFLEX series of expression vectors (Pfizer Inc., CT, USA); the pQE series of expression vectors (QIAGEN, CA, USA), or the pL series of expression vectors (Invitrogen), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (e.g., 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991).

A wide range of additional host/vector systems suitable for expressing a peptide or fusion protein of the present invention are available publicly, and described, for example, in Sambrook et al (In: Molecular cloning, A laboratory manual, second edition, Cold Spring Harbor Laboratory, Cold Spring Harbor, N.Y., 1989).

In other examples, the RanBP2-type zinc finger domain, especially any base peptide, is expressed on by phage display, cell display, or in vitro display:

For in vitro display, the expressed peptide is linked to the nucleic acid from which it was expressed such that said peptide is presented in the absence of a host cell. For example, the peptide is displayed by ribosome display, which directly links mRNA encoded by an expression construct to the peptide that it encodes. To display a nascent polypeptide in vitro, nucleic acid encoding it is cloned downstream of an appropriate promoter (e.g., bacteriophage T3 or T7 promoter) and a ribosome binding sequence, optionally including a translatable spacer nucleic acid (e.g., encoding amino acids 211-299 of gene III of filamentous phage M13 mp19) that stabilizes the expressed fusion protein within the ribosomal tunnel. Ribosome complexes are stabilized against dissociation from the peptide and/or its encoding mRNA by the addition of reagents such as, for example, magnesium acetate or chloroamphenicol.

For phage display, the expressed peptide is displayed on the surface of a bacteriophage, as described e.g., in U.S. Pat. No. 5,821,047 and U.S. Pat. No. 6,190,908. In general, nucleic acid comprising a sequence encoding the peptide is fused N-terminally or C-terminally to nucleic acid comprising a sequence encoding a phage coat protein e.g., M13 protein-3 (p3), M13 protein-7 (p7), or M13, protein-8 (p8).

In one example, a RanBP2-type zinc finger domain of the present invention is expressed C-terminally in a Fos fusion peptide i.e., as Fos-RanBP2-type zinc finger domain fusion in the phagemid vector pJuFo. The vector pJuFo also expresses p3 C-terminally in a c-Jun fusion peptide i.e., as c-Jun-p3. By virtue of the interaction between c-Jun and Fos, the RanBP2-type zinc finger domain of the present invention is displayed from pJuFo in trans as a dimer between the Fos-RanBP2-type zinc finger domain and c-Jun-p3 fusion peptides.

Alternatively, a RanBP2-type zinc finger domain of the present invention is expressed N-terminally as a p3 or p7 or p8 fusion peptide wherein the C-terminus of the peptide is fused to the N-terminus of p3 or p7 or p8. Nucleic acid encoding the RanBP2-type zinc finger domain is cloned into an insertion site in a suitable vector e.g., an EcoRI site or other restriction site, positioned such that the encoded RanBP2-type zinc finger domain is expressed as an in-frame fusion with the p3 or p7 or p8 protein.

A leader sequence e.g., PelB, comprising a translation start codon is generally positioned upstream of the insertion site. Preferably, the vector is configured so as to provide for expression of natural open reading frames in the introduced nucleic acid encoding the RanBP2-type zinc finger domain e.g., by ensuring the absence of intervening stop codons between the leader sequence and the p3 or p7 or p8 protein. The introduced nucleic acid may also be cloned in different reading frames to achieve this read-through.

Optionally, the RanBP2-type zinc finger domain-p3 or RanBP2-type zinc finger domain-p7 or RanBP2-type zinc finger domain-p8 fusion peptide is also a fusion with an intervening haemagglutinin (HA) tag moiety e.g., upstream of the p3/p7/p8 sequence and downstream of the RanBP2-type zinc finger domain in the fusion peptide.

The nucleic acid encoding the HA tag moiety is generally modified to remove the amber stop codon to thereby permit translational read-through from the 5′-end of sequence encoding the RanBP2-type zinc finger domain to the p3 or p7 or 8 moiety.

Optionally, the fusion peptide comprises a cysteine residue positioned e.g., at the N-terminus of the RanBP2-type zinc finger domain moiety or at the C-terminus of the RanBP2-type zinc finger domain moiety or at the N-terminus of the p3 or p7 or p8 moiety or at the N-terminus of a HA-p3 or HA-p7 or HA-p8 moiety.

The sequence encoding a fusion peptide according to any example hereof is displayed from an appropriate vector, e.g., a vector capable of replicating in bacterial cells. Suitable host cells e.g., E. coli, are then transformed with the recombinant vector. Said host cells are also infected with a helper phage particle encoding an unmodified form of the coat protein to which a nucleic acid fragment is operably linked. Transformed, infected host cells are cultured under conditions suitable for forming recombinant phagemid particles comprising more than one copy of the fusion protein on the surface of the particle. This system has been shown to be effective in the generation of virus particles such as, for example, a virus particle selected from the group comprising λ phage, T4 phage, M13 phage, T7 phage and baculovirus. Such phage display particles are then screened to identify a displayed protein having a conformation sufficient for binding to a target protein or nucleic acid.

Means for introducing the isolated nucleic acid molecule or a gene construct comprising same into a cell for expression are well-known to those skilled in the art. The technique used for a given organism depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using. DNA-coated tungsten or gold particles (Agracetus Inc., WI, USA) amongst others.

### Protein Transduction Domains

To facilitate entry into a cell, a RanBP2-type zinc finger domain or an analog or variant thereof or a polypeptide comprising same as described herein may be conjugated to a protein transduction domain, synthesized to include a protein transduction domain, or expressed recombinantly as a fusion protein comprising a protein transduction domain. As used herein, the term “protein transduction domain” shall be taken to mean a peptide or protein that is capable of enhancing, increasing or assisting penetration or uptake of a compound conjugated to the protein transduction domain into a cell either in vitro or in vivo. Those skilled in the art will be aware that synthetic or recombinant peptides can be delivered into cells through association with a protein transduction domain such as the TAT sequence from HIV or the Penetratin sequence derived from the Antennapaedia homeodomain protein (see, for example, Temsamani and Vidal, Drug Discovery Today 9: 1012-1019, 2004, for review).

A suitable protein transduction domain will be known to the skilled artisan and includes, for example, HIV-1 TAT fragment, signal sequence based peptide 1, signal sequence based peptide 2, transportan, amphiphilic model peptide, polyarginine, or a Kaposi fibroblast growth factor (FGF) hydrophobic peptide protein transduction domain. Additional suitable protein transduction domains are described, for example, in Zhao and Weisledder Medicinal Research Reviews, 24: 1-12, 2004 and Wagstaff and Jans, Current Medicinal Chemistry, 13: 1371-1387, 2006.

A protein transduction domain is covalently attached to the N-terminus or C-terminus of a RanBP2-type zinc finger domain of the present invention or an analog or variant thereof, and may be a chiral analog e.g., a retroinverso peptidyl moiety or PEGylated moiety. For example, a peptidyl fusion comprising a protein transduction domain positioned N-terminal to a RanBP2-type zinc finger domain of the present invention may be produced by standard peptide synthesis means or recombinant means without the exercise of undue experimentation based on the disclosure herein. Retroinverted peptide analogs comprising a protein transduction domain positioned N-terminal to a RanBP2-type zinc finger domain of the present invention, wherein the complete sequence is retroinverted are particularly preferred and produced without inventive effort based on the disclosure herein. Peptidyl fusions comprising a protein transduction domain and a RanBP2-type zinc finger domain of the present invention may comprise a spacer or linker moiety separating protein transduction domain and RanBP2-type zinc finger domain. Alternatively, the protein transduction domain and RanBP2-type zinc finger domain may be adjacent or juxtaposed in the peptidyl fusion.

### Serum Protein Moieties

As used herein, the term “serum protein moiety” shall be taken to refer to any serum protein, protein fragment or peptide having a long half life e.g., serum albumin, immunoglobulin, antibody fragment, transferrin, ferritin or other serum protein, having a long half life. By “long half life” is meant a half life in serum approximately the same as an albumin protein e.g., human serum albumin. In this respect, it is preferred for a serum protein moiety to confer on a RanBP2-type zinc finger domain of the present invention administered to a subject, including any base peptide or variant or analog thereof, a half-life that is at least about 25% or 50% or 75% or 90% or 95% or 99% the half-life of an endogenous serum albumin protein e.g., a murine animal or primate such as a human. For example, human serum albumin has a half life in humans of 19 days e.g., Peters et al., Adv. Protein Chem. 37, 161-245 (1985), and a half-life in mice of about 35 hours e.g., Chaudhury et al., J. Exp. Med. 197, 315-322 (2003).

A preferred serum protein moiety is an immunoglobulin fragment. By “immunoglobulin fragment” is meant any variant of an immunoglobulin wherein the undesired effector function of Fc has been disabled or deleted, and wherein the fragment has a long half life. For example, an immunoglobulin fragment may be an Fc-disabled antibody, immunoglobulin isotype not producing undesirable side-effects, or a modified Fc not producing undesirable Fc effector function. One preferred example of an Fc-disabled antibody is a CovXBody comprising a hapten linker and Fc-disabled antibody (CovX Research LLC, San Diego Calif. 92121, USA). The RanBP2-type zinc finger domain of the present invention may be linked to a CovXBody via the hapten linker moiety of the CovXBody according to the manufacturer's instructions.

A serum protein moiety is generally covalently attached to the N-terminus or C-terminus of a RanBP2-type zinc finger domain of the present invention. For example, a peptidyl fusion comprising a serum protein moiety positioned N-terminal or C-terminal to a RanBP2-type zinc finger domain of the present invention may be produced by standard peptide synthesis means or recombinant means without the exercise of undue experimentation based on the disclosure herein. Peptidyl fusions comprising a serum protein moiety and a RanBP2-type zinc finger domain of the present invention may comprise a spacer or linker moiety separating serum protein moiety and RanBP2-type zinc finger domain. Alternatively, the serum protein moiety and RanBP2-type zinc finger domain may be adjacent or juxtaposed in the peptidyl fusion.

Particularly preferred serum protein moieties for use in the present invention are retro-inverted peptides e.g., comprising a retroinverted analog of one or more serum protein moieties:

### Serum Protein-Binding Moieties

As used herein, the term “serum protein-binding moiety” shall be taken to refer to any peptide or protein having the ability to bind to a serum protein e.g., serum albumin or Fc region of an antibody or transferrin or ferritin or other serum protein having a long half life, to thereby enhance the half-life of a protein, especially a RanBP2-type zinc finger domain of the present invention. By “long half life” is meant a half life in serum approximately the same as an albumin protein e.g., human serum albumin. In this respect, it is preferred for a serum protein-binding moiety to confer on a RanBP2-type zinc finger domain of the present invention administered to a subject, including any base peptide or variant or analog thereof, a half-life that is at least about 25% or 50% or 75% or 90% or 95% or 99% the half-life of an endogenous serum albumin protein e.g., a murine animal or primate such as a human. For example, human serum albumin has a half life in humans of 19 days e.g., Peters et al., Adv. Protein Chem. 37, 161-245 (1985), and a half-life in mice of about 35 hours e.g., Chaudhury et al., J. Exp. Med. 197, 315-322 (2003).

Peptides and proteins that comprise an amino acid sequence capable of binding to serum albumin and increase the half-life of therapeutically relevant proteins and polypeptides are known in the art. Bacterial and synthetic serum protein-binding peptides are described e.g., in International Patent Publication Nos. WO1991/01743, WO2001/45746 and WO2002/076489. International Patent Publication No. WO2004/041865 describes “nanobodies” directed against serum albumin that can be linked to a protein to increase its half-life. Chaudhury et al., The J. Exp. Med. 3, 315-322 (2003) describe the neonatal Fc receptor (FcRn) or “Brambell receptor” as an pH-dependent serum protein-binding moiety. US Pat. Publication 20070269422 (Ablynx N.V.) discloses nanobodies or domain antibodies (dAbs) of about 115 amino acids in length and comprising framework regions i.e., FR1 to FR4 and complementarity-determining regions i.e., CDR1 to CDR3, and which have serum half-life of at least about 50% the natural half-life of serum albumin in a primate.

Preferred serum protein-binding moieties comprise peptides that consist of or comprise an albumin-binding domain (ABD) or albumin-binding domain antibody (dAb) e.g., as described by Nguyen et al., Protein Eng, Design Sel. 19, 291-297 (2006); Holt et al., Protein Eng, Design Sel. 21, 283-288 (2008); Johnsson et al., Protein Eng, Design Sel. 21, 515-527 (2008), and US Pat. Publication No. 20070202045 (Genentech, Inc.), each of which is incorporated herein by reference.

Particularly preferred peptidyl serum protein-binding moieties for use in the present invention are retro-inverted peptides e.g., comprising a retroinverted analog of one or more serum protein-binding peptidyl moieties described in US Pat. Publication No. 20070202045 or US Pat. Publication 20070269422.

Non-peptidyl serum protein-binding moieties include e.g., clofibrate, clofibric acid, Tolmetin, Fenoprofen, Diflunisal, Etodolac, Naproxen, Nambutone, Ibuprofen, Chlorothiazide, Gemfibrozil, Nalidixic Acid, Methyldopate, Ampicillin, Cefamandole Nafate, N-(2-Nitrophenyl)-anthranilic Acid, N-Phenylanthranilic Acid and Quinidine Gluconate. The RanBP2-type zinc finger domains of the present invention may also be myristoylated, and/or modified by addition of a 4,4-diphenylcyclohexyl moiety e.g., Kurtzhals et al., Biochem. J. 312 (1995); Zobel et al., Bioorg. Med. Chem. Lett. 13, 1513 (2003).

Particularly preferred non-peptidyl serum protein-binding moieties for use in the present invention include 4-phenylbutanoic acid moieties having hydrophobic substituents on the phenyl ring and conjugated to an amino acid such as a D-amino acid e.g., 4-(p-iodophenyl)butyric acid conjugated to D-lysine through the c-amino group e.g., Dumelin et al., Agnew. Chem. Int. Ed. 47, 3196-3201 (2008) incorporated herein by reference, and any one of a series of similar conjugates comprising 4-phenylbutanoic acid moieties. Free 4-(p-iodophenyl)butyric acid, or 4-(p-iodophenyl)butyric acid conjugated to D-lysine, is readily conjugated to a RanBP2-type zinc finger domain of the invention or an analog or variant thereof by condensation between hydrogen of an α-amino or c-amino group on the RanBP2-type zinc finger domain and the hydroxyl group of the 4-(p-iodophenyl)butyric acid moiety.

A serum protein-binding moiety is generally covalently attached to the N-terminus or C-terminus of a RanBP2-type zinc finger domain of the present invention or an analog or variant thereof, and may be a chiral analog e.g., a retroinverso peptidyl moiety or PEGylated moiety. For example, a peptidyl fusion comprising a serum protein-binding moiety positioned N-terminal or C-terminal to a RanBP2-type zinc finger domain of the present invention may be produced by standard peptide synthesis means or recombinant means without the exercise of undue experimentation based on the disclosure herein. Retroinverted peptide analogs comprising a serum protein-binding moiety positioned N-terminal or C-terminal to a RanBP2-type zinc finger domain of the present invention, wherein the complete sequence is retroinverted are particularly preferred and produced without inventive effort based on the disclosure herein. Other peptidomimetic strategies include e.g., peptoids, N-methylated peptides etc., which are also encompassed by the present invention. Peptidyl fusions comprising a serum protein-binding moiety and a RanBP2-type zinc finger domain of the present invention may comprise a spacer or linker moiety separating serum protein-binding moiety and RanBP2-type zinc finger domain. Alternatively, the serum protein-binding moiety and RanBP2-type zinc finger domain may be adjacent or juxtaposed in the peptidyl fusion. Such configurations are readily modified by the inclusion of a protein transduction domain as described herein.

Spacers and/or Linkers

Each of the components of a RanBP2-type zinc finger domain-containing construct e.g., a polypeptide comprising one or more RanBP2-type zinc finger domains or nor more analogs or variants thereof as described herein, and any protein transduction domain, PEG moiety, serum protein-binding moiety according to any example hereof, may optionally be separated by a spacer or linker moiety. The spacer or linker moiety facilitates the independent folding of each RanBP2-type zinc finger domain, and/or provides for an appropriate steric spacing between plural peptide components and between peptidyl and non-peptidyl components. A suitable linker will be apparent to the skilled artisan. For example, it is often unfavorable to have a linker sequence with high propensity to adopt α-helix or β-strand structures, which could limit the flexibility of the protein and consequently its functional activity. Rather, a more desirable linker is a sequence with a preference to adopt extended conformation. In practice, most currently designed linker sequences have a high content of glycine residues that force the linker to adopt loop conformation. Glycine is generally used in designed linkers because the absence of a β-carbon permits the polypeptide backbone to access dihedral angles that are energetically forbidden for other amino acids.

Preferably, the linker is hydrophilic, i.e. the residues in the linker are hydrophilic.

In another example, a linker is a glycine residue or polyglycine moiety or polyserin moiety. Linkers comprising glycine and/or serine have a high freedom degree for linking of two proteins, i.e., they enable the fused proteins to fold and produce functional proteins. Robinson and Sauer Proc. Natl. Acad. Sci. 95: 5929-5934, 1998 found that it is the composition of a linker peptide that is important for stability and folding of a fusion protein rather than a specific sequence.

In one example, linkers join identical peptide target binding moieties to form homodimers. In another example, linkers join different peptide target binding moieties to form heterodimers. In another example, the linker separates a RanBP2-type zinc finger domain of the invention from a protein transduction domain. In another example, the linker separates a RanBP2-type zinc finger domain of the invention from a PEG moiety. In another example, the linker separates a RanBP2-type zinc finger domain of the invention from a HES moiety. In another example, the linker separates a RanBP2-type zinc finger domain of the invention from a polyglycine moiety. In another example, the linker separates a RanBP2-type zinc finger domain of the invention from a serum protein moiety. In another example, the linker separates a RanBP2-type zinc finger domain of the invention from a serum protein-binding moiety. In another example, the linker separates a protein transduction domain from a PEG moiety, HED moiety, polyglycine moiety, serum protein moiety or serum protein-binding moiety.

Peptidyl linkers may also be derivatized or analogs prepared there from according to standard procedures described herein.

### Base Peptides

In one example, a base peptide comprises any one of Strctural Formulae I to XVII. In another example, a base peptide comprises an amino acid sequence set forth in any one of SEQ ID NOs: 1 to 21. In another example, a base peptide comprises an amino acid sequence set forth in any one of SEQ ID NOs: 1, 2 or 4 to 21 i.e., other than SEQ ID NO: 3.

### Peptide Variants

As used herein the term “variant” shall be taken to mean a peptide that is derived from a RanBP2-type zinc finger domain of the invention as described herein e.g., a fragment or processed form of the peptide, wherein the ssRNA binding activity of the base peptide is not abrogated e.g., a functional fragment. In this respect, the activity of a functional fragment need not equivalent to the activity of the base peptide (or an analog) from which it is derived. For example, the fragment may have slightly enhanced or reduced activity compared to the peptide or analog from which it is derived e.g., by virtue of the removal of flanking sequence.

The term “variant” also encompasses fusion proteins comprising a peptide of the invention. For example, the fusion protein comprises a label, such as, for example, an epitope, e.g., a FLAG epitope or a V5 epitope or an HA epitope. For example, the epitope is a FLAG epitope. Such a tag is useful for, for example, purifying the fusion protein. Alternatively, or in addition, a variant in this context may comprise a peptidyl protein transduction domain and/or serum protein-binding peptide or domain.

The term “variant” also encompasses a derivatized peptide, such as, for example, a peptide modified to contain one or more-chemical moieties other than an amino acid. The chemical moiety may be linked covalently to the peptide e.g., via an amino terminal amino acid residue, a carboxyl terminal amino acid residue, or at an internal amino acid residue. Such modifications include the addition of a protective or capping group on a reactive moiety in the peptide, addition of a detectable label, and other changes that do not adversely destroy the activity of the peptide compound. For example, a variant may comprise a PEG moiety, radionuclide, colored latex, etc.

A variant generally possesses or exhibits an improved characteristic e.g., enhanced protease resistance and/or longer half-life and/or enhanced transportability between cells or tissues of the human or animal body and/or reduced adverse effect(s) and/or enhanced affinity for ssRNA substrate.

The following examples of peptide variants may be employed separately or in combination using standard procedures known to the skilled artisan.

In one example, a peptide variant comprises a polyethylene glycol (PEG) moiety e.g., having a molecular mass of about 5 kDa or about 12 kDa or about 20 kDa or about 30 kDa or about 40 kDa. The PEG moiety may comprise a branched or unbranched molecule. A PEG moiety may be added to the N-terminus and/or to the C-terminus of a RanBP2-type zinc finger domain of the invention or a variant or analog thereof as described according to any example herein. A PEG moiety may enhance serum half-life of the RanBP2-type zinc finger domain e.g., by protecting the peptide from degradation. A PEG moiety may be separated from the N-terminus and/or C-terminus of the RanBP2-type zinc finger domain by a spacer e.g., comprising up to 6 or 7 or 8 or 9 or 10 carbon atoms such as an 8-amino-3,6-dioxaoctanoyl spacer. For example, a spacer may reduce steric hindrance of the interaction with ssRNA. Maleimide chemistry may be employed to conjugate a PEG moiety to the peptide e.g., via cysteine residues located either within or at the N-terminal end of the peptide. For peptides that are refractory to conjugation in this manner e.g., by virtue of intramolecular disulfide bridge formation, a variety of other chemistries known to the skilled artisan may be employed to ligate PEG moieties onto the N-terminal and/or C-terminal ends of the peptides.

In another example, a peptide variant comprises a hydroxyethyl starch (HES) moiety i.e., the RanBP2-type zinc finger domain is “HESylated”. The HES moiety may comprise a branched or unbranched molecule. A HES moiety may be added to the N-terminus and/or to the C-terminus of a RanBP2-type zinc finger domain of the invention, including a peptide, variant or analog thereof as described according to any example herein. A HES moiety may enhance serum half-life of the RanBP2-type zinc finger domain e.g., by protecting the peptide from degradation. A HES moiety may be separated from the N-terminus and/or C-terminus of the RanBP2-type zinc finger domain by a spacer e.g., comprising up to 6 or 7 or 8 or 9 or 10 carbon atoms such as an 8-amino-3,6-dioxaoctanoyl spacer. For example, a spacer may reduce steric hindrance of the interaction with ssRNA. Maleimide chemistry may be employed to conjugate a HES moiety to the peptide e.g., via cysteine residues located either within or at the N-terminal end of the peptide. For peptides that are refractory to conjugation in this manner e.g., by virtue of intramolecular disulfide bridge formation, a variety of other chemistries known to the skilled artisan may be employed to ligate HES moieties onto the N-terminal and/or C-terminal ends of the peptides.

In another example, a peptide variant comprises a polyglycine moiety e.g., comprising two or three or four or five or six or seven or eight or nine or ten glycine residues covalently linked. A polyglycine moiety may be added to the N-terminus and/or to the C-terminus of a RanBP2-type zinc finger domain of the invention, or a peptide, variant or analog thereof as described according to any example herein, to produce a “polyglycinated” peptide. A polyglycine moiety may enhance serum half-life of the RanBP2-type zinc finger domain e.g., by protecting the peptide from degradation. A polyglycine moiety may be further separated from the N-terminus and/or C-terminus of the RanBP2-type zinc finger domain by a spacer e.g., comprising up to 6 or 7 or 8 or 9 or 10 carbon atoms such as an 8-amino-3,6-dioxaoctanoyl spacer. Standard recombinant means, oxime chemistry or peptide synthetic means are employed to add a polyglycine moiety to a RanBP2-type zinc finger domain of the present invention. A polyglycine moiety may also be used in conjunction with another moiety to extend the half-life of a RanBP2-type zinc finger domain of the present invention as described according to any example hereof, wherein the polyglycine moiety itself may serve further as a spacer between the RanBP2-type zinc finger domain and the other moiety.

In another example, a peptide variant comprises a serum protein moiety or serum protein-binding moiety as described according to any example hereof, which may be added to the N-terminus and/or to the C-terminus of a RanBP2-type zinc finger domain of the invention or a variant or analog thereof. A serum protein moiety or serum protein-binding moiety may enhance serum half-life of the RanBP2-type zinc finger domain or translocation of the peptide in serum. A serum protein moiety or serum protein-binding moiety may be separated from the N-terminus and/or C-terminus of the RanBP2-type zinc finger domain by a spacer e.g., comprising up to 6 or 7 or 8 or 9 or 10 carbon atoms such as an 8-amino-3,6-dioxaoctanoyl spacer. For example, a spacer may reduce steric hindrance of the interaction with ssRNA.

In another example, the peptide variant comprises a plurality of peptides of the present invention. Such “chain-extended” variants may bind to ssRNA with higher affinity than the monomeric base peptide. Methods for producing multimeric proteins include conventional peptide synthesis and recombinant expression means.

It will be apparent to the skilled artisan that means for derivation of a peptide apply equally to any RanBP2-type zinc finger domain of the invention, an analog thereof, and any additional peptidyl components of a fusion peptide e.g., a protein transduction domain and/or peptidyl linker or spacer and/or serum protein moiety and/or serum protein-binding moiety to which the RanBP2-type zinc finger domain(s) and/or analog(s) is/are attached.

### Peptide Analogs

In another example of the invention, a peptide analog of a RanBP2-type zinc finger domain is employed.

As used herein, the term “analog” shall be taken to mean a peptide wherein the active portion is modified e.g., to comprise one or more naturally-occurring and/or non-naturally-occurring amino acids, provided that the peptide analogreatins ssRNA binding activity. For example, the term “analog” encompasses a peptide comprising one or more conservative amino acid changes relative to, a base peptide to which it is functionally analogous. In another example, an “analog” comprises one or more D-amino acids.

An analog generally possesses or exhibits an improved characteristic relative to a base peptide to which it is functionally analogous e.g., enhanced protease resistance and/or longer half-life and/or enhanced transportability between cells or tissues of plants or humans or animals and/or reduced adverse effect(s) and/or enhanced affinity for ssRNA.

Suitable peptide analogs include, for example, a peptide comprising one or more conservative amino acid substitutions. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

It also is contemplated that other sterically similar compounds may be formulated to mimic the key portions of the peptide structure. The generation of such an analog may be achieved by the techniques of modeling and chemical design known to those of skill in the art. It will be understood that all such sterically similar peptide analogs fall within the scope of the present invention.

An example of an analog of a peptide of the invention comprises one or more non-naturally occurring amino acids or amino acid analogs. For example, a RanBP2-type zinc finger domain as described herein comprises one or more naturally occurring non-genetically encoded L-amino acids, synthetic L-amino acids or D-enantiomers of an amino acid. For example, the peptide comprises only D-amino acids. For example, the analog comprises one or more residues selected from the group consisting of: hydroxyproline, β-alanine, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylananine 3-benzothienyl alanine 4-chlorophenylalanine, 2-fluorophenylalanine, 3-fluorophenylalanine, 4-fluorophenylalanine, penicillamine, 1,2,3,4-tetrahydro-tic isoquinoline-3-carboxylic acid 3-2-thienylalanine, methionine sulfoxide, homoarginine, N-acetyl lysine, 2,4-diamino butyric acid, p-aminophenylalanine, N-methylvaline, homocysteine, homoserine, ε-amino hexanoic acid, δ-amino valeric acid, 2,3-diaminobutyric acid and mixtures thereof.

Other amino acid residues that are useful for making the peptides and peptide analogs described herein can be found, e.g., in Fasman, 1989, CRC Practical Handbook of Biochemistry and Molecular Biology, CRC Press, Inc., and the references cited therein.

The present invention additionally encompasses an isostere of a peptide described herein. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, yr indicates the absence of an amide bond. The structure that replaces the amide group is specified within the brackets.

Other modifications include, for example, an N-alkyl (or aryl) substitution (ψ[CONR]), or backbone crosslinking to construct lactams and other cyclic structures. Other variants of the modulator compounds of the invention include C-terminal hydroxymethyl variants, O-modified variants (e.g., C-terminal hydroxymethyl benzyl ether), N-terminally modified variants including substituted amides such as alkylamides and hydrazides.

In another example, a peptide analog is a retro-peptide analog (see, for example, Goodman et al., Accounts of Chemical Research, 12:1-7, 1979). A retro-peptide analog comprises a reversed amino acid sequence of a RanBP2-type zinc finger domain sequence described herein. For example, a retro-peptide analog of a RanBP2-type zinc finger domain comprises a reversed structure of any one of Structural Formulae I to XVII. In another example, a retro-peptide analog of a RanBP2-type zinc finger domain comprises a reversed amino acid sequence of a sequence set forth in any one of SEQ ID NOs: 1 to 21. Optionally, the peptide analog comprises an additional feature, such as, for example, a protein transduction domain and/or serum protein moiety and/or serum protein-binding moiety, each of which may also be a retro-peptide analog. The retro-peptide analog according to any example hereof may be PEGylated.

In a further example, an analog of a peptide described herein is a retro-inverso peptide (as described, for example, in Sela and Zisman, FASEB J. 11:449, 1997). Evolution has ensured the almost exclusive occurrence of L-amino acids in naturally occurring proteins. As a consequence, virtually all proteases cleave peptide bonds between adjacent L-amino acids. Accordingly, artificial proteins or peptides composed of D-amino acids are preferably resistant to proteolytic breakdown. Retro-inverso peptide analogs are isomers of linear peptides in which the direction of the amino acid sequence is reversed (retro) and the chirality, D- or L-, of one or more amino acids therein is inverted (inverso) e.g., using D-amino acids rather than L-amino acids, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. An advantage of retro-inverso peptides is their enhanced activity in vivo due to improved resistance to proteolytic degradation, i.e., the peptide has enhanced stability. (e.g., Chorev et al., Trends Biotech. 13, 438-445, 1995).

Retro-inverso peptide analogs may be complete or partial. Complete retro-inverso peptides are those in which a complete sequence of a peptide descried herein is reversed and the chirality of each amino acid in a sequence is inverted, other than glycine, because glycine does not have a chiral analog. Partial retro-inverso peptide analogs are those in which only some of the peptide bonds are reversed and the chirality of only those amino acid residues in the reversed portion is inverted. The present invention clearly encompasses both partial and complete retro-inverso peptide analogs.

In this respect, such a retroinverso peptide analog may optionally include an additional component, such as, for example, a protein transduction domain, which may also be retroinverted. The retro-inverso peptide analog according to any example hereof may also be PEGylated, HESylated or polyglycinated.

In yet another example, a base peptide is mutated to thereby improve the bioactivity of the peptide, e.g., the affinity with which the peptide binds to a target molecule and/or the specificity with which a peptide binds to a target molecule. Methods for mutating a peptide will be apparent to the skilled artisan and/or are described herein an include e.g., affinity maturation. For example, diverse amino acid sequences may be derived from a base peptide and peptides produced, by synthetic or recombinant means. For affinity maturation employing synthetic means, the amino acid sequence of a RanBP2-type zinc finger domain is modified in silico e.g., so as to retain secondary structure characteristics of the base peptide, a data set of related sequences is produced, and the peptides are synthesized and screened for activity.

For affinity maturation employing recombinant means, it is necessary to mutate nucleic acids encoding a diverse set of amino acid sequences by site-directed or random mutagenesis approaches. For example, nucleic acid may be amplified using mutagenic PCR such as by (i) performing the PCR reaction in the presence of manganese; and/or (ii) performing the PCR in the presence of a concentration of dNTPs sufficient to result in misincorporation of nucleotides. Methods of inducing random mutations using PCR are known in the art and are described, for example, in Dieffenbach (ed) and Dveksler (ed) (In: PCR Primer: A Laboratory Manual, Cold Spring Harbour Laboratories, NY, 1995). Furthermore, commercially available kits for use in mutagenic PCR are obtainable, such as, for example, the Diversify PCR Random Mutagenesis Kit (Clontech) or the GeneMorph Random Mutagenesis Kit (Stratagene). For example, a PCR reaction is performed in the presence of at least about 200 μM manganese or a salt thereof, more preferably at least about 300 μM manganese or a salt thereof, or even more preferably at least about 500 μM or at least about 600 μM manganese or a salt thereof. Such concentrations manganese ion or a manganese salt induce from about 2 mutations per 1000 base pairs (bp) to about 10 mutations every 1000 bp of amplified nucleic acid (Leung et al Technique 1, 11-15, 1989).

Alternatively, nucleic acid is mutated by inserting said nucleic acid into a host cell that is capable of mutating nucleic acid. Such host cells are deficient in one or more enzymes, such as, for example, one or more recombination or DNA repair enzymes, thereby enhancing the rate of mutation to a rate that is rate approximately 5,000 to 10,000 times higher than for non-mutant cells. Strains particularly useful for the mutation of nucleic acids carry alleles that modify or inactivate components of the mismatch repair pathway. Examples of such alleles include alleles selected from the group consisting of mutY, mutM, mutD, mutT, mutA, mutC and mutS. Bacterial cells that carry alleles that modify or inactivate components of the mismatch repair pathway are known in the art, such as, for example the XL-1Red, XL-mutS and XL-mutS-Kan^{r} bacterial cells (Stratagene).

It will also be apparent to the skilled artisan that unitary analogs may be produced from any RanBP2-type zinc finger domain of the invention or a variant thereof, with or without any other peptidyl moieties e.g., as an analog of a fusion peptide comprising e.g., one or more RanBP2-type zinc finger domains and an element selected from a protein transduction domain and/or peptidyl linker or spacer and/or serum protein moiety and/or serum protein-binding peptide moiety to which the RanBP2-type zinc finger domain(s) is/are attached. Such unitary analogs may be derivatized as described herein.

### 2. Non-Peptidyl Analogs

A non-peptidyl analog may be a nucleic acid or small molecule or a variant or analog thereof according to any example hereof, that functions in binding ssRNA. Preferred non-peptidyl analogs are functional equivalents of a RanBP2-type zinc finger domain of the present invention, however they preferably possess modified activity or affinity for ssRNA or enhanced pharmaceutical properties e.g., longer half-life, enhanced uptake and/or transportability between cells or tissues of the animal body and/or suitability for a particular mode of administration e.g., injectability, inhalability or modified solubility characteristic.

In one example, a non-peptidyl analog is a small molecule. A suitable small molecule is identified from a library of small molecules. Techniques for synthesizing small organic compounds will vary considerably depending upon the compound, however such methods will be well known to those skilled in the art. In one embodiment, informatics is used to select suitable chemical building blocks from known compounds, for producing a combinatorial library. For example, QSAR (Quantitative Structure Activity Relationship) modeling approach uses linear regressions or regression trees of compound structures to determine suitability. The software of the Chemical Computing Group, Inc. (Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and logP (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, lead compounds identified in initial screens, can be used to expand the list of compounds being screened to thereby identify highly active compounds.

### Assays to Identify and Isolate Therapeutic and Prophylactic Compounds

Any assay described herein for identifying binding activity of a RanBP2-type zinc finger domain of the present invention or an analog or variant thereof to ssRNA, and/or an interaction between ssRNA and a RanBP2-type zinc finger domain of the present invention or an analog or variant thereof, may be employed to identify therapeutic and prophylactic compounds. In one example, molecules that modify the interaction between ssRNA and a RanBP2-type zinc finger domain of the present invention or an analog or variant thereof are identified. In another example, molecules that modify the conformation of a RanBP2-type zinc finger domain of the present invention or an analog or variant thereof are identified.

It is to be understood that art-recognized screens can be utilized in separately or collectively and in any order determined empirically to identify or isolate the desired product at a level of purity and having a suitable activity ascribed to it e.g., for therapy. The activity and purity of the compounds determined by these assays make the compound suitable of formulations e.g., injectable and/or inhalable medicaments and/or oral formulations for treatment and/or prophylaxis.

The present invention encompasses the use of any in silico or in vitro analytical method and/or industrial process for carrying out a screening method into a pilot scale production or industrial scale production of a compound identified in such screens.

### Formulations

The present invention provides for the use of a composition of the present invention as described according to any example hereof in the preparation of a medicament for treatment of a subject in need thereof e.g., for attenuation or alleviation or amelioration of an inappropriate ssRNA expression or aberrant gene expression in a cell, tissue, organ or whole organism.

A peptidyl or non-peptidyl composition of the invention as described herein according to any embodiment is formulated for therapy or prophylaxis with a carrier or excipient e.g., suitable for inhalation or injection.

The term “carrier or excipient” as used herein, refers to a carrier or excipient that is conventionally used in the art to facilitate the storage, administration, and/or the biological activity of an active compound. A carrier may also reduce any undesirable side effects of the active compound. A suitable carrier is, for example, stable, e.g., incapable of reacting with other ingredients in the formulation. In one example, the carrier does not produce significant local or systemic adverse effect in recipients at the dosages and concentrations employed for treatment. Such carriers and excipients are generally known in the art. Suitable carriers for this invention include those conventionally used, e.g., water, saline, aqueous dextrose, dimethyl sulfoxide (DMSO), and glycols are preferred liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, one or more alkylsaccharides, and the like.

The formulations can be subjected to conventional pharmaceutical expedients, such as sterilization, and can contain a conventional pharmaceutical additive, such as a preservative and/or a stabilizing agent and/or a wetting agent and/or an emulsifying agent and/or a salt for adjusting osmotic pressure and/or a buffer and/or other additives known in the art. Other acceptable components in the composition of the invention include, but are not limited to, isotonicity-modifying agents such as water and/or saline and/or a buffer including phosphate, citrate, succinate, acetic acid, or other organic acids or their salts.

In an example, a formulation includes one or more stabilizers, reducing agents, anti-oxidants and/or anti-oxidant chelating agents. The use of buffers, stabilizers, reducing agents, anti-oxidants and chelating agents in the preparation of compositions, is known in the art and described, for example, in Wang et al. J. Parent. Drug Assn. 34:452-462, 1980; Wang et al. J. Parent. Sci. Tech. 42:S4-S26 (Supplement), 1988. Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5.

Where the carrier is in powdered form, it is preferred that the carrier is also within an acceptable non-toxic pH range.

In another example, a formulation as described herein according to any embodiment additionally comprises a compound that enhances or facilitates uptake of a compound. Suitable dermal permeation enhancers are, for example, a lipid disrupting agent (LDA), a solubility enhancer, or a surfactant.

LDAs are typically fatty acid-like molecules proposed to fluidize lipids in the human skin membrane. Suitable LDAs are described, for example, in Francoeur et al., Pharm. Res., 7: 621-627, 1990 and U.S. Pat. No. 5,503,843. For example, a suitable LDA is a long hydrocarbon chain with a cis-unsaturated carbon-carbon double bond. These molecules have been shown to increase the fluidity of the lipids, thereby increasing drug transport. For example, oleic acid, oleyl alcohol, decanoic acid, and butene diol are useful LDAs.

Solubility enhancers act by increasing the maximum concentration of drug in a composition, thus creating a larger concentration gradient for diffusion. For example, a lipophilic vehicle isopropyl myristate (IPM) or an organic solvent ethanol or N-methyl pyrrolidone (NMP) or dimethyl sulfoxide (DMSO) are suitable solubility enhancers (Liu et al., Pharm. Res. 8: 938-944, 1991; and Yoneto et al., J. Pharm. Sci. 84: 853-860, 1995).

Surfactants are amphiphilic molecules capable of interacting with the polar and lipid groups in the skin. These molecules have affinity to both hydrophilic and hydrophobic groups, which facilitate in traversing complex regions of the dermis. Suitable surfactants include, for example, an anionic surfactant lauryl sulfate (SDS) or a nonionic surfactant polysorbate 80 (Tween 80). Suitable surfactants are described, for example, in Sarpotdar et al., J. Pharm. Sci. 75: 176-181, 1986)

In another example, the formulation is a microemulsion. Microemulsion systems are useful for enhancing transdermal delivery of a compound. Characteristics of such microemulsion systems are sub-micron droplet size, thermodynamic stability, optical transparency, and solubility of both hydrophilic and hydrophobic components. Microemulsion systems have been shown to be useful for transdermal delivery of compounds and to exhibit improved solubility of hydrophobic drugs as well as sustained release profiles (Lawrence, et. al. Int. Journal of Pharmaceutics 111: 63-72, 1998).

In another example, a formulation comprises a peptidyl moiety conjugated to a hydrolysable polyethylene glycol (PEG) essentially as described by Tsubery et al., J. Biol. Chem. 279 (37) pp. 38118-38124. Alternatively, the formulation comprises a peptidyl, moiety conjugated to hydroxyethyl starch (HES) or polyglycine or serum protein moiety or serum protein-binding moiety. Without being bound by any theory or mode of action, such formulations provide for extended or longer half-life of the peptide moiety in circulation.

In another example, a formulation comprises a nanoparticle comprising the peptide moiety or other active ingredient bound to it or encapsulated within it. Without being bound by any theory or mode of action, delivery of a peptidyl composition from a nanoparticle may reduce renal clearance of the peptide(s).

In another example, a formulation comprises a liposome carrier or excipient to facilitate uptake into a cell. Liposomes are considered to interact with a cell by stable absorption, endocytosis, lipid transfer, and/or fusion (Egerdie et al., J. Urol. 142:390, 1989). For example, liposomes comprise molecular films, which fuse with cells and provide optimal conditions for wound healing (K. Reimer et al., Dermatology 195 (suppl. 2): 93, 1999). Generally, liposomes have low antigenicity and can be used to encapsulate and deliver components that cause undesirable immune responses in patients (Natsume et al., Jpn. J. Cancer Res. 91:363-367, 2000).

For example, anionic or neutral liposomes often possess excellent colloidal stability, since substantially no aggregation occurs between the carrier and the environment. Consequently their biodistribution is excellent, and their potential for irritation and cytotoxicity is low.

Alternatively, cationic liposomal systems, e.g. as described in Mauer et al., Molecular Membrane Biology, 16: 129-140, 1999 or Maeidan et al., BBA 1464: 251-261, 2000 are useful for delivering compounds into a cell. Such cationic systems provide high loading efficiencies. Moreover, PEGylated cationic liposomes show enhanced circulation times in vivo (Semple BBA 1510, 152-166, 2001).

Amphoteric liposomes are a recently described class of liposomes having an anionic or neutral charge at pH 7.4 and a cationic charge at pH 4. Examples of these liposomes are described, for example, in WO 02/066490, WO 02/066012 and WO 03/070735. Amphoteric liposomes have been found to have a good biodistribution and to be well tolerated in animals and they can encapsulate nucleic acid molecules with high efficiency.

USSN09/738,046 and U.S. Ser. No. 10/218,797 describe liposomes suitable for the delivery of peptides or proteins into a cell.

### Injectable Formulations

Injectable formulations comprising peptidyl or non-peptidyl compositions of the invention and a suitable carrier or excipient preferably have improved stability and/or rapid onset of action, and are for intravenous, subcutaneous, intradermal or intramuscular injection.

For parenteral administration, the peptidyl component or other active ingredient, may be administered as injectable doses of a solution or suspension in a physiologically acceptable diluent with a pharmaceutical carrier which can be a sterile liquid such as water or oil e.g., petroleum, animal, vegetable or synthetic oil including any one or more of peanut oil, soybean oil, mineral oil, etc. Surfactant and other pharmaceutically acceptable adjuvants or excipients may be included. In general, water, saline, aqueous dextrose or other related sugar solution, ethanol or glycol e.g., polyethylene glycol or propylene glycol, is a preferred carrier.

The injectable formulations may also contain a chelator e.g., EDTA, and/or a dissolution agent e.g., citric acid. Such components may assist rapid absorption of the active ingredient into the blood stream when administered by injection.

One or more solubilizing agents may be included in the formulation to promote dissolution in aqueous media. Suitable solubilizing agents include e.g., wetting agents such as polysorbates, glycerin, a poloxamer, non-ionic surfactant, ionic surfactant, food acid, food base e.g., sodium bicarbonate, or an alcohol. Buffer salts may also be included for pH control.

Stabilizers are used to inhibit or retard drug decomposition reactions in storage or in vivo which include, by way of example, oxidative reactions, hydrolysis and proteolysis. A number of stabilizers may be used e.g., protease inhibitors, polysaccharides such as cellulose and cellulose variants, and simple alcohols, such as glycerol; bacteriostatic agents such as phenol, m-cresol and methylparaben; isotonic agents, such as sodium chloride, glycerol, and glucose; lecithins, such as example natural lecithins (e.g. egg yolk lecithin or soya bean lecithin) and synthetic or semisynthetic lecithins (e.g. dimyristoylphosphatidylcholine, dipahnitoylphosphatidylcholine or distearoyl-phosphatidylcholine; phosphatidic acids; phosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelins. In one example, the stabilizer may be a combination of glycerol, bacteriostatic agents and isotonic agents.

In one example, the peptidyl or non-peptidyl component or other active ingredient of an injectable formulation is provided as a dry powder in a sterile vial or ampoule. This is mixed with a pharmaceutically acceptable carrier, excipient, and other components of the formulation shortly before or at the time of administration. Such an injectable formulation is produced by mixing components such as a carrier and/or excipient e.g., saline and/or glycerol and/or dissolution agent and/or chelator etc to form a solution to produce a “diluent”, and then and sterilizing the diluent e.g., by heat or filtration. The peptidyl component or other active agent is added separately to sterile water to form a solution, sterile-filtered, and a designated amount is placed into each of a number of separate sterile injection bottles. The peptide or other active agent solution is then lyophilized to form a powder and stored e.g., separately from the diluent to retain its stability. Prior to administration, the diluent is added to the injection bottle containing the dried peptidyl component or other active agent. After the predetermined amount of formulation is injected into the patient, the remaining solution may be stored, e.g., frozen or refrigerated.

In another example, the formulation is prepared as a frozen mixture ready for use upon thawing. For example, the peptidyl component or other active agent is combined with the diluent, sterile filtered into multi-use injection bottles or ampoules and frozen prior to use.

### Intranasal Formulations

For intranasal administration, powdery preparations having improved absorbability have been proposed. They are prepared e.g., by adsorbing physiologically active linear peptides onto a polyvalent metal compound such as hydroxyapatite or calcium carbonate (e.g., EP 0 681 833 A2). Peptides can be cyclized to improve their stability and resistance to peptidases in the nasal mucosa e.g., by synthesis as a continuous cyclotide or by oxidation of flanking cysteine residues. Alternatively, peptides may be stabilized in a particular conformation by means of artificially ‘stapling’ using chemical linkers e.g., Walensky et al., Science 305, 1466-1470 (2004).

Preferably, the peptide is dispersed homogeneously in and adsorbed homogeneously onto a physiologically acceptable particulate carrier, which can be a physiologically acceptable powdery or crystalline polyvalent metal carrier and/or organic carrier, whose mean particle size is in the range of 20 to 500 microns. In a preferred form, the RanBP2-type zinc finger domain according to any example hereof is formulated for intranasal delivery an alkyl-saccharide transmucosal delivery-enhancing excipient such as Intraveil (Aegis Therapeutics).

Suitable polyvalent metal component of the carrier include physiologically acceptable metal compounds having more than 2 valency, and may include, for example, zinc compounds. Such metal compounds are commonly used as excipients, stabilizers, filing agents, disintegrants, lubricants, adsorbents and coating agents for medical preparations. Zinc may be provided in the form of zinc chloride, zinc stearate or zinc sulfate.

Particulate organic carriers may be a fine powder from grain, preferably of rice, wheat, buck wheat, barley, soybean, corn, millet, foxtail millet and the like.

Such formulations may optionally comprise an absorption enhancer. Preferred absorption enhancers which may be one of the components of the nasally administrable composition is a pharmaceutically acceptable natural (e.g. cellulose, starch and their variants) or unnatural polymer material. A preferred embodiment of the cellulose and its variants is microcrystalline cellulose, methyl cellulose, ethyl cellulose, hydroxypropyl cellulose, hydroxypropylmethyl cellulose, hydroxypropylmethyl cellulose phthalate, cellulose acetate, cellulose acetate phthalate, carboxymethyl cellulose, low carboxymethyl cellulose sodium, carboxymethylethyl cellulose and the like. A preferable embodiment of the starch and its variants is corn starch, potato starch, rice starch, glutinous rice starch, wheat starch, pregelatinized starch, dextrin, sodium carboxymethyl starch, hydroxypropyl starch, pullulan and the like. Other natural polymers such as agar, sodium alginate, chitin, chitosan, egg yolk lecithin, gum arabic, tragacanth, gelatine, collagen, casein, albumin, fibrinogen, and fibrin may also be used as absorption enhancer. A preferable embodiment of the unnatural polymer is sodium polyacrylate, polyvinyl pyrrolidone, and the like. Preferred absorption enhancers are fine powder of rice, glutinous rice, starch, gelatine, dextrin, hydroxypropyl cellulose, hydroxypropylmethyl cellulose, polyvinyl pyrrolidone, egg yolk lecithin, gum arabic, tragacanth or a mixture thereof. More preferable absorption enhancers are fine powder of glutinous rice, starch, gelatine, hydroxypropyl cellulose, hydroxypropylmethyl cellulose, polyvinyl pyrrolidone, tragacanth or a mixture thereof. Even more preferable absorption enhancers are fine powder of glutinous rice or hydroxypropyl cellulose. Most preferable absorption enhancer is fine powder of glutinous rice. The mean particle size of the absorption enhancer is preferably not more than 250 microns, more preferably from 20 to 180 microns.

The above absorption enhancers may be used alone or in combination of two or more absorption enhancers in the physiologically acceptable powdery or crystalline carrier.

Water-soluble carriers are preferred to increase adsorption of the active substance in the nasal mucosa. Alternatively, this is achieved by homogeneous dispersion of the active substance in a water-insoluble carrier e.g., hydroxyapatite, calcium carbonate, calcium lactate, aluminum hydroxide or magnesium stearate, preferably in the presence of an absorption enhancer, and homogeneously adsorbing the active substance there onto.

Calcium carbonate, calcium lactate, aluminum hydroxide or magnesium stearate is usually used as a stabilizer, lubricant, agent to add lustre, excipient, dispersing agent or coating agent for a pharmaceutical preparation; however, it has been found that these compounds having a mean particle size of not more than 500 microns can be used as a carrier for the intranasal formulations, and promote absorption of a physiologically active substances into the body by nasal administration.

### Additional Components

In another example of the invention, a formulation comprises an additional component or compound e.g., an RNase molecule, protease inhibitor or RNase inhibitor.

### Modes of Administration

The present invention contemplates any mode of administration of a medicament or formulation as described herein, however one or a plurality of intranasal and/or injected and/or oral doses is preferred. Combinations of different administration routes are also encompassed e.g., intranasal and/or intravenous and/or oral.

Compositions according to the present invention are administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering the solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien Ed., Elsevier Publishers, New York, 1985; and in U.S. Pat. No. 4,778,810 (each incorporated herein by reference). Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface active agent, such as a nonionic surfactant (e.g., polysorbate-80), and one or more buffers. In some embodiments of the present invention, the nasal spray solution further comprises a propellant. The pH of the nasal spray solution is optionally between about pH 6.8 and 7.2, but when desired the pH is adjusted to optimize delivery of a charged macromolecular species (e.g., a therapeutic protein or peptide) in a substantially non-ionized state. The pharmaceutical solvents employed can also be a slightly acidic aqueous buffer (pH 4-6). Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5 micron mass median equivalent aerodynamic diameter (MMEAD), commonly about 1 micron MMEAD, and more typically about 2 micron MMEAD. Maximum particle size appropriate for deposition within the nasal passages is often about 10 micron MMEAD, commonly about 8 micron MMEAD, and more typically about 4 micron MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI) which rely on the patient's breath, upon pulmonary or nasal inhalation, to disperse the power into an aerosolized amount. Alternatively, the dry powder may be administered via air assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), starch e.g., hydroxyethyl starch (HES). Other suitable dry bulking powders include cellobiose, dextrans, maltotriose, pectin, sodium citrate, sodium ascorbate, and the like.

Standard methods are used to administer injectable formulations of the present invention.

### Medical Indications

The invention can be used for treatment or prophylaxis of any mammalian subject in need of, or already receiving, therapy for one or more consequences of aberrant gene expression associated with ssRNA or inappropriate ssRNA expression.

In one example, an composition of the present invention as described according to any example hereof is for treatment or therapy of a subject in need thereof e.g., for attenuation or alleviation or amelioration of aberrant gene expression associated with ssRNA or inappropriate ssRNA expression e.g., associated with disease such as cancer, neurodegenerative disease, cardiac myopathy, aberrant neovascularisation, or aberrant X-inactivation.

As used herein, the term “treatment” or “therapy” means to improve a subject's clinical state e.g., by reducing, alleviating, ameliorating or preventing one or more adverse indications of a disease, condition or syndrome. The treatment or therapy may involve complete abrogation of adverse indication(s) or comprise a partial improvement therein.

The subject will be a plant such as a crop plant or animal, such as a mammalian animal, e.g., a human or non-human animal, such as a domesticated non-human mammal, including a companion or laboratory mammal, e.g., selected from chimpanzees, monkeys, sheep, horses, cattle, goats, pigs, dogs, cats, rabbits, guinea pigs, hamsters, gerbils, rats and mice.

The present invention is described further in the following non-limiting examples:

### Example 1

### Materials and Methods

### 1.1 GenBank and Swiss-Prot Accession Codes

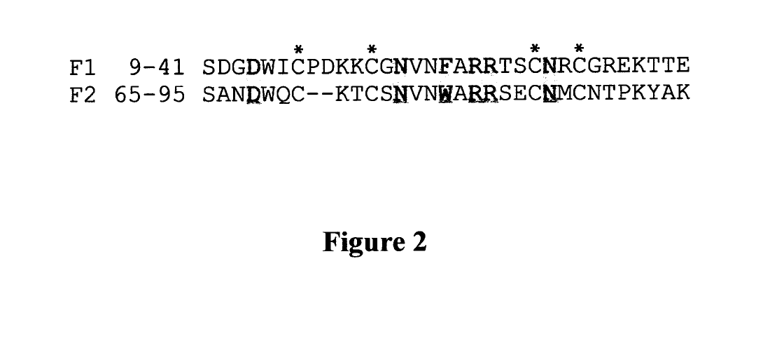

GenBank accession codes for ZRANB2 from various species as set forth in FIGS. 2, 11A and 21 include the following:

NM_{—}203350 (Homo sapiens); NM_{—}017381 (Mus musculus); NM_{—}031616 (Rattus norvegicus); NM_{—}001031297 (Gallus gallus); NM_{—}001090673 (Xenopus laevis); NM_{—}137848.2 (Drosophila melanogaster); NM_{—}062039 (Caenorhabditis elegans); CBG 19634 (Caenorhabditis briggsae); XM_{—}477574 (Oryza sativa); and NC_{—}001136 (Saccharomyces cerevisiae).

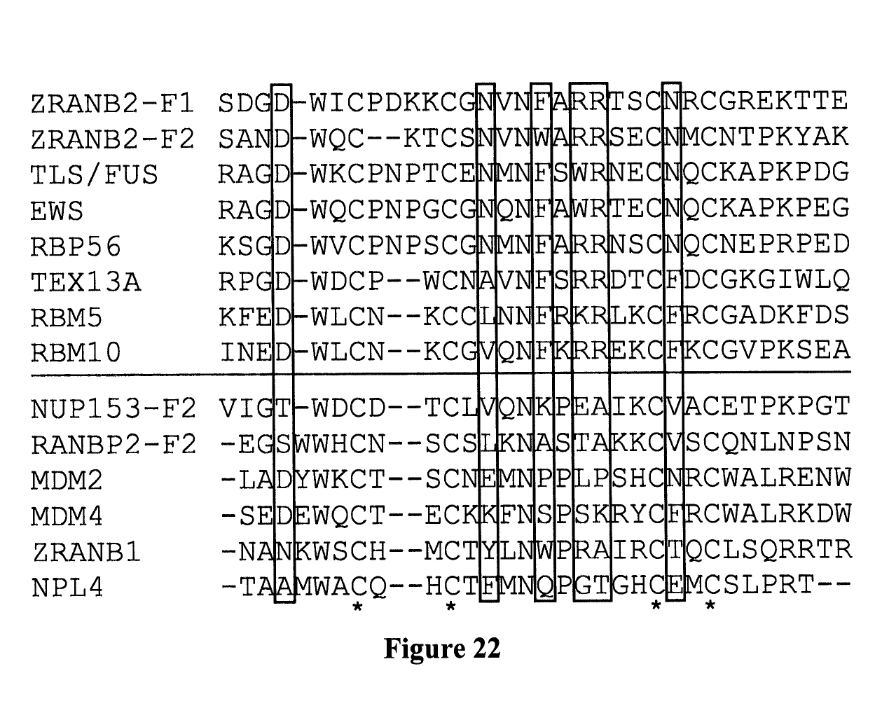

Swiss-Prot codes for ZRANB2 and related proteins as set forth in FIG. 22 are as follows:

ZRANB2 (O95218); TLS/FUS (P35637); EWS (Q01844); RBP56/TAFII68 (Q92804); TEX13A (Q9BXU3); RBM5 (P52756); and RBM10 (P98175); Nup153 (P49790); RanBP2 (P49792), MDM2 (Q00987); MDM4 (O15151); ZRANB1 (Q9UGI0); and Npl4 (Q8TAT6).

### 1.2 Sub Cloning, Expression and Purification

RanBP2-type ZnF domains from ZRANB2 and other human proteins (FIGS. 2, 11A, 21 and 22) were expressed as GST-fusion proteins and purified by glutathione affinity chromatography and either gel filtration or cation exchange chromatography as described in the following paragraphs.

Constructs comprising the F1 domain (residues 1-45 of ZRANB2), F2 domain (residues 65-95), or F1 and F2 domains i.e., F12 (residues 1-95 of ZRANB2) were created by PCR from the human Zranb2 gene, and point mutants e.g., (Figs. were constructed using overlap mutagenesis and expressed purified as previously described in Loughlin F. E. et al., 2008 Acta Crystallogr F 64: 1175-1177. Other constructs were expressed and purified similarly, with minor variations including the use of PreScission Protease (e.g., (Amersham Biotech) and cation exchange chromatography in the case of F12 according to established methods in the art. The folding of each purified protein was assessed by 1D ¹H NMR spectroscopy described below under section 1.6.

### 1.3 Systemic Evolution of Ligands by Exponential Enrichment (SELEX)

A ZRANB2-F12 SELEX protocol was developed, based on that used by Sakashita E. and Sakamoto H.1994 Acids Res 22:4082-4086. In brief, a library of ssRNA sequences was incubated with GST-F12 on glutathione Sepharose beads, and after washing protein—RNA complexes were eluted with glutathione and the selected RNA was reverse-transcribed and amplified by PCR. Sequencing of selected sequences was carried out after 7, 9, and 13 rounds of selection.

An oligonucleotide harbouring a 25-nucleoride (nt) random sequence surrounded by 2 primer binding sites with an estimated complexity of 1.2×10¹² (sampling complete sequence space for 20-nt sequences) was amplified by 10 rounds of PCR as previously described in Sakashita E., and Sakamoto H.1994 Nucleic Acids Res 22: 4082-4086, then purified by using a QIAquick gel extraction kit (e.g., QIAGEN) according to manufacturer's instructions. RNA was transcribed from 13 pmol of dsDNA template, then extracted with phenol/chloroform and ethanol-precipitated according to established methods in the art. The RNA pellet was resuspended in water (504), and unincorporated nucleotides were removed by Sephadex G-25 Quick Spin columns (Roche). RNA was quantified by absorbance at 260 nm.

Binding reactions were carried out in SELEX buffer [10 mM Mops (pH 7.0), 50 mM KCl, 5 mM MgCl₂, 5% glycerol, 1 mM DTT, 0.1% Triton, 0.1 M PMSF, Complete protease inhibitor]. Each 100 μL binding reaction contained 20-40 pmol of GST-ZRANB2-F12 immobilized on GSH beads (GE Healthcare), 1-5 μg of heparin sulfate, and 0.8-2.8 nmol of RNA and was gently mixed at 4° C. for 60 min. Unbound RNA was removed and the beads were washed 5 times with SELEX buffer (500 μL). GST-ZRANB2-F12 bound to RNA was eluted from the beads by incubating with 10 mM glutathione in 50 mM Tris-Cl, pH 8.0 (25° C., 15 min). The selected RNA was ethanol-precipitated and reverse-transcribed by using a complementary primer, then amplified by 10 or 18 rounds of PCR with Taq. The amplified pool of RNA was reapplied to a GSH column bearing fresh GST-F12 and the cycle was repeated. A total of 13 rounds of SELEX were completed. In rounds 5-13, Mops and NaCl concentrations were increased to 20 and 100 mM, respectively, in the selection buffer to increase selection stringency. After 7, 9, and 13 rounds of selection a fraction of the PCR products was digested with BamHI and subcloned into pUC119, and individual sequences were examined.

### 1.4 Gel Shifts

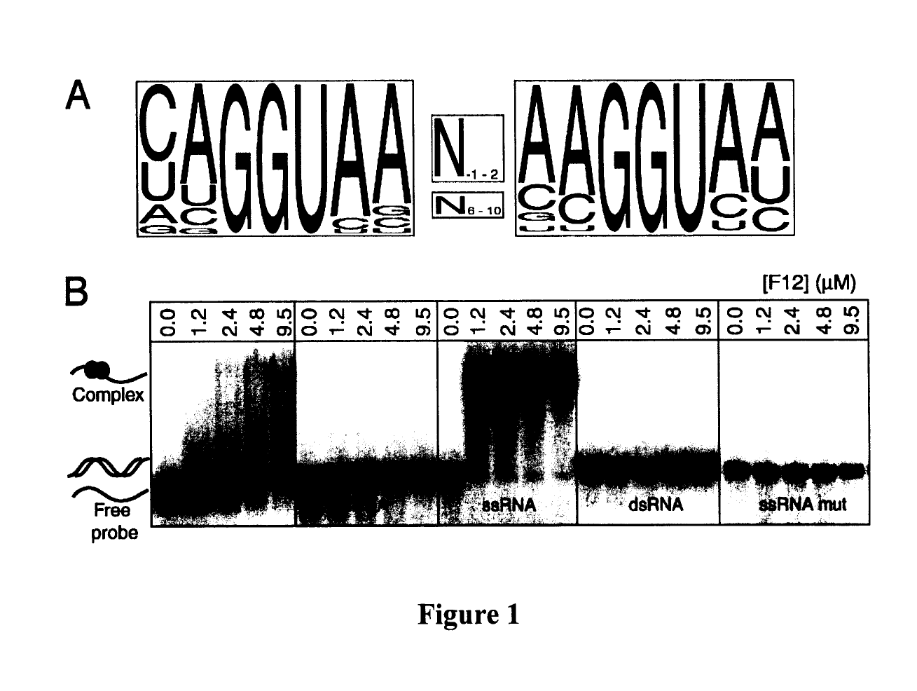

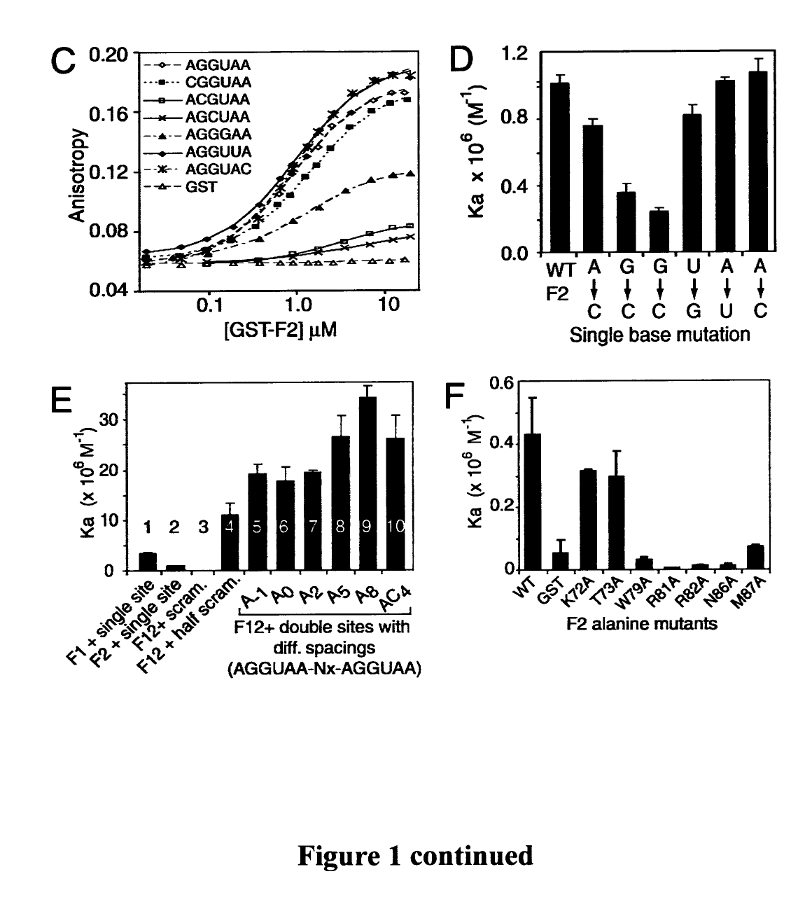

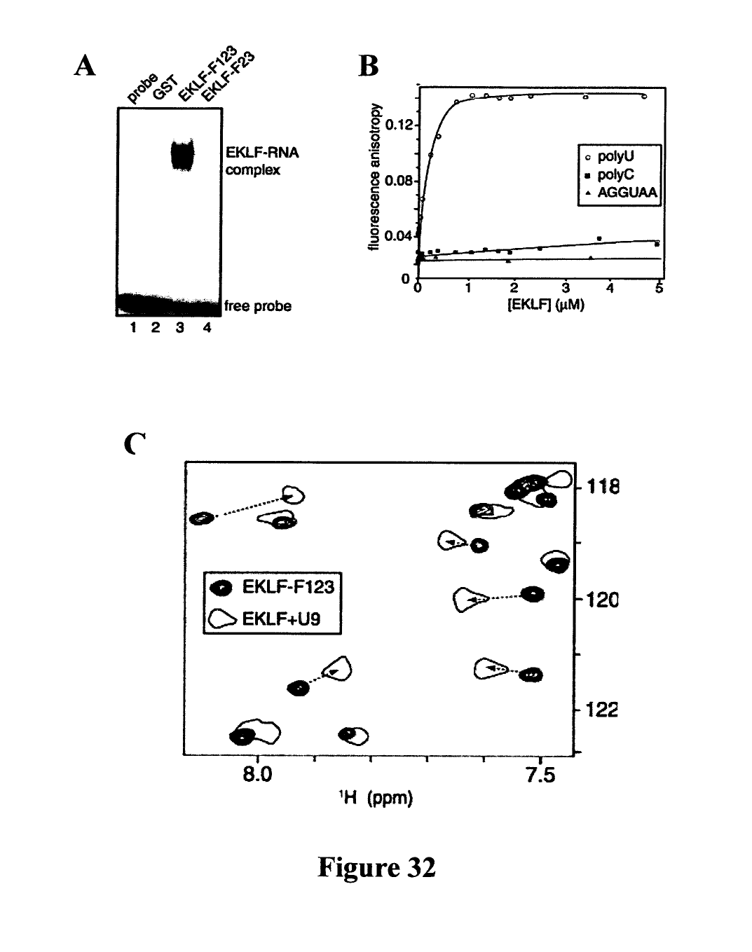

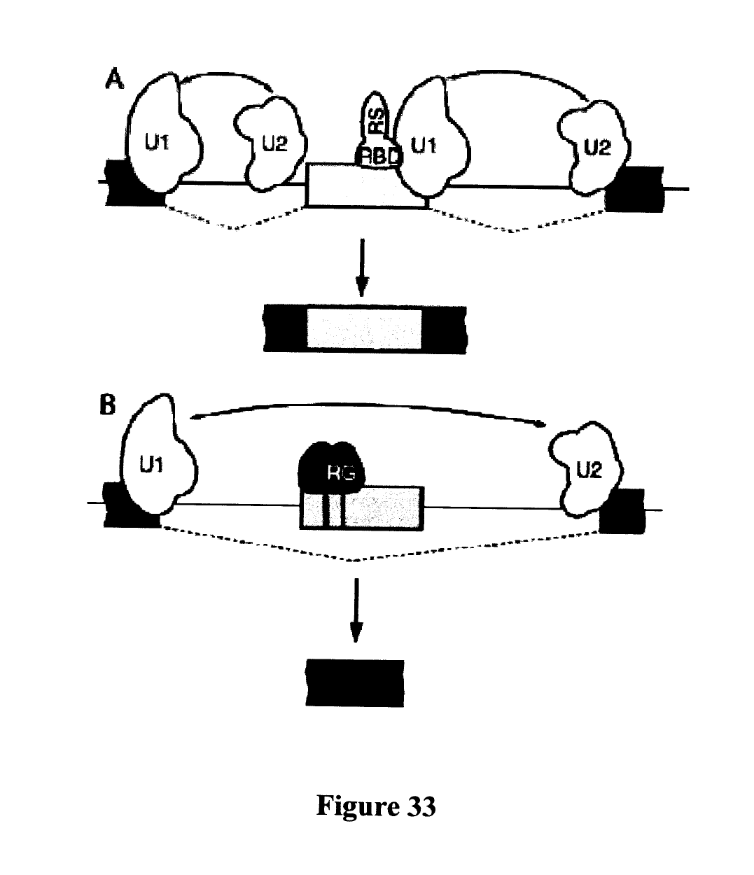

Gel Shift assays e.g., FIGS. 1B, 32A, were carried out using standard protocols. In brief, oligonucleotides were 5′ end-labelled with T4 polynucleotide kinase and annealed. Binding reactions were set up in a volume of 30 μL and contained a constant concentration of ³²P-labeled probe (0.1 pmol) and increasing concentrations of F12, in a buffer consisting of 10 mM Mops (pH 7.2), 50 mM KCl, 5 mM MgCl₂, 1 mM DTT, 0.03 mg/mL heparin, and 5% glycerol. Binding reactions were set up on ice and incubated at 4° C. for 30 min, after which time 15 μL of each sample was loaded onto a prerun 8% native polyacrylamide gel made up in 0.5× Tris-borate buffer, and electrophoresed (250V, 1 h, 4° C.). The oligonucleotide sequence used for gel shift experiments was GCAACCAGGUAAAGUCU (SEQ ID NO: 28); the site was mutated by changing the central GGU to CUG.

### 1.4 Fluorescence Anisotropy

Fluorescence Anisotropy titrations e.g., as shown in FIGS. 1D-F, 8 and 35, were carried out using standard protocols. In brief, 5′-Fluorescein-labeled RNA oligonucleotides were quantified by A₂₆₀, correcting for fluorescein absorbance by using A₄₉₃. For single-site experiments, the sequence GCAACCAGGUAAAGUCU (SEQ ID NO: 28), or single base mutants thereof, was used. For double-site experiments, two AGGUAA sites were separated by different length sequences as indicated, and the total oligonucleotide lengths were kept constant by the symmetrical addition of adenines to each end. The anisotropy titrations were performed with a starting RNA concentration of 50 nM in a buffer comprising either 10 mM Tris (pH 8.0), 50 mM KCl, 5 mM MgCl₂, 0.05 mg/mL heparin, and 1 mM DTT (for GST fusion constructs) or 10 mM Mops (pH 7.3), 50 mM NaCl, 0.05 mg/mL heparin, and 1 mM DTT (for ZRANB2-F12). RNasin (Promega) was also added to the protein solution to a concentration of 2 units/μL. Protein was titrated into this starting solution and anisotropy values measured on a Cary Eclipse fluorescence spectrophotometer fitted with manual polarizer and long-pass filters set to 475 and 515 nm for excitation and emission, respectively. Excitation and emission wavelengths were set at 495 and 520 nm, respectively (10-nm slits) and the temperature was maintained at 25° C. by using a block temperature controller. Association constants were determined by nonlinear least squares regression analysis using a 1:1 binding model.

### 1.5 Surface Plasmon Resonance

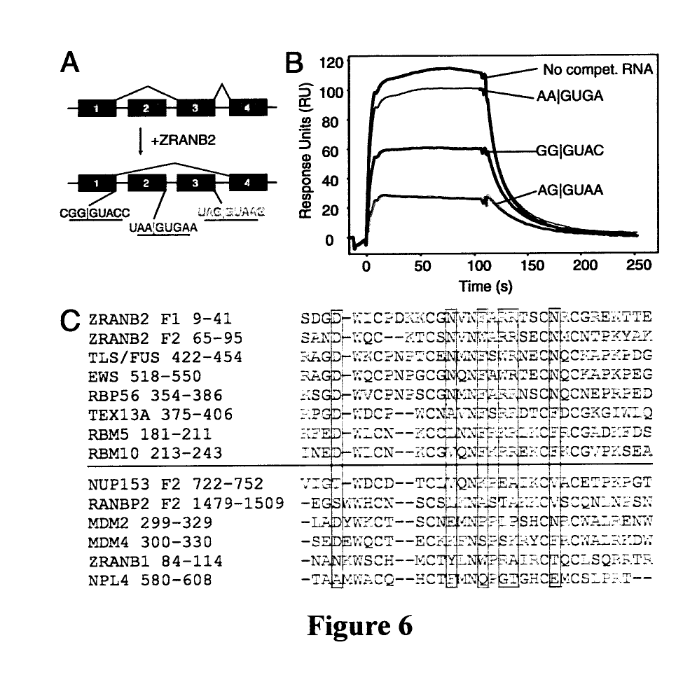

Surface plasmon resonance e.g., FIG. 6, was performed as described in the following paragraphs.

Competition binding experiments were carried out by flowing a solution of F12 over a streptavidin chip coated with a biotinylated ssRNA oligonucleotide (containing the sequence AGGUAA) in the presence of an unlabeled competitor oligonucleotide.

Binding experiments were performed in triplicate at 25° C. on a BIACORE 3000 (Biacore). A 5′ biotin-labeled RNA oligonucleotide containing the 5′ splice site from exon 3 of the Tra2β minigene (CUCUGAAUCUAGGUAAGAAAG; SEQ ID NO: 29) was purified by size exclusion chromatography (Superdex 75), and unlabeled competitor oligonucleotides i.e., GCGAGCGGUACGUAGA from exon 1 (SEQ ID NO: 30), AGGAAUAAGUGAAGCUG from exon 2 (SEQ ID NO: 31), and CUCUGAAUCUAGGUAAGAAAG from exon 3 (SEQ ID NO: 32) were used without further purification. Protein and RNA samples were dialysed into 10 mM sodium phosphate (pH 7.2), 50 mM NaCl, 1 mM DTT, and 0.005% NP20 and filtered. The biotinylated RNA was heated to 65° C. for 5 min, cooled on ice, and then immobilized onto a streptavidin-coated sensor chip to a total of approximately 160 relative units. Solutions of 750 nM F12 in the presence of 1 molar equivalent of each competitor RNA were injected in random order over the sensor chip (at a flow rate of 20 μL/min for 120 sec) and allowed to undergo a 120-sec dissociation time; a further 300-sec interval was allowed between injections. Data were analyzed by using BIA Evaluation software (Biacore).

### 1.6 X-Ray Crystallography

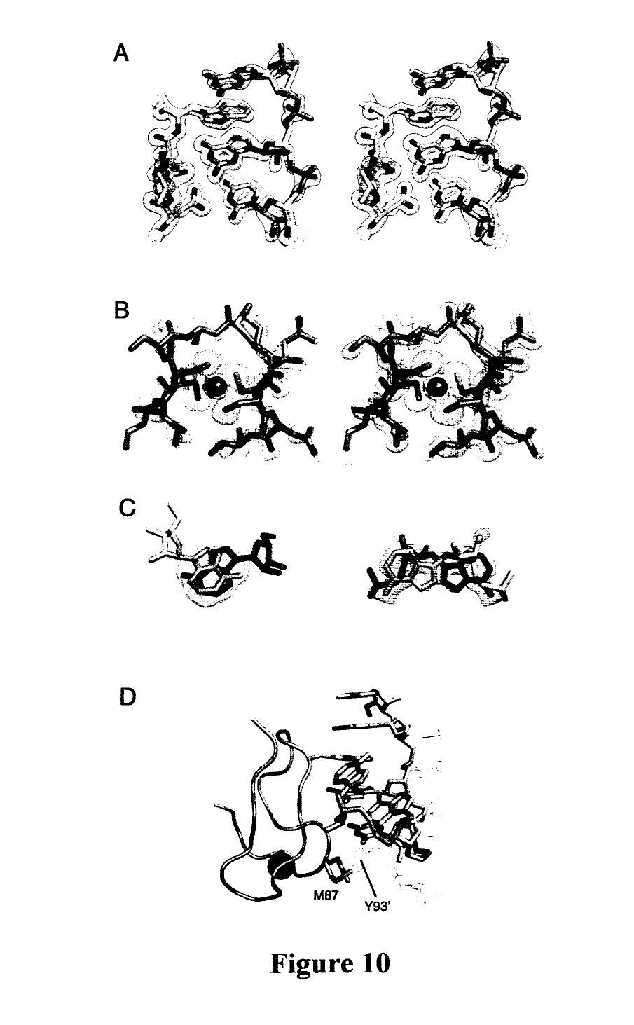

Crystallization and subsequent data collection of the ZRANB2-F2:RNA complex e.g., as shown in FIG. 10 hereof were preformed as described previously in Loughlin F. E. et al., 2008 Acta Crystallogr F 64: 1175-1177. The X-ray data were integrated and scaled with HKL2000 as previously described in Otwinowski Z., and Minor W. 1997, in Macromolecular Crystallography, Part A, eds Carter C W, Sween R M (Academic, New York), pp 307-326. The data quality was assessed with phenix.xtriage (as previously described in Zwart P. H. et al., 2005 CCP4 Newsletter Protein Crystallogr 43:7. Winder, Contribution 7), which suggested that all of the in-house data recorded with CuK_{α} radiation to 1.6-Å resolution could be usefully used for the SAD structure solution. Statistics for the data collection are summarized in Table 1 of Loughlin F. E. et al., 2008 Acta Crystallogr F 64: 1175-1177. The positions of the anomalous scatterers (Zn and 4 sulfur atoms of the coordinating cysteine residues) were located with SHELXD as described in Schneider T. R., and Sheldrick G. M., 2002 Acta Crystallogr D 58: 1772-1779. Phases were then calculated with either SHELXE (as previously described in Sheldrick G. M. 2002 Z Kristallogr 217: 644-650) or PHASER (as previously described in McCoy A. J. 2007 Acta Crystallogr D 63: 32-41) and improved by using density modification with DM as described in Cowtan K. D. and Main P. 1996 Acta Crystallogr D 52: 43-48.

Subsequent automated model building with ARP/wARP (e.g., as shown in FIG. 10 hereof) was performed as previously described by Langer G. et al., Nat. Prot. 3, 1171-1179, 2008. This yielded a nearly complete structure for the protein. The RNA, zinc ions, and solvent molecules were then added manually with COOT (as previously described Emsley P. and Cowtan K., 2004 Acta Crystallogr D 60: 2126-2132). Higher-resolution data to 1.4 Å were later collected on beamline 23-ID-D of GMCA-CAT at the Advance Photon Source (Argonne, IL). These data were again processed (as previously shown in table 1 of Loughlin F. E. et al., 2008 Acta Crystallogr F 64: 1175-1177) using HKL2000 (Otwinowski Z., and Minor W. 1997, in Macromolecular Crystallography, Part A, eds Carter C W, Sween R M (Academic, New York), pp 307-326) and used in the subsequent repeated rounds of refinement with REFMAC (11) interspersed with manual model fitting and checking with MOLPROBITY (as previously described in Davis I W et al., 2007 Nucleic Acids Res 35: W375-W383). The refinement converged to an R of 0.201 and Rfree of 0.235 with good stereochemistry.

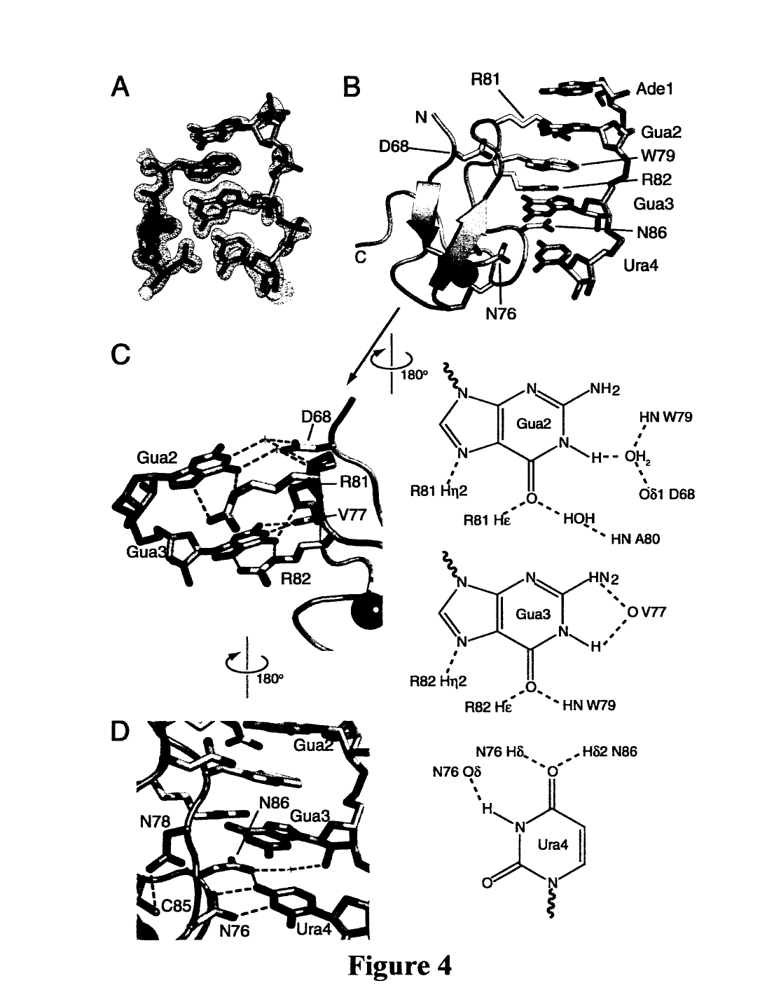

The final model contained disordered nucleotides at either end of the RNA chain. This disorder was required by the symmetry of space-group P6₅22 that places the same base from different molecules in the unit cell in overlapping positions. The possibility that the real symmetry of the crystal was in fact only P6₅ with the disordered bases of the 2 molecules in the asymmetric unit in different conformations was tested. Models with the protein alone and the protein with only the central 4 nt were refined in the lower symmetry space group. Attempts to break the resulting pseudo symmetry were made by placing the additional nucleotide in only 1 of the 2 molecules. All such attempts gave difference electron-density maps e.g., FIGS. 4 and 10 hereof, that clearly indicated overlapping disordered nucleotides and no significant reductions in either R or Rfree resulting from the additional parameters. It was therefore concluded that the structure was best modeled in the higher symmetry space group, P6522, with overlapping disordered bases for nucleotides 1 and 6.

### 1.6 NMR Spectroscopy

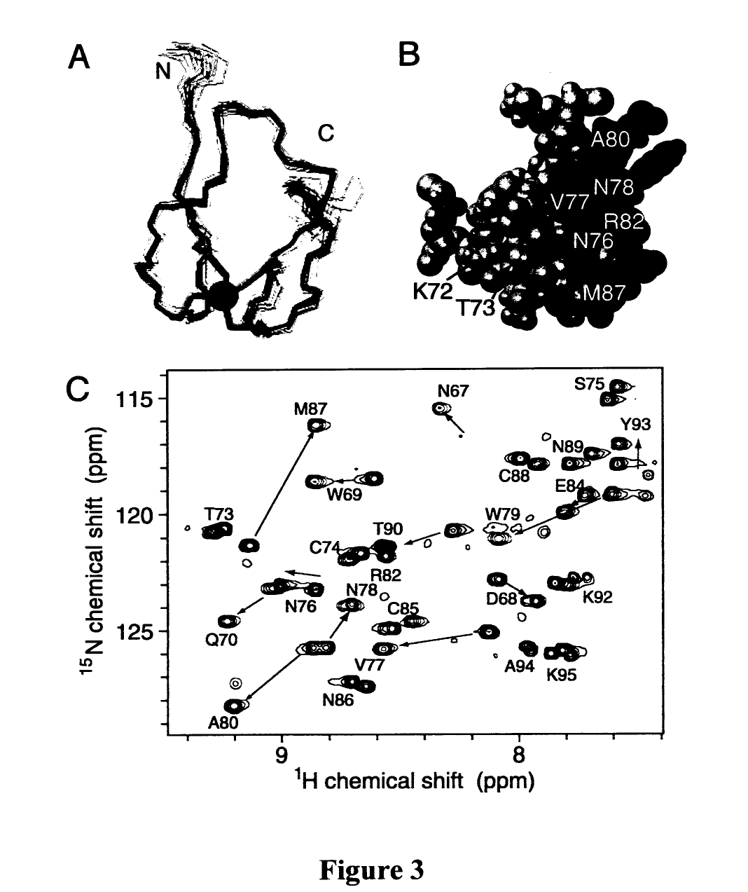

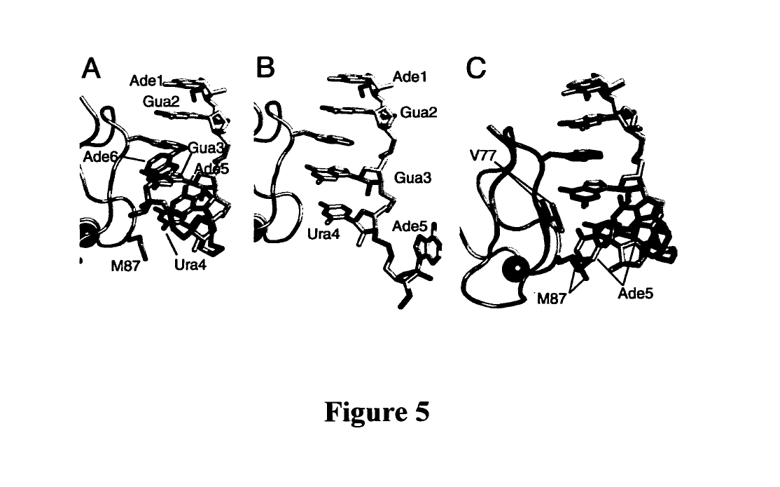

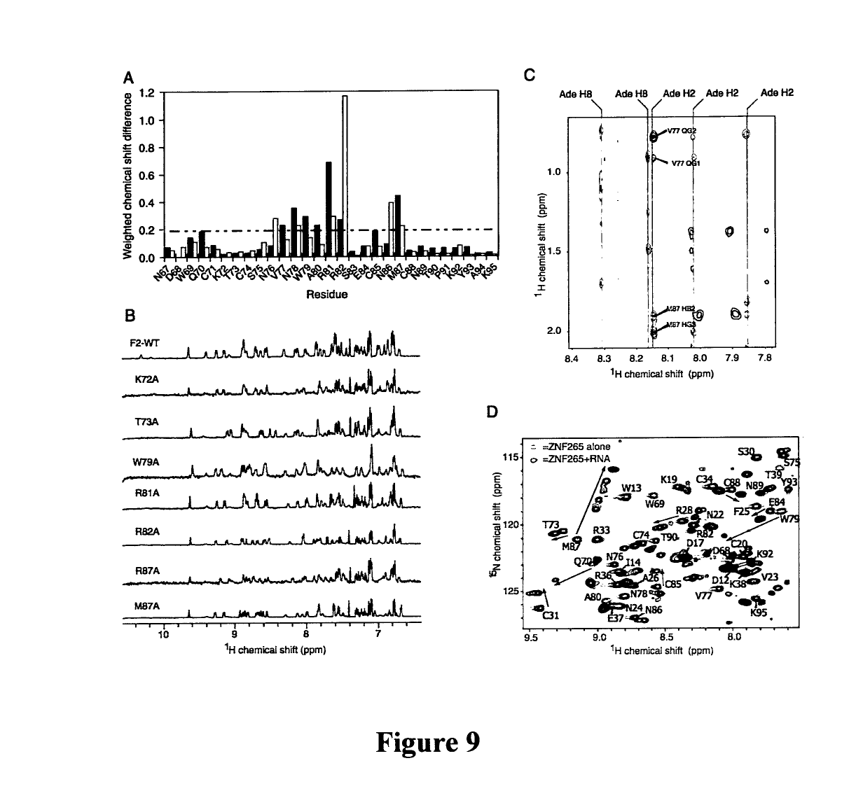

The structure of F2, RNA titrations, and full assignments for F12 and a F2:RNA complex were determined by using standard solution NMR experiments e.g., as shown in FIGS. 3, 9B and 11B. In brief, F2 and F12 were dialysed into 25 mM potassium phosphate (pH 7.0), 25 mM NaCl, and 0.5 mM DTT and concentrated to 0.4-1 mM. All spectra were recorded on a Bruker Avance 600 spectrometer equipped with a cryoprobe. 1H, 15N, and 13C assignments were obtained from HNCACB, CB-CA(CO)NH, HCCH-TOCSY, NOESY, and TOCSY spectra acquired at 2° C., 5° C., or 25° C. Dihedral angle restraints were obtained by analysis of an HNHA spectrum as previously described in Vuister G. W. and Bax A. 1993 J Am Chem Soc 115: 7772-7777. NMR data were processed by using XWINNMR/Topspin (Bruker) and analyzed with SPARKY (T. D. Goddard and D. G. Kneller, University of California, San Francisco). The structure of F2 was calculated from NOE and Ø angle constraints (e.g., FIGS. 5 and 9C) by using a protocol as described in Plambeck C. A., et al., 2003 J Biol Chem 278: 22805-22811.

For RNA titrations, deprotected RNA oligonucleotides i.e., having the sequence AGGUAA, CCAGGUAAAG (SEQ ID NO: 25), or AGGUAAAGGUAA (SEQ ID NO: 26) and the appropriate protein were dialyzed into 10 mM Mops (pH 7.2) and 0.5 mM DTT. Protein and RNA concentrations were calculated from A₂₈₀ and A₂₆₀, respectively. Final concentrations of complexes after titrations were typically approximately 0.1-0.5 mM. Assignments of the F2:RNA complex (1:1 or 1:1.2 molar ratio) were made at 25° C. as described above.

### 1.6 HADDOCK Docking

HADDOCK 2.0 (see Dominguez C., et al., 2003 J Am Chem Soc 125: 1731-1737, van Dijk A. D. and Bonvin A. M. 2006 Bioinformatics 22: 2340-2347, and van Dijk M., et al., 2006 Nucleic Acids Res 34: 3317-332) restrained molecular dynamics calculations were carried out by using the crystal structure of ZRANB2-F2:RNA. The following restraints were introduced: (i) ambiguous interaction restraints (2 Å) between all atoms of V77 and M87 and Ade5 as well between base specific atoms of Ade5 and corresponding atoms of V77 and M87, respectively; (ii) unambiguous restraints to fix the complex structure, with the exception of the side chains of V77 and M78 and the nucleotides Ade5 and Ade6; (iii) restraints based on the intermolecular NOEs between the Ade5 H2 proton and both the γ methyl groups of V77 and the Hβ and Hγ protons of M87; and (iv) dihedral restraints for Ade5 and Ade6 (C2′ endo, S-form) based on the presence of strong H1′-H2′ cross-peaks in TOCSY and COSY spectra of the complex. A total of 500 structures were calculated during 1 semiflexible, simulated annealing step without water-refinement. Only one distinct cluster of solution structures could be observed (cutoff of 3.5-Å rmsd based on the pairwise backbone rmsd matrix) indicating the presence of a single conformation consistent with all used restraints. The 10 best structures overlay with a rmsd (all atoms except Ade6) of 0.05 Å. HADDOCK runs were also performed with all restraints using Ade5 replace by Ade6 (assuming the observed NOEs are arising from the H2 proton of Ade6 rather than Ade5); however, no consistent structures were obtained.

### Example 2

### Identification of Zinc Fingers of ZRANB2 as Single-Stranded RNA-Binding Domains that Recognize 5′ Splice-Like Sequences

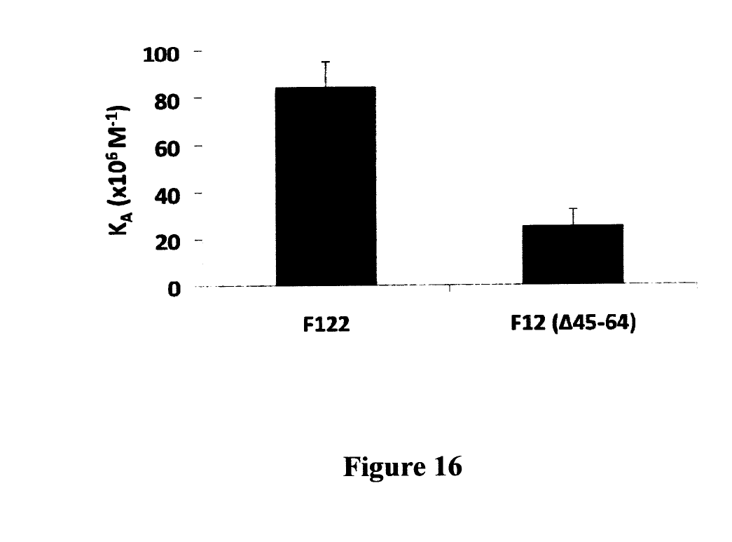

This example demonstrates that the double RanBP2 ZnF domain of ZRANB2 recognizes ssRNA, binding tandem copies of an AGGUAA motif with high affinity e.g., as shown inter alia in FIGS. 1 to 12 hereof. This example also investigates the structural basis for RNA recognition by ZRANB2 and demonstrates a large number of specific hydrogen bonds and base-stacked “ladders” involving tryptophan and 2 guanines e.g., as shown inter alia in FIGS. 16, 18, 24 and 30 hereof.

2.1 Identification of a High-Affinity ssRNA Ligand for ZRANB2-F12

This set of experiments examines whether or not the ZnFs of ZRANB2 can recognize ssRNA by in vitro site selection i.e., Systemic Evolution of Ligands by Exponential Enrichment (SELEX) assays as described in section 1.3 above.

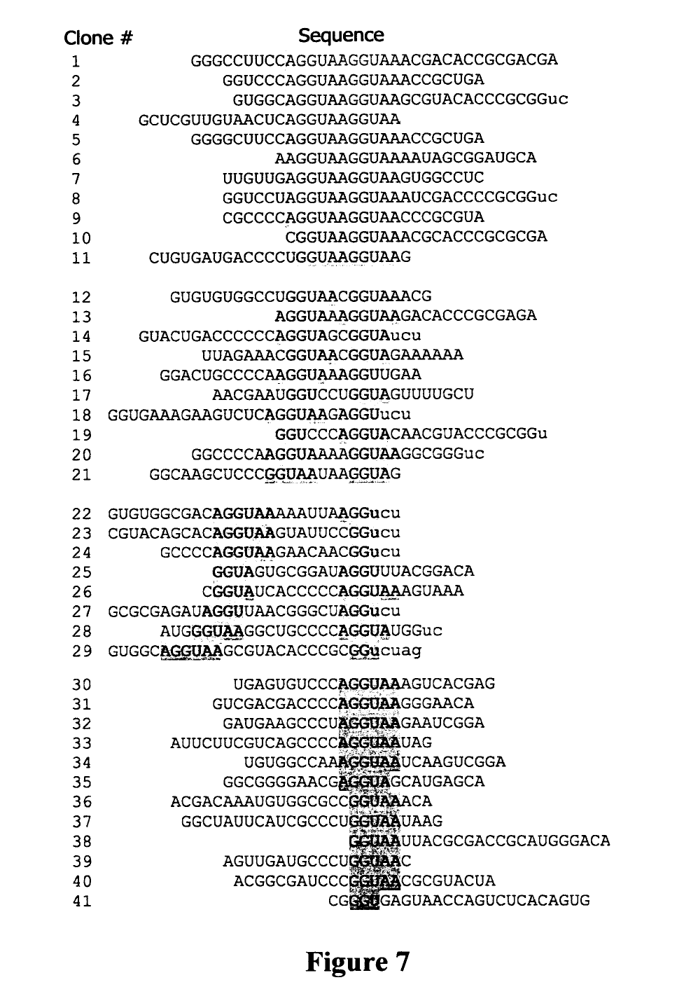

From a ssRNA pool containing a randomized 25-nt sequence, high-affinity RNA target sequences were selected by using a GST-fusion protein containing both ZnFs of human ZRANB2 (residues 1 to 95) Sepharose beads. After 7 rounds of selection, 26 unique clones were sequenced, all contained a GGUA or a AGGU motif, and 15 contained the longer AGGUAA motif. Further selection enriched the RNA pool in sequences containing multiple AGGUA motifs, such that after 9 rounds of selection 15 of 33 unique clones contained 2 GGUA motifs. In 11 of these 15 clones, the 2 motifs occurred either in tandem or separated by 1 nt. Alignment of these sequences as shown in FIG. 7 hereof. Each of the zinc finger domains F1 and F2 of ZRANB2 recognized an AGGUAA site (FIGS. 1A and 7). MFOLD (Zuker M. 2003 Nucleic Acids Res 31: 3406-3415) did not reveal any consistent secondary structure predictions for these sequences, suggesting that the binding is sequence-driven rather than structure-driven.

To determine whether the AGGUAA motif was sufficient for binding, the binding of the double finger construct F12 to a 17-nt RNA oligonucleotide containing a single AGGUAA motif was tested as described in Section 1.4 above with dsDNA, ssDNA, and dsRNA also tested as controls. Gel shift results are shown in FIG. 1B. As shown in FIG. 1B, the interaction was strongly selective for ssRNA, consistent with a role for ZRANB2 in mRNA processing. Mutation of the central GGU, which is the most highly-conserved element in the SELEX consensus to CUG, abrogated the interaction, showing that the interaction is sequence specific (FIG. 1B).

### 2.2 Sequence Specificity of the RNA-ZRANB2 Interaction

This set of experiments investigates whether or not the repetition of the AGGUAA motif in the SELEX sequence alignment means that each of the ZnFs can recognize a single site.

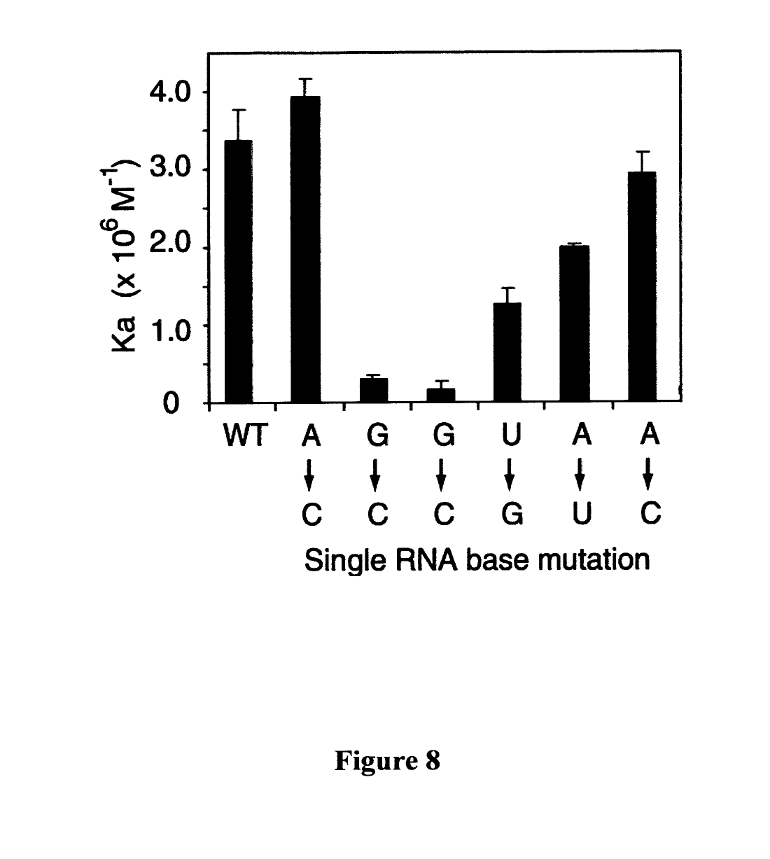

Constructs of the two individual domains, F1 (FIG. 2, residues 1-45) and F2 (FIG. 2, residues 65-95), were cloned expressed and purified as GST fusions described in section 1.2 above, and tested their ability to bind ssRNA by fluorescence anisotropy as described in section 1.5 above, using a ssRNA oligonucleotide bearing a 5′ fluorescein tag and containing a single AGGUAA site. F1 and F2 bound to this sequence with association constants of 3×10⁶ M⁻¹ and 1×10⁶ M⁻¹, respectively (FIGS. 1C and 1D and FIG. 8), demonstrating that both F1 and F2 can bind one AGGUAA site. The data also demonstrated that the central GG sequence was most important for binding by measuring the affinity of each finger for a series of ssRNA oligonucleotides that each contained a single purine 7 pyrimidine mutation in the AGGUAA motif (FIG. 1D and FIG. 8).

The binding of F12 to ssRNA containing two AGGUAA sites was then measured and the results are shown in FIG. 1E. The affinity of F12 for the sequence AGGUAAAGGUAA (SEQ ID NO: 26) was 1.9×10⁷ M⁻¹ (FIG. 1E, lane 6). Randomizing one of the AGGUAA motifs (to give the sequence AGAAUGAGGUAA set forth in SEQ ID NO: 26; FIG. 1E, lane 4) reduced the binding by a small, but reproducible, amount, although it was notable that binding was still significantly stronger than that of a single finger to a similar sequence (FIG. 1E, lanes 1 and 2), indicating that the additional finger in the F12 construct could still make contact with the scrambled second site. Scrambling both sites effectively eliminated binding (FIG. 1E, lane 3).

The importance of the spacing between AGGUAA motifs was assessed by measuring binding of F12 to double sites that were separated by −1 to 8 nt (where −1 denotes the sequence AGGUAAGGUAA SEQ ID NO: 33; FIG. 1E, lanes 5-10). Surprisingly, the affinity of the interaction increased monotonically as an increasing number of adenines were inserted between the sites. Further, a double site containing a cytosine-rich spacer (FIG. 1E, lane 10) had the same affinity for F12 as a penta-adenine spacer (FIG. 1E, lane 8), demonstrating that the identity of the intervening bases is unimportant.

These data demonstrate that the double ZnF domain of ZRANB2 recognizes ssRNA carrying the consensus sequence AGGUAA(N₁₋₈)AGGUAA (SEQ ID NOs: 34-41). Strikingly, AGGUAA is almost identical to the conserved consensus sequence for the 5′ splice site across metazoans (described previously in Ast G. 2004 Nat Rev Genet. 5: 773-782; Zhang M. Q. 1998 Hum Mol Genet. 7: 919-932) and resembles the 3′ splice site consensus (CAGG).

### 2.3 Solution Analysis of the ZRANB2:RNA Interaction

This set of experiments investigate the structural interaction of ZRANB2:RNA.

To provide structural insight into the ZRANB2:RNA interaction the inventors first determined the structure of F2 by using NMR spectroscopy as described in section 1.8 above, with results shown in FIG. 3 and FIG. 9B and FIG. 11B. The structure is well defined and comprises 2 Short β-hairpins sandwiching a zinc ion that is ligated by the 4 conserved cysteines. The fold is consistent with that of F1 (Plambeck C. A., et al., 2003 J Biol Chem 278: 22805-22811) and other structures from this class of ZnFs, including domains from HDM2 (Yu G. W., et al., 2006 Protein Sci 15: 384-389), Npl4 (Wang B, et al., 2003 J Biol Chem 278: 20225-20234), and Nup153 (Higa M. M., et al., 2007 J Biol Chem 282: 17090-17100).

The inventors then carried out chemical-shift perturbation experiments, titrating short (6 or 10 nt) RNA oligonucleotides containing a single AGGUAA motif into ¹⁵N-labeled F2. Significant chemical exchange broadening was observed for a subset of signals during the titration, but all signals reappeared after the addition of 1 molar equivalent of RNA (FIG. 3C), and no further changes were observed if more RNA was added, consistent with the formation of a well-defined 1:1 complex.

Mapping the most significant chemical-shift changes onto the structure of F2 (FIG. 3B and FIG. 9A) revealed a single contiguous surface. The binding surface comprises a mixture of amino acid types, including aromatic (W79), aliphatic (V77, A80, M87), polar (N76, N78, N86), and charged (R81, R82) residues. These residues were almost completely conserved in F1, suggesting that each ZnF recognizes RNA in the same manner.

Corroboration of the structural data was carried out by examining the effect of alanine point mutations on the RNA-binding ability of F2 with results shown in FIG. 1F. The data demonstrates that alanine substitutions of W79, R81, R82, N86, and M87 significantly reduced the association constant (the correct folding of each mutant was confirmed by NMR; FIG. 9B). In contrast, much smaller changes in affinity were observed for K72A and T73A, which are oriented away from the RNA contact surface.

¹⁵N-HSQC titrations were also carried out by using the double finger construct (F12; residues 1-95) and the double site oligonucleotide. AGGUAAAGGUAA (SEQ ID NO: 26). Chemical shifts for the free F12 protein were essentially unchanged from those in the 2 individual finger constructs, and no signals were observed in the ¹⁵N-HSQC of F12 for residues in the 25-residue linker. This titration (FIG. 9D) gave rise to the same pattern of chemical-shift changes as the two single-finger experiments. Only 3-4 new signals appeared, most likely from linker residues. However, most of the linker residues remained unobservable, indicating that the linker does not become ordered upon RNA binding.

2.4 Structural Basis for ssRNA Recognition by ZRANB2

These set of experiments demonstrate the that the interaction between the ssRNA and ZRANB2 is mediated predominantly by hydrogen bonds between protein side chains and the bases, together with a tryptophan stacking motif.

Only a small number (<10) of intermolecular NOEs were observed between F2 and RNA; intermediate chemical exchange broadened signals from key residues at the protein: RNA interface. The inventors therefore crystallized F2 in complex with a 6-nt RNA with the sequence AGGUAA and determined the structure of the complex to a resolution of 1.4 Å by X-ray crystallography analysis as described under section 1.7 above. The overall quality of the structure, as judged by Molprobity (Davis I. W., et 2007 Nucleic Acids Res 35: W375-383), was excellent. All F2 residues lie in the favored region of the Ramachandran plot, and Molprobity scores the structure in the top 1% of all structures determined to comparable resolution. All RNA nucleotides had acceptable sugar puckers and residues Ade1-Ura4 have acceptable backbone conformations.

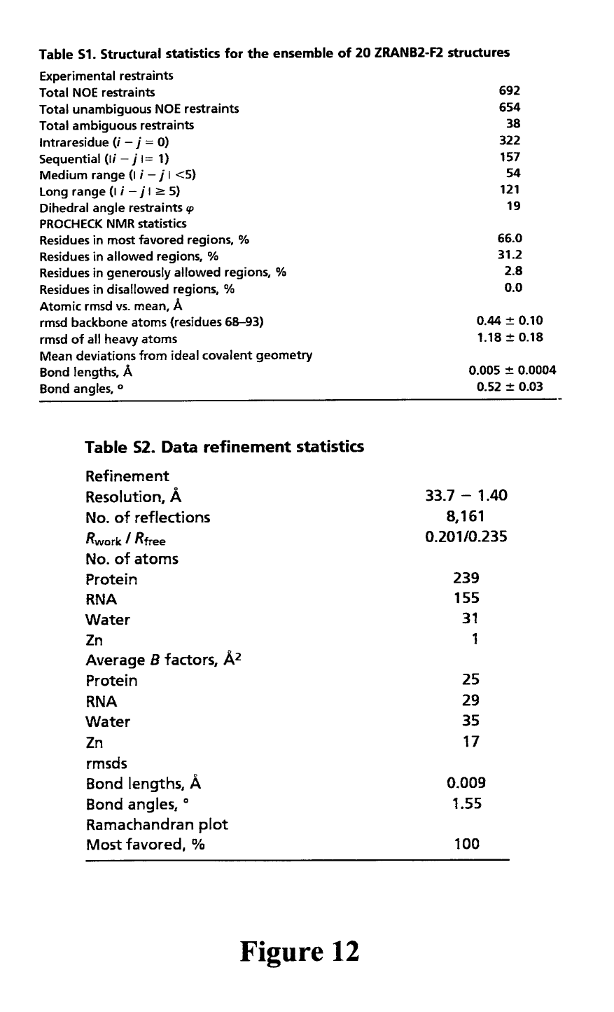

In the structure (FIG. 4, FIG. 12B, and FIG. 10), F2 adopted the same backbone fold as it did free in solution. Electron density for Gua2, Gua3, and Ura4 was unambiguous and clearly revealed the mode of recognition of these bases (FIG. 4). Most prominently, a tryptophan side chain (W79) stacked between Gua2 and Gua3; the plane of the indole side chain was parallel with those of the 2 purines and made extensive contacts with both bases (FIGS. 4A and B). Although this purine-Trp-purine ladder appeared well-suited to direct recognition of single-stranded nucleic acids, a motif of this type has not been previously observed in any protein-nucleic acid structure to the inventors' knowledge.

Gua2, Gua3, and Ura4 made a number of hydrogen bonds to side chains and the backbone of the protein (FIGS. 4C and D). A striking feature was the bidentate interaction of both Gua2 and Gua3 with an arginine side chain. Gua2 formed 2 hydrogen bonds to R81 side chain protons: the carbonyl oxygen O6 with H^{ε} and one of the H^{η} protons with N7. O6 also formed a water-mediated hydrogen bond with the backbone amide proton of R81. The imino proton on the Watson-Crick face of Gua2 formed a second water-mediated hydrogen bond, whereby the water interacted with both the D68 carboxylate group and the A80 backbone amide. Gua3 formed a bidentate interaction with R82, and again the O6 formed a second hydrogen bond to a backbone amide, in this case W79. The backbone carbonyl of V77 formed hydrogen bonds with both the imino proton and the amino group of Gua3, and a water-mediated hydrogen bond connected the 2′ hydroxyl group of Gua3 with the side chain of N86. Ura4 formed 3 side-chain-mediated hydrogen bonds to F2. The O4 carbonyl group recognized side-chain amide protons in both N76 and N86, and the side-chain carbonyl group of N76 makes a hydrogen bond with the Ura4 imino proton.

These interactions explain the strong observed preference for a core GGU sequence. The pattern of hydrogen bond donors and acceptors observed for the interaction with each guanine (FIG. 4C) was not compatible with either adenine or cytosine, and although uridine had a similar polarity to guanine (O4 replaced O6 and N3 replaced N1 on the Watson-Crick face) it would have been unable to form the additional hydrogen bond made to each guanine N7. Recognition of Ura4 relied on a hydrogen-bond network involving N76, N78, C85, and N86 (FIG. 4D), which orients N76 such that the polarity of its side chain was complementary to uridine.

### 2.5 Conformation of Ade1, Ade5, Ade6

This set of experiments demonstrates the likely conformation of the three adenines, Ade1, Ade5 and Ade6. Although electron density for the GGU was well-defined and unambiguous, refinement of the adenines was more difficult. Ade1 was modeled at 50% occupancy into a conformation in which the base extends the Gua2-W79-Gua3-Ura4 stack (FIG. 4), but this nucleotide did not directly contact F2. Ade5 was modeled into 2 different conformations (FIGS. 5A and B), and for one of these, density could additionally be observed for Ade6 (FIG. 5A). In this latter conformer, the RNA backbone changed direction by about 90° and folded back on itself. Ade5 made contacts with the backbone and ribose ring of Ura4 and the Ade6 base was stacked coplanar with Ade5. In the alternate conformer, Ade5 was oriented away from the protein (e.g., FIG. 5B).

Chemical-shift perturbation analysis e.g., as shown in FIGS. 3, 9 and 32 were performed. These studies revealed inter alia that M87 underwent one of the largest chemical-shift changes upon RNA binding and the alanine point mutation M87A reduced the RNA binding affinity of F2 (FIG. 1F). However, no contacts were observed between this residue and the RNA in the crystal structure. Further, the few intermolecular NOEs that could be assign with confidence were between the H2 proton of a single adenine and the side chains of M87 and V77 (FIG. 9C). Neither of the conformations in the crystal was consistent with these NOEs, suggesting that either the Ade5-Ade6 dinucleotide underwent significant motion in solution or the conformation differed in this region in solution. Examination of packing in the crystal (FIG. 10D) showed that a tyrosine side chain from a symmetry-related molecule was next to M87, and it was possible that crystal-packing forces disturbed Ade5 and Ade6 from their preferred solution positions.

To assess the likely conformation of Ade5 and Ade6 a restrained molecular dynamics calculation was carried out using HADDOCK as described above in section 1.9 (Dominguez C. et al., 2003 J Am. Chem. Soc. 125: 1731-1737). In this calculation, the positions of the AGGU sequence and the entire protein other than M87 and V77 were fixed. Ade5 and Ade6 were allowed to reorient freely under the influence of the force field and the NOEs to the protein. When the NOEs were directed to Ade5 H2, the calculations revealed a single conformer that was consistent with all of the NOEs (FIG. 5C). In this structure, Ade5 was in a position similar to that exhibited in the X-ray conformer shown in FIG. 5A (but translated approximately 5 Å toward the protein), suggesting that Ade5 and Ade6 spent at least a proportion of their time in such a conformation.

### 2.6 Binding Preferences of ZRANB2 Zinc Finger Domains Corroborate Functional Splicing Data

This set of experiments investigates functional role for ZRANB2 and provides supporting data for a role for ZRANB2 in alternative splicing e.g., as shown in FIGS. 6A-6C hereof.

ZRANB2 can alter the splicing of Tra2-β, GLUR-B, and SMN2 reporter genes in splicing assays. ZRANB2 promotes the exclusion of exon 3 from a Tra2-β reporter gene containing 4 exons (FIG. 6A). Given the SELEX data provided herein it was notable that the sequence of the 5′ splice site of exon 3 is AG/GUAA, whereas those of exons 1 and 2 were GG/GUAC and AA/GUGA, respectively/(where the slash represents the cleavage site in the splicing reaction).

To test whether the tandem zinc finger domain of ZRANB2 would bind preferentially to the 5′ splice site of exon 3, surface plasmon resonance competition experiments were carried out. A 5′-biotinylated oligonucleotide containing the sequence AGGUAA was immobilized on a streptavidin-coated Biacore chip and a solution of ZRANB2-F12 containing unlabeled oligonucleotides corresponding to the 5′ splice sites of exons 1, 2, or 3 was injected and results are shown in FIG. 6B. As shown in FIG. 6B, the oligonucleotide from the 5′ splice site of exon 3 caused the largest reduction in binding of ZRANB2 to the chip, indicating that the protein had a clear preference for this sequence above the others.

The functional data obtained for ZRANB2 herein point strongly toward a role for ZRANB2 in alternative splicing. The binding of ZRANB2 to both U170K and U2AF35 indicated that it acted early in the splicing reaction, consistent with a role in splice site choice. The RNA-binding properties of ZRANB2 draw parallels with canonical SR proteins such as ASF/SF2 and SC35, although unlike these latter proteins ZRANB2 does not localize to nuclear speckles and no clear consensus sequence could be found with canonical SR proteins. This contrasts sharply with the well defined consensus sequence obtained here and indicates a role for ZRANB2 in regulating specific transcripts, rather than a global role in constitutive splicing.

The target sequence for a single ZRANB2 ZnF strongly resembles the 5′ splice site, which is conserved across all metazoans. It is therefore possible that ZRANB2 acts by binding directly to a subset of 5′ splice sites so as to prevent recognition of those sites by the spliceosome. Such a mode of action is supported by the affinities of ZRANB2 for the different splice sites in the Tra2-0 minigene. Indeed in each of the exons excluded from the transcripts of the GluR-B, SMN2 and Tra2-β minigenes after the addition of ZRANB2, a single or double (A)GGUA(A) site is present at or around the 5′ splice site of the major excluded exon. Given that 3′ splice sites also display a GG dinucleotide, this indicates that the two ZRANB2 ZnFs might simultaneously contact both splice donor and splice acceptor sites within the same transcript to influence splicing.

Alternatively, ZRANB2 may recognize cryptic splice sites containing 1 or 2 AGGUAA sequences, either activating or suppressing their use, similar to that of the Drosophila protein PSI and the pseudo 5′ splice site in the P-element transportase pre-mRNA. The fact that ZRANB2 can accommodate a range of spacings between 2 AGGUAA motifs indicates that the protein might recognize clusters of these motifs rather than a strict tandem site.

### 2.7 Conservation of the ZRANB2-F12:RNA Interaction.

This set of experiments demonstrates conservation of ZRANB2 among species implicates conservation of ZnF: RNA interactions among species e.g., as shown in FIGS. 2, 11A and 21 hereof.

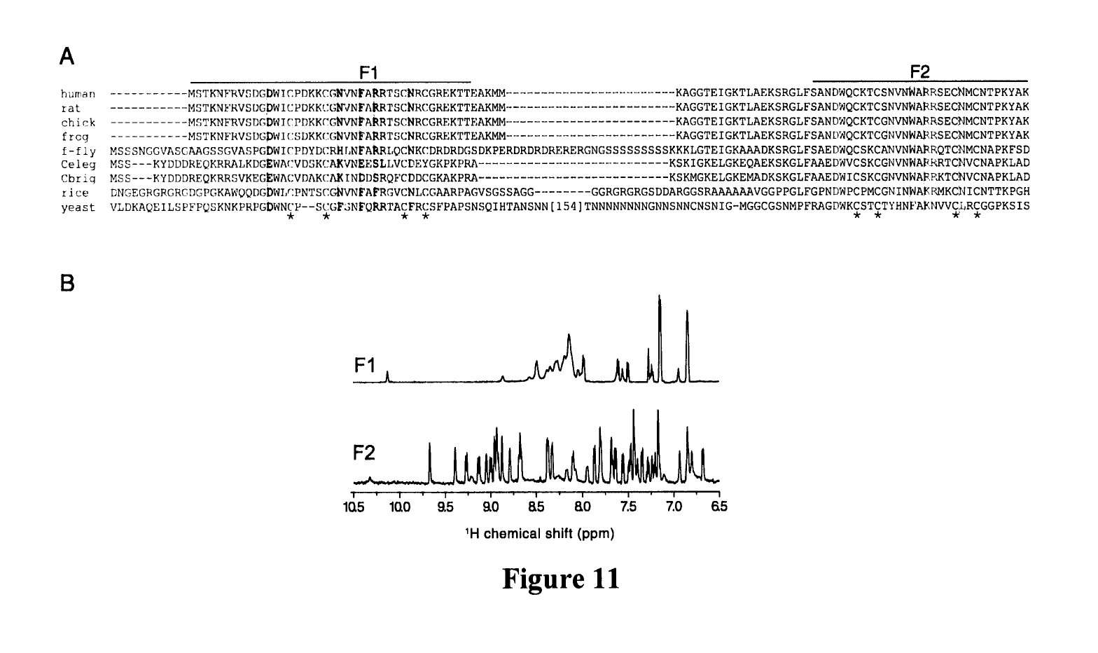

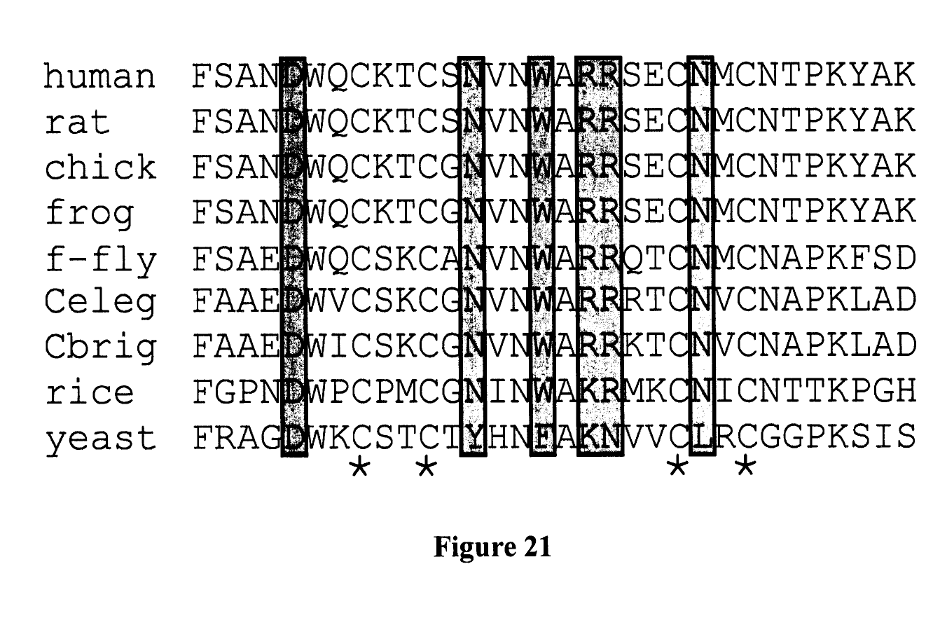

Sequence alignment of ZRANB2 zinc finger domains from 8 different species including human, rat, chick, frog, rice yeast is show in FIGS. 2, 11A and 21. The results demonstrate that the ZnFs of ZRANB2 were highly conserved from Xenopus to humans, and a number of the residues that are important for RNA recognition were conserved in insects and nematodes. It is notable that the N-terminal finger of the Caenorhabditis elegans protein is missing one of the conserved zinc-binding cysteines and was disordered in solution (FIGS. 11A and 21). Yeast and rice also contained orthologs of ZRANB2 in which the RNA recognition surface of the ZnFs was partly conserved (FIGS. 2, 11A and 21).

Further, examination of the F2:RNA structure revealed that some amino acid substitutions most likely retain specificity for GGU. For example, the asparagine that replaces R82 in the yeast protein could still hydrogen-bond, via its side-chain NH2 moiety, to the O6 of Gua3. This high degree of conservation observed suggests that the RNA binding activity and sequence specificity of ZRANB2 is part of an ancient and important function.

2.8 A family of RanBP2 ZnF ssRNA-Binding Domains

Alignment of protein sequences obtained from a BLAST search reveals that subsets of the residues in ZRANB2-F2 domain that directly contact the GGU motif were also present in several other human RanBP2 ZnFs (e.g., FIG. 22). RBP56 shares all of these residues and accordingly the inventors reasoned that RBP56 should exhibit the same sequence specificity and mode of recognition as ZRANB2. Both TLS and EWS carry a single change: R81 to W. Inspection of the F2:RNA structure revealed that the indole H^{N} of this tryptophan could still make a hydrogen bond with the O6 carbonyl of Gua2, thereby mimicking the base specificity imparted by the arginine. Tex13a, RBM5, and RBM10 all had changes to the 2 residues that specify uridine at position 4 (N76 to A/L/V and N86 to F). Notably, the N76L and N86F changes in RBM5 gave rise to a surface that was similar in overall shape but lacked hydrogen-bonding capacity. It is therefore possible that RBM5 accepts either pyrimidine in this position. Other proteins, including MDM2 (a regulator of p53), contain RanBP2 ZnFs that display 1 or 2 of the RNA-binding residues that were identified in this study. The inventors reasoned that if such domains can bind RNA, their sequence specificity will likely be very different. In contrast, the RanBP2 ZnF in Npl4 contains none of the RNA-binding residues and instead harbors a conserved Thr-Phe dipeptide that mediates an interaction with ubiquitin. Similarly, the RanBP2 ZnFs of Nup153 recognize RanGTP/GDP by using a Leu-Val motif in the same position. The RanBP2 ZnF in the putative regulator of cytokine signaling TRABID/ZRANB1 contains several of the RNA-binding residues and a Thr-Tyr motif. Based on such analysis the inventors have reasoned that there exists a family RanBP2 ZnF RNA-Binding that directly bind with high specificity to ssRNA containing a core GGU sequence. Functional studies e.g., as shown in FIG. 23 support this conclusion.

2.8.1 Binding of EKLF Domains to ssRNA

This example demonstrates that EKLF is a functional member of the family of RanBP2-type zinc finger domain proteins.

The inventors produced a construct having three tandem zinc finger domains of the transcription factor EKLF, i.e., EKLF-F123, which binds to CACCC motif in dsDNA (Miler, I. J. and Bieker, J. J., 1993 Mob Cell Biol, 13: 2776-86). The inventors also produced a construct having two tandem zinc finger domains of the transcription factor EKLF, i.e., EKLF-F23. Gel shift analysis by the inventors showed that EKLF-F123, but not EKLF-F23 also bound to ssRNA and that EKLF-F123 has enhanced specificity for polyU ssRNA compared to polyC or an AGGUAA sequence. The fluorescence data demonstrated that the interaction is stoichiometric and indicate that each zinc finger domain is likely to contact three bases. See e.g., FIGS. 32A-32C hereof.

### Example 3

### Mutations of Arginine-82 Alter ssRNA Binding Preference of ZRANB2-F2

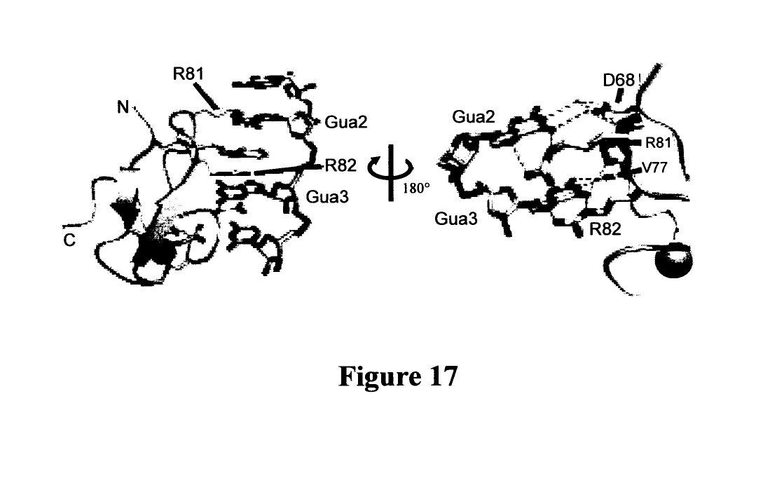

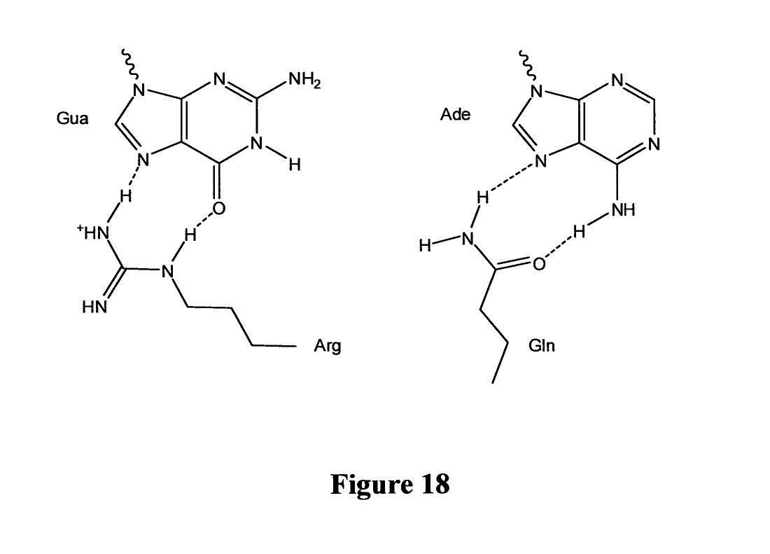

This set of experiments investigates the effect of mutating R82 in the second zinc finger domain (F2) of ZRANB2 on the binding affinity of ZRANB2 to GGU in ssRNA. Arginine recognizes guanine by specific hydrogen bonds, and when ZRANB2-F2 binds to AGGUAA, arginine-81 (R81) and arginine-82 (R82) each form two hydrogen bonds with the guanine bases i.e., with Gua2 and Gua3, respectively (e.g.; FIGS. 17, 18). Similarly, glutamine recognizes adenine through two hydrogen bonds between the glutamine side chains and the adenine base (e.g., FIG. 18).

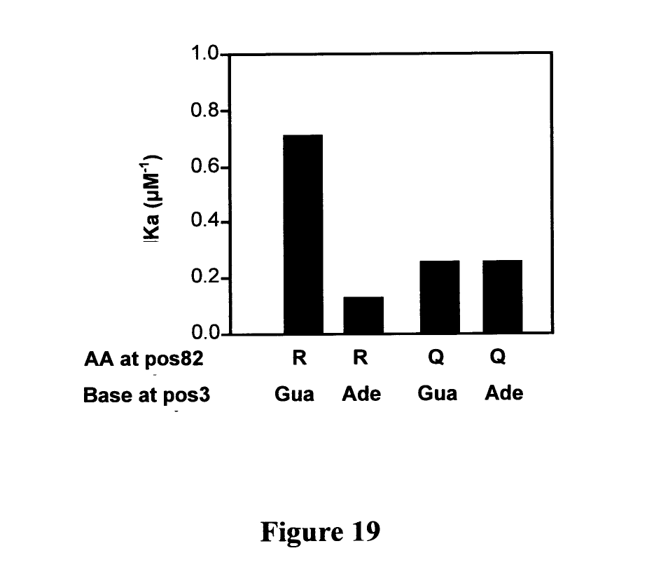

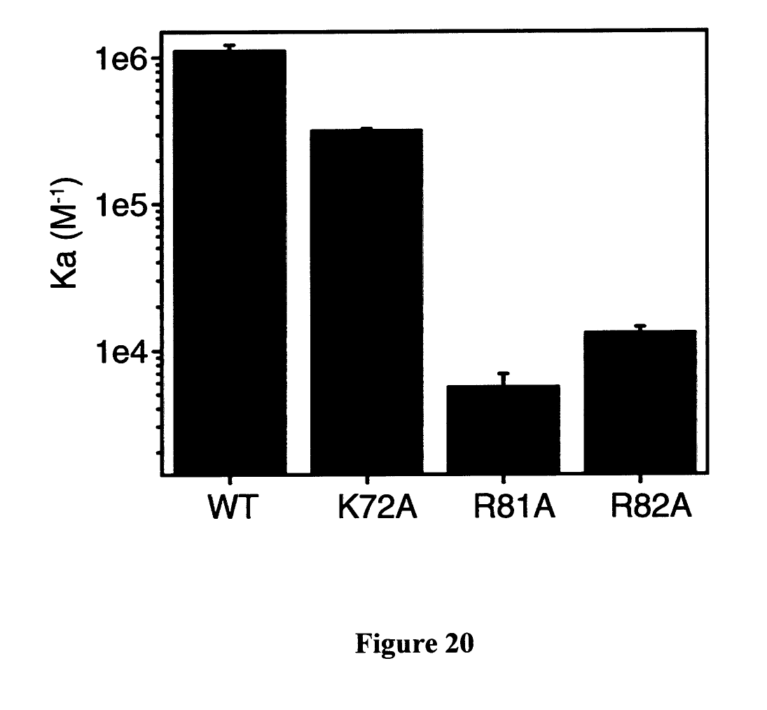

To determine whether changing arginine-82 (R82) would affect the binding preference of ZRANB2-F2 from GGU to GAU, the point mutation R82Q in ZNF 2 of ZRANB2 was made and the ability of both the wild type (W) protein and the R82Q mutant to bind to AGGUAA and AGAUAA was assessed by fluorescence anisotropy binding assays as described previously in Example 1. The results are shown in FIG. 19 and FIG. 20. Data demonstrate that the RNA binding affinity of WT ZRANB2-F2 to guanine base was about 4-fold higher than its binding affinity to adenine base, however the R82Q mutant protein showed no preferential binding affinity between guanine and adenine bases. The results also demonstrate that the point mutation R82Q resulted in about 2 fold increase in the affinity of ZnF2 of ZRANB2 for AGAUAA and about 2 fold decrease in the affinity of ZnF2 of ZRANB2 for AGGUAA.

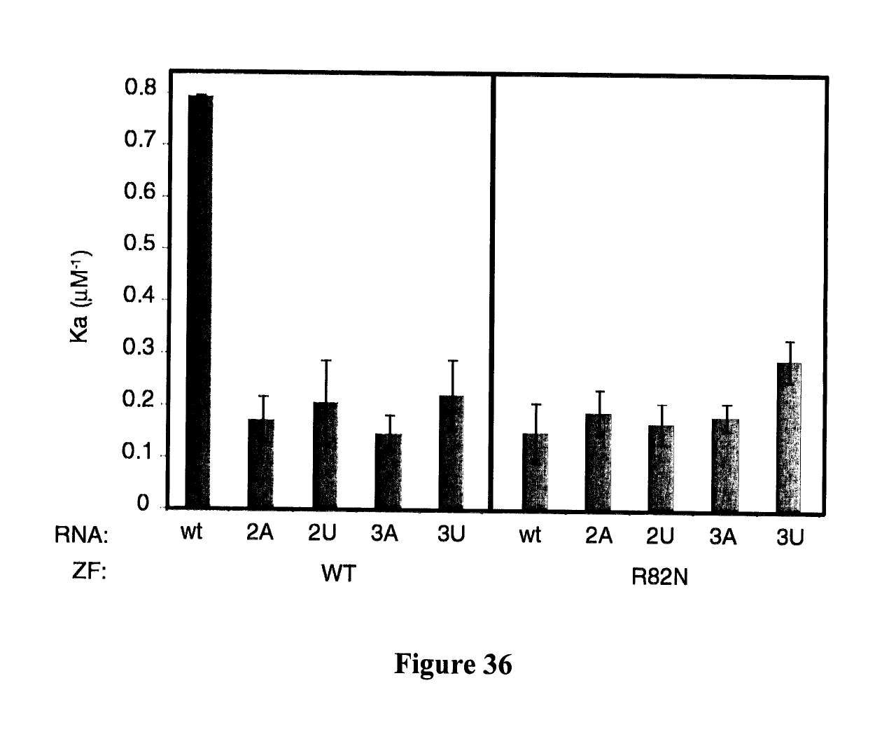

To determine whether changing arginine-82 (R82) would affect the binding preference of ZRANB2-F2 from GGU to GUU, the point mutation R82N in ZNF 2 of ZRANB2 was made and the ability of both the wild type (W) protein and the R82N mutant to bind to AGGUAA and each of a series of mutant sequences, i.e., comprising adenine at position 2 of the wild-type sequence i.e., the sequence AAGUAA, or uridine at position 2 of the wild-type sequence i.e., the sequence AUGUAA, or adenine at position 3 of the wild-type sequence the sequence AGAUAA, or uridine at position 3 of the wild-type sequence i.e., the sequence AGUUAA, was assessed by fluorescence anisotropy binding assays as described in Example 1. The results are shown in FIG. 36. Data demonstrate that the RNA binding affinity of WT ZRANB2-F2 to GGU was about 2.5-fold to 3-fold higher than its binding affinity to GUU, and about 4-fold to 8-fold higher than its binding affinity to GAU. However, the R82N mutant protein showed about 2-fold higher binding affinity to GUU-compared to GGU, indicating a preference of R82N for uridine at position 3. Thus, these data demonstrate that the point mutation R82N resulted in about 2-fold increase in the affinity of ZnF2 of ZRANB2 for AGUUAA and about 2.5-fold to 3-fold decrease in the affinity of ZnF2 of ZRANB2 for AGGUAA.

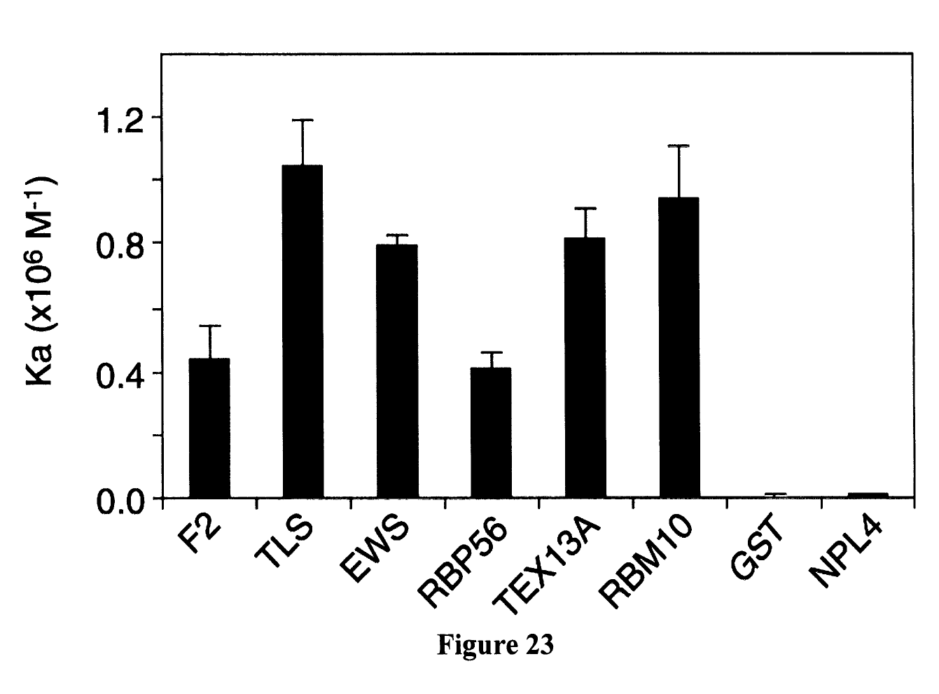

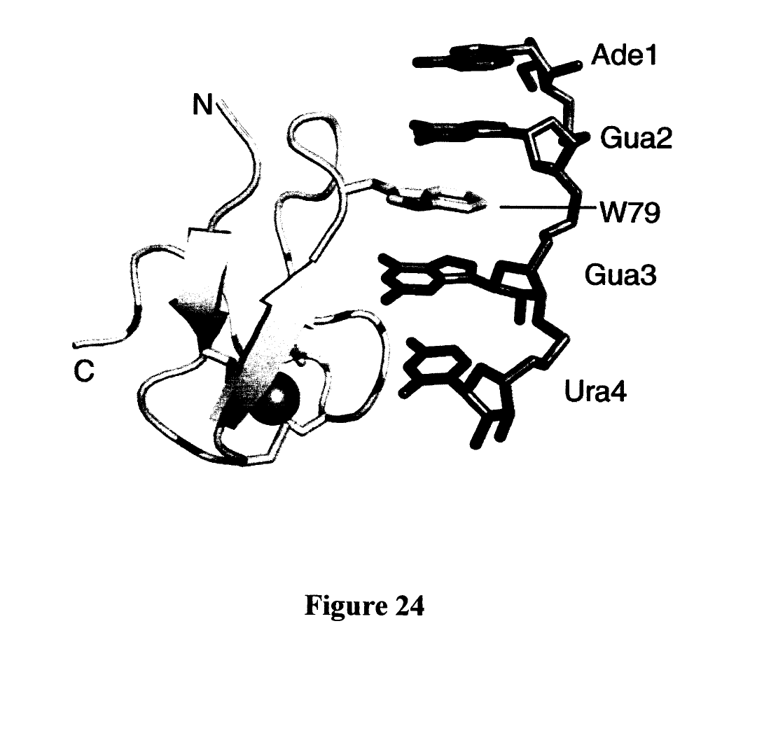

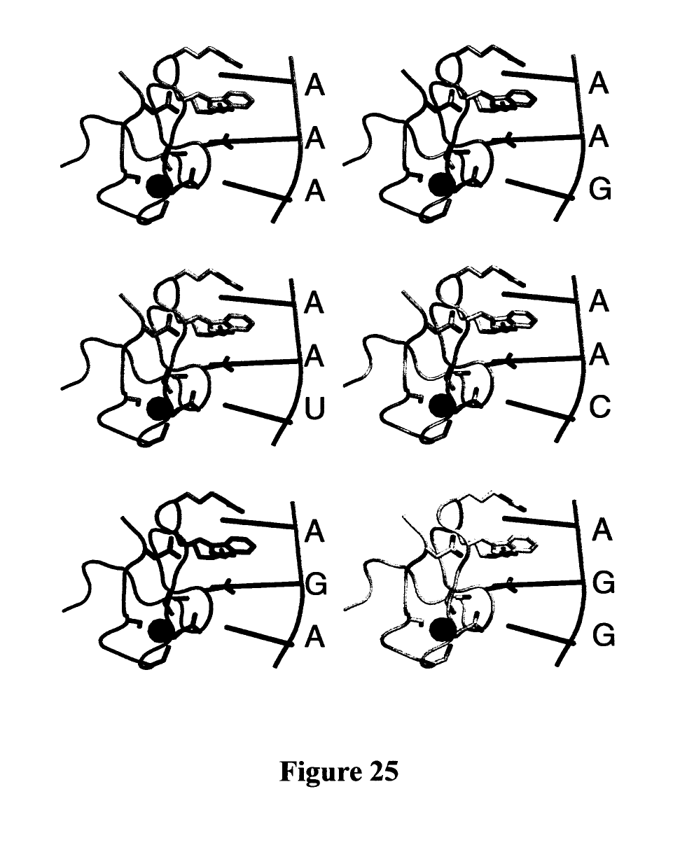

These data demonstrate that mutating specific bases in the core sequence of the ssRNA can create “rationally designed” mutants that may be used to screen for ZRANB2 and/or ZnFs that have varying specificity for different ssRNA triplets. Based on modeling of the interface between the F2 domain of ZRANB2 and the core substrate sequence as shown in FIG. 24, ribbon and stick representations showing putative associations of divergent RanBP2-type zinc finger domains to ssRNAs comprising different tri-ribonucleotide core sequences are provided in FIG. 25.

### Example 4

### Multimerization of RanBP2 Zinc Finger Domains Enhances Binding Affinity

This example demonstrates higher affinities of binding of polypeptides comprising three RanBP2 zinc finger domains compared to polypeptides comprising only two copies of the same RanBP2 zinc finger domains.

To evaluate the possibility of creating ZRANB2 zinc finger motif constructs with larger arrays of zinc finger motifs and with a designed ability to bind extended consensus sequences, a 3 ZnF ZRANB2 construct (F122) was designed and synthesized based on the Δ45-64 F12 2ZnF construct.

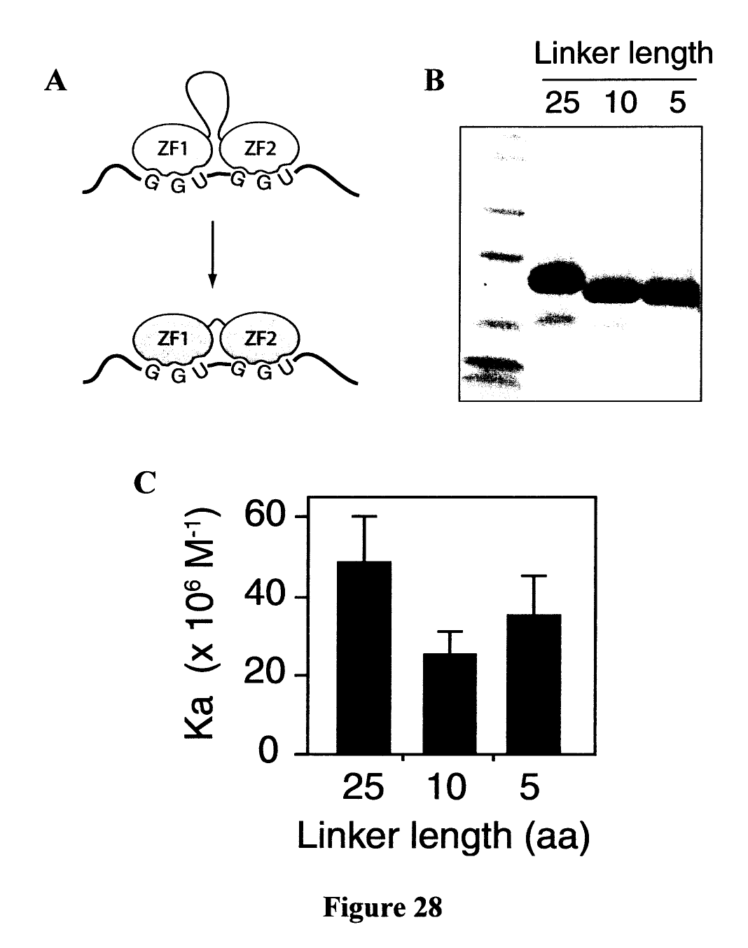

As shown in FIG. 16 and FIG. 28C, ssRNA consisting of the sequence 5′-AAAGGUGGUAAAA-3′ (SEQ ID NO: 27) binds to a trimeric zinc finger polypeptide comprising a single F1 domain and two F2 domains of ZRANB2 in tandem (F122) at higher affinity than the same sequence binds a dimeric zinc finger polypeptide comprising a single. F1 domain and a single F2 domain of ZRANB2 in tandem with linker residues 45-64 deleted (F12 Δ45-64). Using fluorescence anisotropy the ability of the ZRANB2-F122 construct to bind a 5′-AAGGUGGUGGUAA-3′ (SEQ ID NO: 42) ssRNA motif was assayed and found it binds on average with a 3-4 fold higher affinity to this motif compared with F12 (Δ45-64), displaying K_{A} values of ˜84×10⁶ M⁻¹ and ˜25×10⁶ M⁻¹, respectively.

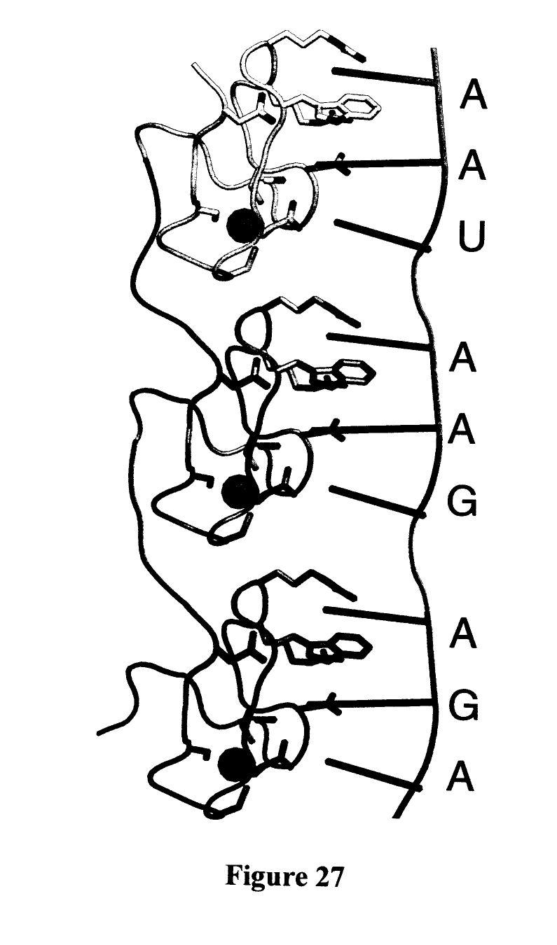

Based on modeling of the interface between the F2 domain of ZRANB2 and the core substrate sequence as shown in FIG. 24, ribbon and stick representations are provided herein as FIG. 27, showing putative associations of a multimeric composition of the invention comprising F1-F2-F2 of ZRANB2 to ssRNA comprising repeats of different tri-ribonucleotide core sequences that bind the domains of the multimeric composition. FIG. 27 demonstrates binding of multimeric compositions of the invention to substrate ssRNAs having divergent core sequences in their RanBP2 zinc finger domains.

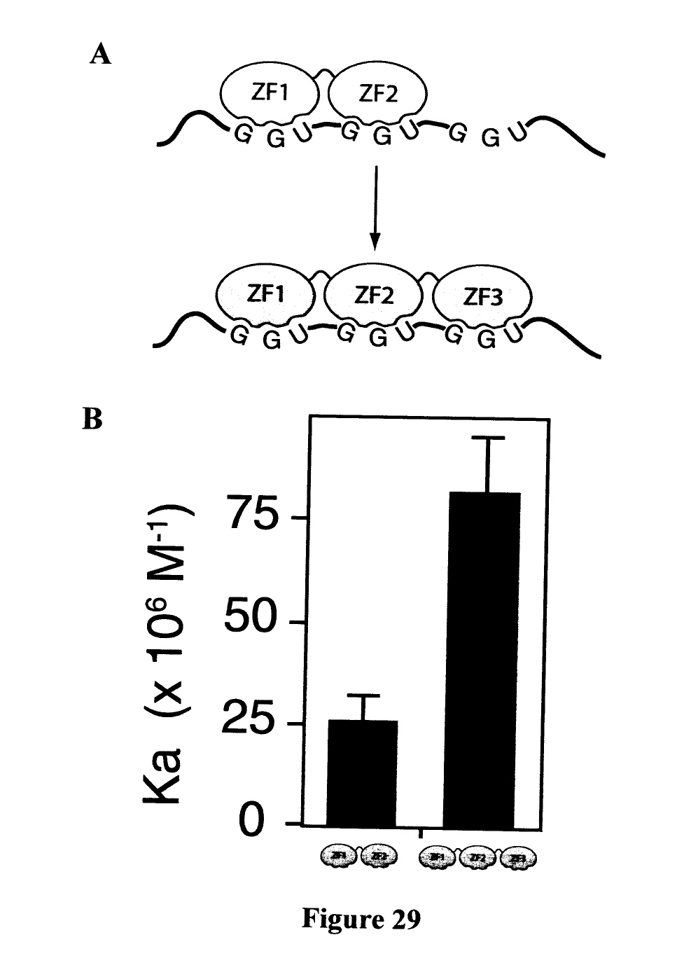

The binding of a multimeric composition of the invention to ssRNA is shown generally in FIG. 29A, wherein: (i) a multimeric composition of the invention comprising two non-contiguous RanBP2-type zinc finger domains ZF1 and ZF2, separated by a linker region to ssRNA comprising three repeats of the GGU tri-ribonucleotide core sequence (above); and (ii) a multimeric composition of the invention comprising three non-contiguous RanBP2-type zinc finger domains ZF1 and ZF2 and ZF3 to ssRNA comprising three repeats of the GGU tri-ribonucleotide core sequence (below) bind to the same ssRNA target occupying specific sites. As shown in FIG. 29B is a graphical representation showing the associations of ssRNA comprising three repeats of the GGU tri-ribonucleotide core sequence to the constructs of FIG. 29A wherein there is enhanced binding affinity of the trimeric zinc finger polypeptide to ssRNA relative to the dimeric form.

In another example, a combinatorial RanBP2-type zinc finger domain comprising one or more zinc finger domains from EKLF is produced having modified specificity relative to naturally-occurring EKLF e.g., using one or more EKLF zinc finger domains covalently linked to one or more other RanBP2-type zinc finger domains.

### Example 5

### Optimal Linker Compositions for Assembling Modular ssRNA-Binding Zinc Finger Proteins

This example examines optimisation of optimal linker length and/or composition for the assembly or construction of modular ssRNA-binding zinc finger proteins comprising different classes of domains which may have different sequence biases.

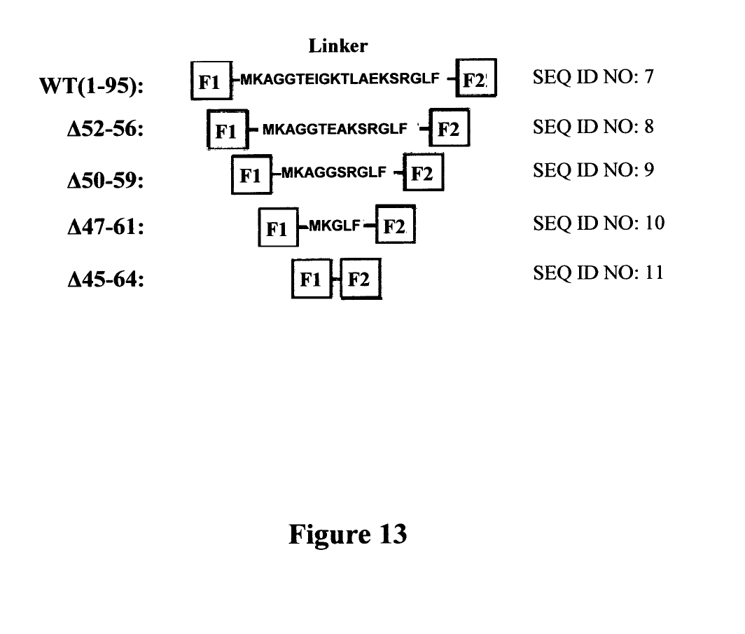

The inventors have employed deletion mutagenesis to thereby identify a minimal inter-finger linker region of ZRANB2 required for specific ssRNA-binding. A series of deletion mutants was produced wherein about 5-20 amino acids were progressively removed from the inter-finger linker region of the ZRANB2 (1-95), thereby producing inter alia the zinc finger deletion constructs shown in FIG. 13. The deletion constructs were then tested for an ability to bind the ssRNA substrate 5′-GGUNXGGU-3′ comprising a repeat of the GGU core sequence using fluorescence anisotropy titration.

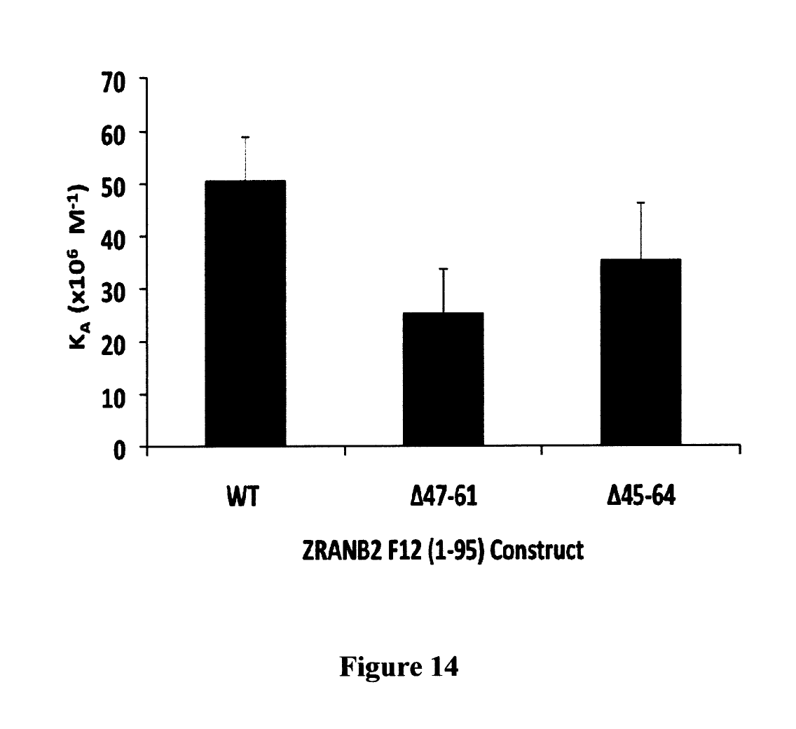

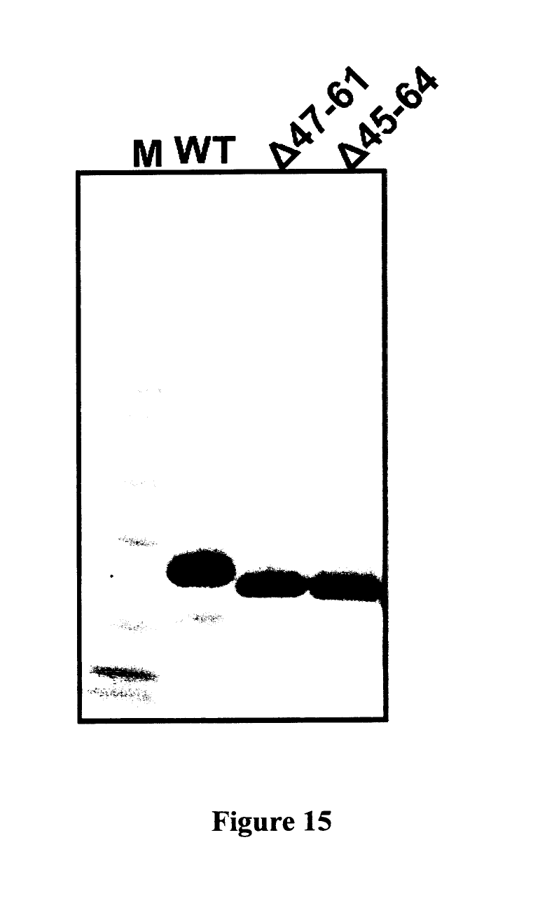

Data presented in FIG. 14 demonstrate that even after removing 15 internal amino acids from the inter-finger linker region, as in construct M7-61, binding affinity for the ssRNA substrate was of a comparable order to the wild-type construct (K_{A} about 30-51×10⁶ M⁻¹). Furthermore, the Δ47-61 construct was stable and resistant to degradation as determined by the absence of lower molecular weight degradation products when resolved by SDS-PAGE e.g., FIG. 15 and FIG. 28B.

Accordingly, the available data suggest that an inter-finger linker comprising a sequence selected from SEQ ID NOs: 22-24 is sufficient for binding between the flanking RanBP2-type zinc finger domains and ssRNA substrate comprising GGU core sequences, wherein:

(i)
SEQ ID NO: 22 consists of the sequence
-MKAGGTEAEKSRGLF;
(ii)
SEQ ID NO: 23 consists of the sequence
MKAGGSRGLF;
and
(iii)
SEQ ID NO: 24 consists of the sequence
MKGLF.

### Example 6

### Combinatorial Libraries and their Use

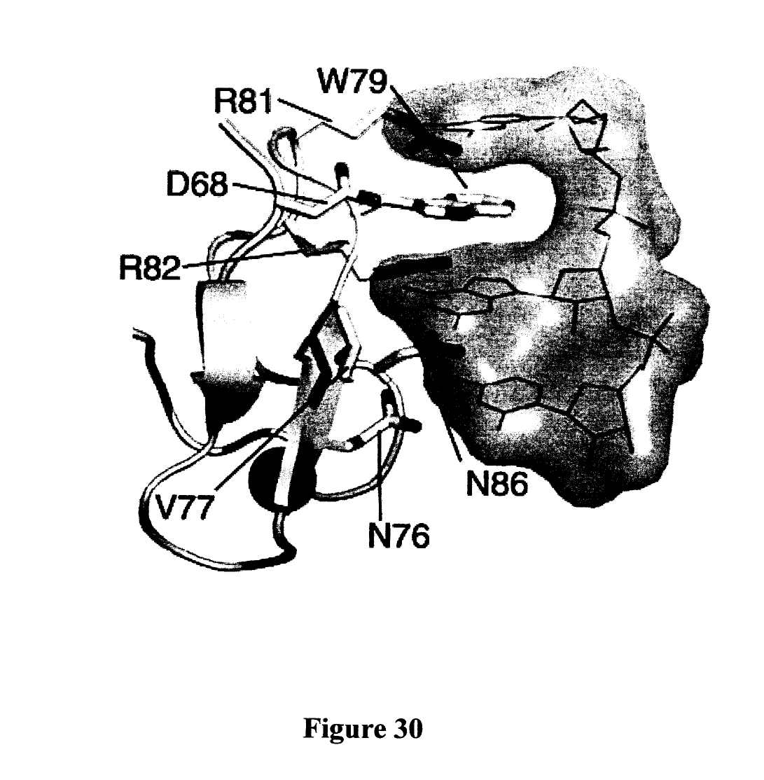

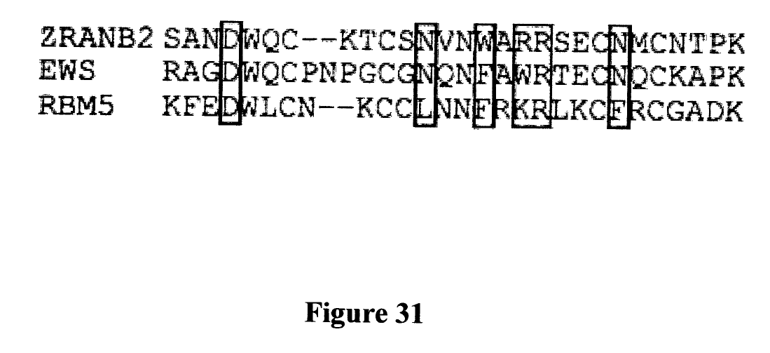

Based on the data provided by way of Example 3 hereof, combinatorial libraries comprising a plurality of RanBP2-type zinc finger domains are produced, e.g., wherein residues lying on the RNA-binding surface of individual clones are randomized, to enhance diversity of binding specificity. In the case of the RanBP2 ZnF, the core sequence from finger 2 of ZRANB2 (i.e., ZRANB2-F2) is used and the seven residues that contact RNA in the X-ray structure are randomized (for example, FIG. 30).

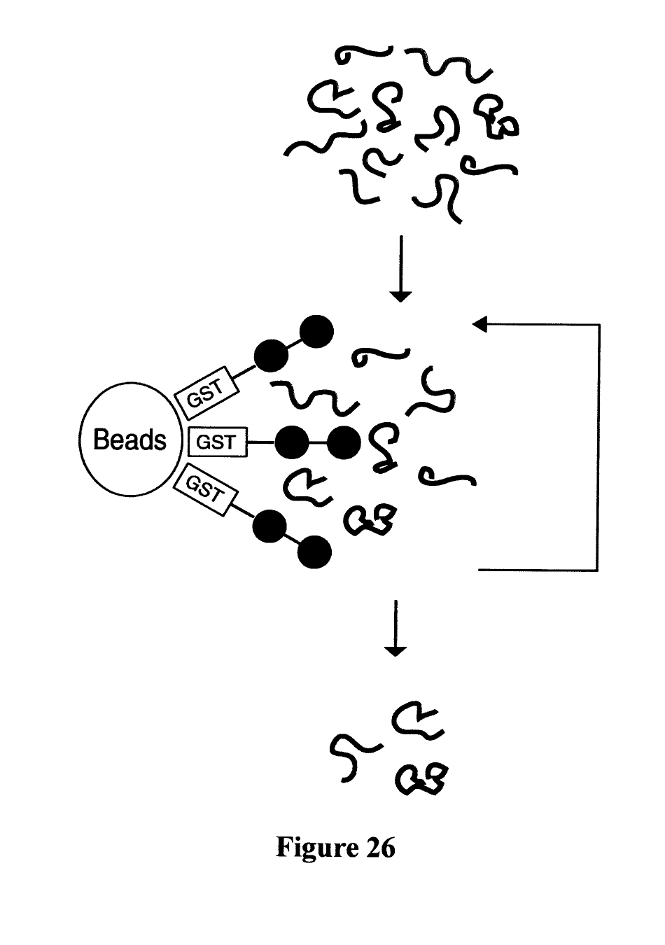

Alternatively, or in addition, by further combining monomeric RanBP2-type zinc finger domains to form higher order molecules such as dimmers and trimers etc, diversity and specificity are further enhanced. Thus, modular ssRNA-binding proteins with tailored specificities e.g., as shown in the display library depicted in FIG. 26 hereof. For example, such combinatorial libraries and individual clone4s thereof are useful as diagnostic reagents to investigate and regulate cellular and/or therapeutic processes e.g., ssRNA localization.

In one example, a RanBP2-type zinc finger domain library is created by gene synthesis using degenerate nucleotides to code for the residues that are to be randomized, wherein the identity of the degenerate nucleotides is selected to provide an NNK codon bias at each randomized position (for example, N=G, C, T, A; K=G or T). This strategy encodes all 20 amino acids using 32 different codons, and has the advantages over NNN codons of (i) eliminating two of the three stop codons and (ii) providing a more uniform distribution of the 20 amino acids. The DNA encoding this degenerate ZnF is flanked on each side by a wild-type ZRANB2-F2 domain connected by linker sequences, and cloned in-frame at the 3′ end of gene VIII from the filamentous Ff phage. The gene VIII protein is present in about 2700 copies on the surface of the phage.

To search for protein sequences capable of binding to a given ssRNA sequence, the protein library is screened against ssRNA with the sequence GGUXXXGGU, where XXX represents the ssRNA sequence of interest. For a given sequence XXX, this screen is then repeated using a classical ZnF in the central position. Novel proteins that display specificity for certain nucleotide triplets are assessed using an array of biophysical approaches to determine the molecular basis for their specificity, for example fluorescence anisotropy and NMR spectroscopy for the analysis of protein: ssRNA acid interactions as detailed in Example 1.

The combinatorial library is based further on the optimum linker length and sequence e.g., as determined in the preceding example or established by sequence randomization and phage display selection to identify and isolate linkers that bind with the highest affinity to a GGU repeat.

In screening such libraries, SELEX may be employed as described herein, wherein multiple rounds of binding and amplification are used to select members of a random ssRNA library that bind most strongly to a zinc finger domain of the combinatorial library. Cloning and sequencing of highly selected oligonucleotides then reveals the consensus sequence to which the protein binds most tightly. Alternatively, a clone of the combinatorial library is incubated with a pool of random oligonucleotides and, following stringent washing, the retained oligonucleotides are sequenced directly using deep sequencing. This latter approach provides quantitative information on binding preferences, which is valuable in assessing the selectivity provided by different amino acids for particular RNA sequences. Because of the parallel nature of the sequencing experiment, the inventors are able to explore the binding preferences of a large number of RanBP2-type zinc finger domains simultaneously.

### Example 7

### Cell Based Assays

These set of experiments examine the ability of RanBP2-type zinc finger domains or analogs or variants thereof to display specific RNA binding activity in cellular assays. For example, any one or more of at least three different cell-based assays is employed.

7.1 mRNA Splicing Assay

A schematic representation showing the basis of a splicing assay is presented in FIG. 33. By way of background, the inclusion or exclusion of an alternatively spliced exon generally relies on the binding of splicing factors to sequences within that exon. Such sequences are known as exonic splicing enhancers (ESEs) and exonic splicing silencers (ESSs), respectively. Splicing factors that contain arginine-serine rich (RS) domains promote exon inclusion by binding to ESE elements and using their RS domains to recruit the spliceosome (FIG. 33A). In contrast, hnRNP proteins bind to ESIs and block exon inclusion through their arginine-glycine (RG) rich domains (FIG. 33B).

Minigene splicing assays are used to test the effectiveness of altering splice site choice. Artificial ‘minigenes’ that contain three exons with intervening introns are constructed synthetically. Splicing enhancer or silencer sequences are incorporated into the central exon, creating minigenes that favour inclusion or exclusion of that exon, respectively. Transfection of such constructs into mammalian cells allows the extent of exon inclusion to be quantified by RT-PCR. RanBP2-type zinc finger domain polypeptide of the present invention are sythesized that recognize a specific sequence within the central exon and are fused to either an RS-rich domain or an RG-rich domain. Co-transfection of a plasmid encoding, for example, the polypeptide and RS-domain construct allows the user to determine inclusion of the central exon in the spliced transcript.

Once it is established that such the splicing factors are viable, the splicing of transcripts having clinical relevance is performed. For example, spinal muscular atrophy is a neurodegenerative disorder caused by deletion or mutation of the SMN1 gene. A near-identical paralog, SMN, differs only by a translationally silent C to T mutation in exon 7 that causes substantial skipping of this exon. This skipping event destabilizes the resultant protein, and only low levels of functional SMN2 are normally observed. Increased expression of SMN2 can reduce the severity of SMA, as is observed when multiple copies of SMN2 are present. It has been demonstrated that the C to T mutation disrupts an exonic splicing enhancer motif. A RanBP2-type zinc finger domain polypeptide of the present invention comprising an RS domain and an RNA-binding domain that can recognize a sequence within exon 7 is tested for an ability to increase SMN2 levels in HEK293 cells (in which SMN2 splicing has previously been monitored).

7.2 Translational Assay In one example of this assay, translation of a reporter mRNA is repressed by fusion a RanBP2-type zinc finger domain polypeptide of the present invention to human Argonaut 2 (Ago2). The fusion protein is made to specifically bind a single-stranded miRNA target site, thereby targeting Ago2 in an miRNA independent manner. Co-transfection of plasmids encoding the luciferase and the RBP-Ago2 fusion leads to an attenuation of luciferase translation compared to controls.

Alternatively or in addition, translation of specific mRNAs is activated by fusing a RanBP2-type zinc finger domain polypeptide to the central ribosome recruitment domain of eIF4G. This approach is clinically beneficial in disorders such as FXTAS, where levels of FMRP mRNA are normal but protein levels are significantly reduced. This principle is tested using bi-cistronic reporter mRNA where expression of the two reporters is measured in transfected cells by luminometry.

A protein is constructed comprising eIF4G fused to a RanBP2-type zinc finger domain polypeptide that then targets the inter-cistronic region of the reporter and measures expression of two reporters in the presence of varying amounts of a plasmid encoding the fusion.

After demonstrating the activation of translation using the fusions supra, the fusion proteins are employed to alter the translation of the dystrophin gene, mutations of which give rise to Duchenne muscular dystrophy. Cells are transfected with dystrophin minigene constructs containing mutations in the first exon and translation factors targeted to downstream sequences in the gene are used to improve expression of this gene.

### 7.3 RNA Labeling for Cellular Localization Studies

The ability to specifically tag RNAs with fluorescent labels provides a powerful means of imaging mRNAs as they move within living cells. However, current approaches for imaging mRNA movement are problematic. Most of the widely used approaches involve genetic modification of the target RNA, for example through the addition of RNA elements to which a specific protein binding partner is available, to permit specific labelling.

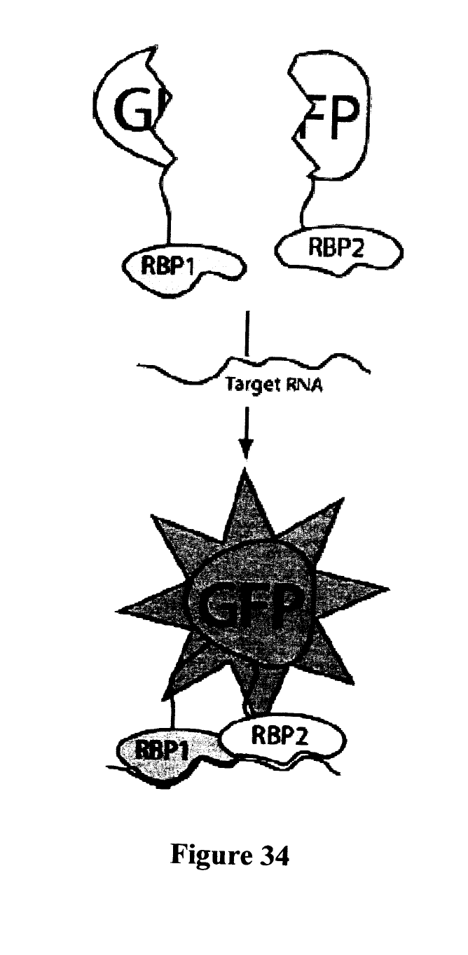

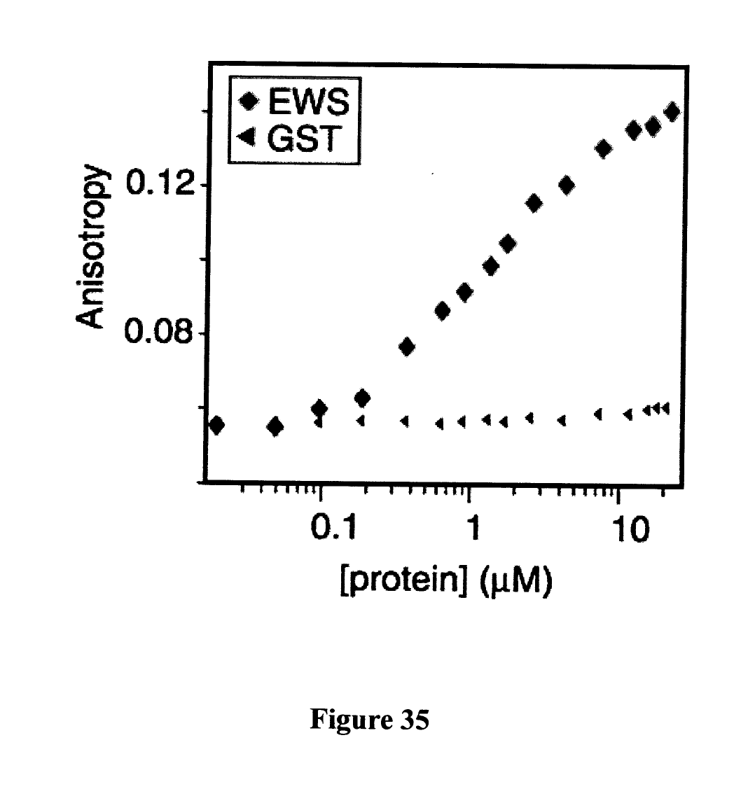

In one example, a schematic representation of a bimolecular fluorescence complementation (BiFC) is presented in FIG. 34. The bimolecular fluorescence complementation (BiFC) approach is used to create a high signal-to-noise system for tagging and monitoring endogenous RNA species in vivo in real time. The approach is based on the association of two non-fluorescent fragments of a fluorescent protein when they are brought into proximity by an interaction involving proteins that are fused to each fragment. Two RanBP2-type zinc finger domain polypeptides are designed according to any example hereof to bind to nearby sequences in a target RNA and each of these RanBP2-type zinc finger domain polypeptides is fused to half of a fluorescent protein. Fluorescence is observed only when both RanBP2-type zinc finger domain polypeptides bind to their target sequences, providing very high specificity.

### 7.4 Other Cell Based Assays

Zinc finger ribonucleases are used for the sequence specific cleavage of specific ssRNA species, such as retroviral RNA within infected cells. RanBP2-type zinc finger domain polypeptides according to any example hereof that target internal ribosome entry sites (IRES) are used to block viral translation without significantly interfering with translation of host proteins e.g., in the treatment of retroviral infection e.g., hepatitis C virus, rhinovirus, or HIV-1 infection.

We claim:

1. A composition comprising an amount of at least one RanBP2-type zinc finger domain e.g., as defined herein above, or a variant or analog thereof that binds to single-stranded RNA (ssRNA), wherein said ssRNA comprises at least one occurrence of a sequence that binds to a RanBP2-type zinc finger domain or variant or analog.

2. The composition according to claim 1 wherein said composition is a peptide or polypeptide comprising at least one RanBP2-type zinc finger domain, variant or analog.

3. The composition of claim 1, wherein at least one RanBP2-type zinc finger domain comprises Structural Formula II X₂₋₃-Za-X₀₋₁-W-X-C-X₂₋₄-C-X-Zb-X₂-Zc-X-Zd-Ze-X₂-C-Zf-X-C or a variant or analog thereof, wherein each of X, Za, Zb, Zc, Zd, Ze and Zf is an amino acid, and wherein a side chain of any one or more of Za to Zf is functional to contact at least one residue of single-stranded RNA such that W intercalates between two residues of a sequence-specific binding site in single-stranded RNA (ssRNA).

4. The composition of claim 3, wherein Za is selected from amino acid residues of the group consisting of D, T, S, N and A.

5. The composition of claim 3, wherein Zb is selected from amino acid residues of the group consisting of N, A, L, V, E, K, Y and F.

6. The composition of claim 3, wherein Zc is selected from amino acid residues of the group consisting of F, W, K, A, P, S, W Q.

7. The composition of claim 3, wherein Zd is selected from amino acid residues of the group consisting of R, W, K, E, T, L, S and G.

8. The composition of claim 3, wherein Zd is selected from the group consisting of R, K, E, T, L, S and G.

9. The composition of claim 3, wherein Ze is selected from amino acid residues of the group consisting of R, A, P, K, T, Q and N.

10. The composition of claim 3, wherein Zf is selected from N, F, V, T, and E.

11. The composition of claim 1 comprising a plurality of the RanBP2-type zinc finger domains and/or variants and/or analogs.

12. The composition of claim 11, wherein two or more RanBP2-type zinc finger domains and/or variants and/or analogs are linked contiguously in a polypeptide.

13. The composition of claim 11, wherein two or more RanBP2-type zinc finger domains and/or variants and/or analogs are spaced apart by an inter-finger linker molecule.

13. The composition of claim 11, wherein two or more RanBP2-type zinc finger domains and/or variants and/or analogs are identical.

13. The composition of claim 11, wherein two or more RanBP2-type zinc finger domains and/or variants and/or analogs are different.

14. The composition of claim 13, wherein two or more inter-finger linker molecules are present and they are the same.

15. The composition of claim 13, wherein two or more inter-finger linker molecules are present and they are different.

16. The composition of claim 13 wherein an inter-finger linker molecule comprises a sequence having at least about 80% identity to a sequence selected from SEQ ID NOs: 22-24.

17. The composition of claim 1, wherein the RanBP2-type zinc finger domain or a variant or analog thereof has specificity for a ssRNA substrate comprising at least one occurrence of the sequence GGU or GAU or GUU or AGU or AAU that binds to a RanBP2-type zinc finger domain or a variant or analog thereof.

18. The composition of claim 1, wherein the RanBP2-type zinc finger domain or a variant or analog thereof has specificity for a ssRNA substrate comprising at least one occurrence of a polyuridine sequence.

19. A diagnostic reagent comprising the composition of claim 1 wherein one or more RanBP2-type zinc finger domains and/or variants and/or analogs is fused to a detectable reporter molecule.

20. An isolated polypeptide comprising at least one RanBP2-type zinc finger domain or a variant or analog thereof that binds to single-stranded RNA (ssRNA), wherein the polypeptide is other than a naturally-occurring ZnF protein.

21. The isolated polypeptide of claim 20 comprising a structure selected individually or collectively from the group consisting of
(i) Structural Formula II:
X₂₋₃-Za-X₀₋₁-W-X-C-X₂₋₄-C-X-Zb-X₂-Zc-X-Zd-Ze-X₂-C-Zf-X-C,
wherein each of X, Za, Zb, Zc, Zd, Ze and Zf is an amino acid, and wherein a side chain of any one or more of Za to Zf is functional to contact at least one residue of single-stranded RNA such that W intercalates between two residues of a sequence-specific binding site in single-stranded RNA (ssRNA);
(ii) a functional fragment of (i);
(iii) a peptidyl fusion comprising a plurality of structures of said Structural Formula II and/or said functional fragments, optionally wherein at least two of said plurality are separated by a linker molecule;
(iv) any one of (i) or (ii) or (iii) additionally comprising a protein transduction domain or a retroinverted analog thereof and/or a serum protein-binding moiety, optionally wherein (i) or (ii) or (iii) is separated from the protein transduction domain and/or serum protein-binding moiety by a spacer or said protein transduction domain and/or serum protein-binding moiety are separated by one or more spacers; and
(v) an analog of any one of (i) to (iv) comprising one or more non-naturally-occurring amino acids or non-naturally-occurring amino acid analogs, or an isostere of any one of (i) to (iv), or a retro-peptide analog of any one of (i) to (iv), or a retro-inverted peptide analog of any one of (i) to (iv).

22. The composition of claim 1 comprising a plurality of the isolated RanBP2-type zinc finger domains and/or variants and/or analogs thereof or a plurality of isolated polypeptides comprising said RanBP2-type zinc finger domains or variants or analogs.

23. The composition of claim 22, wherein a plurality of isolated RanBP2-type zinc finger domains and/or variants and/or analogs thereof or a plurality of isolated polypeptides comprising same is arrayed separately on a solid substrate.

24. The composition of claim 23, wherein the solid substrate is a microchip, a bead, a particle or a nanoparticle.

25. The composition of claim 22, comprising an admixture of a plurality of isolated RanBP2-type zinc finger domains and/or variants and/or analogs thereof or a plurality of isolated polypeptides comprising said RanBP2-type zinc finger domains or variants or analogs.

26. An isolated polynucleotide other than a naturally-occurring ZnF protein-encoding gene, wherein the polynucleotide encodes at least one RanBP2-type zinc finger domain or a variant or analog thereof that binds to single-stranded RNA (ssRNA) or an isolated polypeptide comprising said RanBP2-type zinc finger domain(s) or variant(s) or analog(s).

27. An expression vector comprising the polynucleotide according to claim 26 capable of expressing a one or more isolated RanBP2-type zinc finger domains and/or variants and/or analogs thereof or an isolated polypeptide comprising same.

28. The expression vector of claim 27 comprising a phagemid capable of expressing a one or more isolated RanBP2-type zinc finger domains and/or variants and/or analogs thereof or an isolated polypeptide comprising said RanBP2-type zinc finger domain(s) or variant(s) or analog(s).

29. The expression vector of claim 27 for use in human or other animal cells or in plant cells.

30. A formulation comprising the composition of claim 1 a pharmaceutically acceptable carrier and/or excipient.

31. The formulation according to claim 30 wherein the carrier or excipient comprises one or more protease inhibitors and/or RNase enzymes.

32. The formulation according to claim 30 wherein the carrier or excipient comprises one or more protease inhibitors and/or RNase inhibitors.

33. A method for producing a formulation according to claim 30 said method comprising mixing or otherwise combining one or more isolated RanBP2-type zinc finger domains and/or variants and/or analogs thereof or an isolated polypeptide comprising same in an amount sufficient to modify ssRNA expression with a suitable carrier or excipient.

34. Use of the composition of claim 1 in medicine.

35. Use of the formulation of claim 30 in medicine.

36. Use of the composition of claim 1 in a method of treatment of the human or animal body by prophylaxis or therapy.

36. Use of the composition of claim 1 in a method of drug screening, drug development or clinical trial.

37. Use of the composition of claim 1 in a method of modulating expression of mRNA splice variants associated with a disease state or to modify splicing of one or more mRNA transcripts.

38. Use of the composition of claim 1 in a method of prophylaxis and/or therapy of one or more adverse effects or consequences of ssRNA expression.

39. Use of the composition of claim 1 in the preparation of a medicament for modulating gene expression associated with ssRNA level in a cell.

40. Use of the composition of claim 1 in a method to regulate or drive translation of mRNA.

41. Use of the diagnostic reagent of claim 19 in a method to determine ssRNA localization in a cell.

42. A method of preventing or treating one or more adverse consequences of ssRNA expression in a subject or in an isolated cell, said method comprising administering an amount of a composition of claim 1 for a time and under conditions sufficient to bind to ssRNA and thereby modulate gene expression.


SEQUENCE LISTING (42 sequences)

SEQ ID NO: 1 - protein, 26 aa, Artificial Sequence
  RanBP2-type zinc finger domain structure
SDGDWICPDKKCGNVNFARRTSCNRC

SEQ ID NO: 2 - protein, 24 aa, Artificial Sequence
  RanBP2-type zinc finger domain structure
SANDWQCKTCSNVNWARRSECNMC

SEQ ID NO: 3 - protein, 26 aa, Artificial Sequence
  RanBP2-type zinc finger domain structure
RAGDWKCPNPTCENMNFSWRNECNQC

SEQ ID NO: 4 - protein, 26 aa, Artificial Sequence
  RanBP2-type zinc finger domain structure
RAGDWQCPNPGCGNQNFAWRTECNQC

SEQ ID NO: 5 - protein, 26 aa, Artificial Sequence
  RanBP2-type zinc finger domain structure
KSGDWVCPNPSCGNMNFARRNSCNQC

SEQ ID NO: 6 - protein, 24 aa, Artificial Sequence
  RanBP2-type zinc finger domain structure
RPGDWDCPWCNAVNFSRRDTCFDC

SEQ ID NO: 7 - protein, 24 aa, Artificial Sequence
  RanBP2-type zinc finger domain structure
KFEDWLCNKCCLNNFRKRLKCFRC

SEQ ID NO: 8 - protein, 24 aa, Artificial Sequence
  RanBP2-type zinc finger domain structure
INEDWLCNKCGVQNFKRREKCFKC

SEQ ID NO: 9 - protein, 24 aa, Artificial Sequence
  RanBP2-type zinc finger domain structure
VIGTWDCDTCLVQNKPEAIKCVAC

SEQ ID NO: 10 - protein, 24 aa, Artificial Sequence
  RanBP2-type zinc finger domain structure
EGSWWHCNSCSLKNASTAKKCVSC

SEQ ID NO: 11 - protein, 24 aa, Artificial Sequence
  RanBP2-type zinc finger domain structure
LADYWKCTSCNEMNPPLPSHCNRC

SEQ ID NO: 12 - protein, 21 aa, Artificial Sequence
  RanBP2-type zinc finger domain structure
SEDEWQCTECKKFNSPSKRYC

SEQ ID NO: 13 - protein, 24 aa, Artificial Sequence
  RanBP2-type zinc finger domain structure
NANKWSCHMCTYLNWPRAIRCTQC

SEQ ID NO: 14 - protein, 24 aa, Artificial Sequence
  RanBP2-type zinc finger domain structure
TAAMWACQHCTFMNQPGTGHCEMC

SEQ ID NO: 15 - protein, 25 aa, Artificial Sequence
  RanBP2-type zinc finger domain structure
FSANDWQCKTCSNVNWARRSECNMC

SEQ ID NO: 16 - protein, 25 aa, Artificial Sequence
  RanBP2-type zinc finger domain structure
FSANDWQCKTCGNVNWARRSECNMC

SEQ ID NO: 17 - protein, 25 aa, Artificial Sequence
  RanBP2-type zinc finger domain structure
FSAEDWQCSKCANVNWARRQTCNMC

SEQ ID NO: 18 - protein, 25 aa, Artificial Sequence
  RanBP2-type zinc finger domain structure
FAAEDWVCSKCGNVNWARRRTCNVC

SEQ ID NO: 19 - protein, 25 aa, Artificial Sequence
  RanBP2-type zinc finger domain structure
FAAEDWICSKCGNVNWARRKTCNVC

SEQ ID NO: 20 - protein, 25 aa, Artificial Sequence
  RanBP2-type zinc finger domain structure
FGPNDWPCPMCGNINWAKRMKCNIC

SEQ ID NO: 21 - protein, 25 aa, Artificial Sequence
  RanBP2-type zinc finger domain structure
FRAGDWKCSTCTYHNFAKNVVCLRC

SEQ ID NO: 22 - protein, 15 aa, Artificial Sequence
  Spacer sequence
MKAGGTEAEKSRGLF

SEQ ID NO: 23 - protein, 10 aa, Artificial Sequence
  Spacer sequence
MKAGGSRGLF

SEQ ID NO: 24 - protein, 5 aa, Artificial Sequence
  Spacer sequence
MKGLF

SEQ ID NO: 25 - RNA, 10 nt, Artificial Sequence
  ssRNA substrate
ccagguaaag

SEQ ID NO: 26 - RNA, 12 nt, Artificial Sequence
  ssRNA substrate
agguaaagguaa

SEQ ID NO: 27 - RNA, 13 nt, Artificial Sequence
  ssRNA substrate
aaaggugguaaaa

SEQ ID NO: 28 - RNA, 17 nt, Artificial Sequence
  ssRNA substrate
gcaaccagguaaagucu

SEQ ID NO: 29 - RNA, 21 nt, Artificial Sequence
  ssRNA substrate
cucugaaucuagguaagaaag

SEQ ID NO: 30 - RNA, 16 nt, Artificial Sequence
  ssRNA substrate
gcgagcgguacguaga

SEQ ID NO: 31 - RNA, 17 nt, Artificial Sequence
  ssRNA substrate
aggaauaagugaagcug

SEQ ID NO: 32 - RNA, 21 nt, Artificial Sequence
  ssRNA substrate
cucugaaucuagguaagaaag

SEQ ID NO: 33 - RNA, 11 nt, Artificial Sequence
  ssRNA substrate
agguaagguaa

SEQ ID NO: 34 - RNA, 13 nt, Artificial Sequence
  ssRNA substrate
agguaanagguaa

SEQ ID NO: 35 - RNA, 14 nt, Artificial Sequence
  ssRNA substrate
agguaannagguaa

SEQ ID NO: 36 - RNA, 15 nt, Artificial Sequence
  ssRNA substrate
agguaannnagguaa

SEQ ID NO: 37 - RNA, 16 nt, Artificial Sequence
  ssRNA substrate
agguaannnnagguaa

SEQ ID NO: 38 - RNA, 17 nt, Artificial Sequence
  ssRNA substrate
agguaannnnnagguaa

SEQ ID NO: 39 - RNA, 18 nt, Artificial Sequence
  ssRNA substrate
agguaannnnnnagguaa

SEQ ID NO: 40 - RNA, 19 nt, Artificial Sequence
  ssRNA substrate
agguaannnnnnnagguaa

SEQ ID NO: 41 - RNA, 20 nt, Artificial Sequence
  ssRNA substrate
agguaannnnnnnnagguaa

SEQ ID NO: 42 - RNA, 13 nt, Artificial Sequence
  ssRNA substrate
aagguggugguaa